(19) United States
(12) Patent Application Publication
(10) Pub. No.: US 2022/0002753 A1
(43) Pub. Date: Jan. 6, 2022

(54) SYSTEMS AND METHODS FOR POLYNUCLEOTIDE SPATIAL ORGANIZATION

(71) Applicant: The Board of Trustees of the Leland Stanford Junior University, Stanford, CA (US)

(72) Inventors: Lei S. Qi, Stanford, CA (US); Haifeng Wang, Stanford, CA (US)

(73) Assignee: The Board of Trustees of the Leland Stanford Junior University, Stanford, CA (US)

(21) Appl. No.: 17/180,535

(22) Filed: Feb. 19, 2021

Related U.S. Application Data
(63) Continuation of application No. PCT/US2019/047867, filed on Aug. 23, 2019.
(60) Provisional application No. 62/722,684, filed on Aug. 24, 2018.
(60) Provisional application No. 62/744,504, filed on Oct. 11, 2018.

(51) Int. Cl. C12N 15/90; C12N 15/85; C12N 15/11; C12N 9/22

(52) U.S. Cl. CPC C12N 15/90; C12N 15/85; C12N 2320/50; C12N 9/22; C12N 2310/20; C12N 15/111

(57) ABSTRACT

Provided herein are systems and methods for the controlling the spatial positioning of a target polynucleotide in a compartment of a cell.

### CROSS-REFERENCE TO RELATED APPLICATIONS

This application is a continuation of International Application No. PCT/US2019/047867, filed Aug. 23, 2019, which claims priority to U.S. Provisional Application No. 62/722,684, filed Aug. 24, 2018 and U.S. Provisional Application No. 62/744,504, filed Oct. 11, 2018, the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No. EB021240 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Feb. 19, 2021, is named 079445-002010US-1238448 SL.txt and is 10,357 bytes in size.

### BACKGROUND

The 3-dimensional (3D) spatial organization of polynucleotides within living cells plays an important role in such processes as regulating and maintaining gene expression, genome stability, and cellular function. For example, genomic sequences that associate with nuclear lamina or the nuclear periphery often exhibit low transcriptional activity, while those that localize to the nuclear interior often exhibit relatively higher activity. Furthermore, the eukaryotic cell nucleus contains many membraneless nuclear bodies, such as Cajal bodies, PML bodies, nucleolus and speckles, that are functionally important in a variety of biological processes. A central goal in genomics and cell biology has been to understand the relationship between genome structure, its organization within various nuclear compartments, and gene expression, but this goal has been constrained by currently available methods.

A correlation between genome organization and cell fate determination has been suggested by numerous studies using microscopy-based imaging (e.g., FISH) and chromosome conformation capture (3C) techniques. For example, during lymphocyte development, the IgH and Igκ loci that are positioned at the nuclear periphery in progenitor cells often relocate to nuclear interior in pro-B cells, a process that is synchronous with the activation and rearrangement of immunoglobulin loci. Similarly, the genomic locus of the proneural transcription factor Ascl1 is located in the nuclear periphery in undifferentiated embryonic stem cells, but relocates to the nuclear interior during neuronal differentiation. Moreover, 3C-based studies have revealed changes in high-resolution chromatin interactions (e.g., topologically associated domains) during development and disease processes. Altogether, these are powerful methods for mapping genome organization and measuring physical interactions of chromatin elements, but they often cannot provide causal links between genome positioning and function and they are unable to measure dynamic changes in living cells.

Nuclear compartments have been observed to play an important role in genome organization and function. Nuclear bodies are proposed to assemble through liquid-liquid phase separation, which is driven by multivalent interactions between proteins and RNAs. De novo nuclear body formation can be nucleated by immobilization of protein or RNA components on chromatin. Among nuclear bodies, Cajal bodies (CBs) are essential for vertebrate embryogenesis, and are abundant in tumor cells and neurons. CBs are marked by a scaffold protein component, Coilin, and play an important role in small nuclear RNA (snRNA) biogenesis, ribonucleoprotein (RNP) assembly, and telomerase biogenesis. The promyelocytic leukemia (PML) nuclear bodies, marked by a tumor suppressor protein, PML, are abundant nucleus dot structures that associate with disease processes including tumor and viral infection. However, how the colocalization of nuclear bodies and chromatin causally affects gene expression and cellular function remains mostly elusive.

To understand such causal relationships, sequence-specific DNA-protein interactions have been exploited to mediate targeted genomic reorganization. This technique utilizes an array of LacO repeats inserted into a genomic locus, which facilitates tethering of the adjacent genomic sequence to the nuclear periphery when combined with Lad fused to a nuclear membrane protein. Using this technique, several studies have reported that repositioning a gene to the nuclear periphery leads to gene repression. However, this technique is not suitable for programmable genome targeting, and is tedious and difficult to implement. For example, creating a stable LacO repeat-containing cell line is a prerequisite for this technique, which already involves many steps such as the random insertion of a large LacO repeat array into the genome, screening for cells containing a single insertion locus, generating stable cell lines, and characterization of the genomic insertion site by FISH. New tools are needed to manipulate the spatial and temporal organization of the genome in a programmable, precise, and targeted manner.

Prokaryotic Class II CRISPR-Cas (Clustered regularly interspaced short palindromic repeats-CRISPR associated) systems have been repurposed as a toolbox (e.g. Cas9 and Cpf1) for gene editing, gene regulation, epigenome editing, chromatin looping, and live-cell genome imaging. Nuclease-deactivated Cas (dCas) proteins coupled with transcriptional effectors or epigenetic modifying domains allow regulation of expression of genes adjacent to the single guide RNA (sgRNA) target site. It remains unknown whether the CRISPR-Cas system can be used to mediate genome organization and reposition the location of chromatin DNA relative to various nuclear compartments within mammalian nuclei.

In view of the foregoing, there exists a need for alternative systems and methods to carry out the spatial organization of target polynucleotides. The present disclosure addresses this and other needs.

### BRIEF SUMMARY

In general, provided herein are systems and methods for programmable polynucleotide re-organization. The systems and methods can couple an actuator moiety with cellular compartment-specific proteins via an inducible system such as a chemically inducible system, and can allow efficient, inducible, and dynamic repositioning of polynucleotides, e.g., genomic loci, to particular cellular positions, e.g., the nuclear periphery, Cajal bodies, and PML nuclear bodies (FIG. 1). The systems and methods can expand existing polynucleotide editing and regulation tools, offering an improved technology to manipulate the 3D organization of polynucleotides relative to cellular compartments, and to study the relationship between macro-scale spatial polynucleotide organization and cellular function.

In one aspect, a system is provided for controlling the spatial positioning of a target polynucleotide in a compartment of a cell. The system comprises a compartment-specific protein linked (e.g., fused) to a first dimerization domain. The system further comprises an actuator moiety that targets the target polynucleotide, wherein the actuator moiety is linked (e.g., fused) to a second dimerization domain that is capable of assembling into a dimer with the first dimerization domain. In some embodiments, the cell is a eukaryotic cell.

In some embodiments, the target polynucleotide comprises genomic DNA. In some embodiments, the target polynucleotide comprises RNA. In some embodiments, the actuator moiety comprises a Cas protein, and the system further comprises a guide RNA that complexes with the actuator moiety and hybridizes to the target polynucleotide (e.g., genomic DNA). In some embodiments, the actuator moiety comprises an RNA-binding protein, and the system further comprises a guide RNA that complexes with the actuator moiety and hybridizes to the target polynucleotide (e.g., RNA). In certain instances, the system further comprises a Cas protein that complexes with the guide RNA. In some embodiments, the RNA-binding protein is ADAR1 or ADAR2 and the guide RNA comprises an ADAR-recruiting RNA (arRNA). In some embodiments, the Cas protein substantially lacks DNA cleavage activity. In some embodiments, the Cas protein is a Cas9 protein, a Cas12 protein, a Cas13 protein, a CasX protein, or a CasY protein. In some embodiments, the Cas12 protein is selected from the group consisting of Cas12a, Cas12b, Cas12c, Cas12d, and Cas12e. In some embodiments, the Cas13 protein is selected from the group consisting of Cas13a, Cas13b, Cas13c, and Cas13d. In certain instances, the Cas13d protein is CasRx. In some embodiments, the actuator moiety comprises a binding protein that hybridizes to the target polynucleotide, wherein the binding protein is a zinc finger nuclease or a TALE nuclease. In some embodiments, the actuator moiety comprises an Argonaute protein complexed with a guide polynucleotide, wherein the guide polynucleotide is a guide RNA or a guide DNA, and wherein the guide polynucleotide hybridizes to the target polynucleotide.

In some embodiments, the compartment-specific protein is selected from the group consisting of a protein endogenous to the compartment, a regulator protein, a motor protein, a DNA repair protein, and a combination thereof. In certain instances, the protein endogenous to the compartment is a protein localized to the compartment, a component of the compartment, a protein found within the compartment, and/or a protein associated with the compartment. In certain instances, the regulator protein is an activator or repressor of gene expression. In certain instances, the motor protein is any protein that facilitates the transport of molecules along microtubules or actin filaments. In certain instances, the DNA repair protein is any protein that repairs double-strand breaks.

In some embodiments, the compartment is a nuclear compartment (e.g., a nuclear body). In some embodiments, the nuclear compartment comprises an inner nuclear membrane and/or the compartment-specific protein comprises Emerin, Lap2beta, Lamin B, or a combination thereof. In some embodiments, the nuclear compartment comprises a Cajal body and/or the compartment-specific protein comprises coilin, SMN, Gemin 3, SmD1, SmE, or a combination thereof. In some embodiments, the nuclear compartment comprises a nuclear speckle and/or the compartment-specific protein comprises SC35. In some embodiments, the nuclear compartment comprises a PML body and/or the compartment-specific protein comprises PML, SP100, or a combination thereof. In some embodiments, the nuclear compartment comprises a nuclear core complex and/or the compartment-specific protein comprises Nup50, Nup98, Nup53, Nup153, Nup62, or a combination thereof. In some embodiments, the nuclear compartment comprises a nucleolus and/or the compartment-specific protein comprises nucleolar protein B23. In some embodiments, the nuclear compartment comprises heterochromatin and/or the compartment-specific protein comprises a regulator protein such as heterochromatin protein 1 (e.g., HP1α, HP1β, and/or HP1γ, including truncated and full-length), Krüppel-associated box-zinc finger protein (KRAB-ZFP), KRAB-associated protein 1 (KAP1), nucleosome remodeling deacetylase complex (NuRD), SET domain bifurcated 1 (SETDB1), DNA methyltransferase (e.g., DNMT3A, DNMT3L, DNMT3B), histone deacetylase (HDAC), SUV39H1 (truncated, full-length), G9a (truncated, full-length), Ezh1/2, EED, Suz12, JARID2, AEBP2, RbAp48, PCL1, RBBP7/4, C17orf96, C10orf12, or a combination thereof. In some embodiments, the nuclear compartment comprises a nuclear body and/or the compartment-specific protein comprises a DNA repair protein such as 53BP1, Rad51, Rad52, Ubc9, UBL1, BLM, c-Abl, BCR/Abl, BRCA1/2, PALB2, RPA, Rad51AP1, Chk1, Arg, Hop2, Mndl, DMC1, or a combination thereof.

In some embodiments, the compartment is a cytoplasmic compartment (e.g., a cellular body). In some embodiments, the cytoplasmic compartment comprises a P granule and/or the compartment-specific protein comprises one or more RGG domain proteins (e.g., PGL-1 and PGL-3, Dead box proteins, GLH-1-4, or a combination thereof. In some embodiments, the cytoplasmic compartment comprises a GW body and/or the compartment-specific protein comprises GW182. In some embodiments, the cytoplasmic compartment comprises a stress granule and/or the compartment-specific protein comprises G3BP (Ras-GAP SH3 binding proteins), TIA-1 (T-cell intracellular antigen), eIF2, eIF4E, or a combination thereof. In some embodiments, the cytoplasmic compartment comprises a sponge body and/or the compartment-specific protein comprises EXu, Btz, Tral, Cup, eIF4E, Me31B, Yps, Gus, Dcp1/2, Sqd, BicC, Hrb27C, Bru, or a combination thereof. In some embodiments, the cytoplasmic compartment comprises a cytoplasmic prion protein induced ribonucleoprotein (CyPrP-RNP) granule and/or the compartment-specific protein comprises Dcp1a, DDX6/Rck/p54/Me31B/Dhh1, Dicer, or a combination thereof. In some embodiments, the cytoplasmic compartment comprises a U body and/or the compartment-specific protein comprises one or more uridine-rich small nuclear ribonucleoproteins U1, U2, U4/U6 and U5; LSm1-7; the survival of motor neurons (SMN) protein, or a combination thereof. In some embodiments, the cytoplasmic compartment comprises the endoplasmic reticulum and/or the compartment-specific protein comprises Calreticulin, Calnexin, PDI, GRP 78, GRP 94, or a combination thereof. In some embodiments, the cytoplasmic compartment comprises a mitochondrium and/or the compartment-specific protein comprises HIF1A, PLN, Cox1, Hexokinase, TOMM40, or a combination thereof. In some embodiments, the cytoplasmic compartment comprises the plasma membrane and/or the compartment-specific protein comprises sodium potassium ATPase, CD98, one or more Cadherins, plasma membrane calcium ATPase (PMCA), or a combination thereof. In some embodiments, the cytoplasmic compartment comprises the Golgi apparatus and/or the compartment-specific protein comprises GM130, MAN2A1, MAN2A2, GLG1, B4GALT1, RCAS1, GRASP65, or a combination thereof. In some embodiments, the cytoplasmic compartment comprises a ribosome and/or the compartment-specific protein comprises AGO2, MTOR, PTEN, RPL26, FBL, RPS3, or a combination thereof. In some embodiments, the cytoplasmic compartment comprises a proteasome and/or the compartment-specific protein comprises PSMA1, PSMB5, PSMC1, PSMD1, PSMD7, or a combination thereof. In some embodiments, the cytoplasmic compartment comprises an endosome and/or the compartment-specific protein comprises CFTR, ADRB1, EGFR, IGF2R, AP2S1, CD4, HLA-A, Coveolin, RABS, ErbB2, or a combination thereof. In some embodiments, the cytoplasmic compartment comprises a liposome and/or the compartment-specific protein comprises EEA1, LAMTOR2, LAMTOR4, or a combination thereof. In some embodiments, the cytoplasmic compartment comprises a cytoskeletal component (e.g., microtubules and/or actin filaments) and/or the compartment-specific protein comprises a motor protein such as a kinesin, dynein, myosin, or a combination thereof.

In some embodiments, the compartment-specific protein is further linked (e.g., fused) to a fluorescent protein. In some embodiments, the actuator moiety is further linked (e.g., fused) to a fluorescent protein. In some embodiments, the first dimerization domain and the second dimerization domain comprise an inducible dimerization system that assembles to form a dimer only in the presence of a ligand, light, or an enzyme. In some embodiments, the first dimerization domain and the second dimerization domain each bind to the ligand in the presence of the ligand. In some embodiments, the ligand is a chemical inducer or an optogenetic inducer. In some embodiments, the first dimerization domain and the second dimerization domain comprise a spontaneous dimerization system.

In some embodiments, the system comprises a first polynucleotide (e.g., vector) comprising a nucleic acid sequence encoding the compartment-specific protein linked to the first dimerization domain and a second polynucleotide (e.g., vector) comprising a nucleic acid sequence encoding the actuator moiety linked to the second dimerization domain.

In another aspect, a method of controlling the spatial positioning of a target polynucleotide in a compartment of a cell is provided. The method comprises providing (e.g., introducing into the cell) a compartment-specific protein linked (e.g., fused) to a first dimerization domain. The method further comprises providing (e.g., introducing into the cell) an actuator moiety linked (e.g., fused) to a second dimerization domain. The method further comprises forming a complex comprising the actuator moiety and the target polynucleotide. The method further comprises assembling a dimer comprising the first dimerization domain and the second dimerization domain, thereby positioning the target polynucleotide in the compartment. In some embodiments, the cell is a eukaryotic cell.

In some embodiments, the target polynucleotide is not endogenous to the compartment. In some embodiments, the positioning of the target polynucleotide comprises regulating the expression of the target polynucleotide. In some embodiments, the regulating comprises decreasing the expression of the target polynucleotide. In some embodiments, the regulating comprises increasing the expression of the target polynucleotide. In some embodiments, the positioning of the target polynucleotide further comprises regulating the expression of one or more additional polynucleotides endogenous to the compartment. In some embodiments, the positioning of the target polynucleotide comprises altering cellular function, cell fate, cell growth, apoptosis, and/or cell differentiation, e.g., by repositioning the target polynucleotide (e.g., telomere) to a different cellular compartment. In certain instances, the positioning of the target polynucleotide (e.g., telomere) to a nuclear compartment such as the nuclear periphery or a Cajal body increases or decreases cell viability. In some embodiments, the positioning of the target polynucleotide further comprises creating one or more additional compartments within the cell. In some embodiments, the positioning of the target polynucleotide further comprises repairing a DNA break. In certain embodiments, the DNA break is a single-strand break or a double-strand break. In some embodiments, the repairing comprises introducing exogenous DNA. In some embodiments, the introducing comprises recombination, non-homologous end-joining (NHEJ), or homology-directed repair (HDR). In some embodiments, the positioning of the target polynucleotide induces a phase separation to form the compartment. In certain embodiments, the compartment is an artificial aggregate comprising protein, RNA, DNA, or a combination thereof. In some instances, the compartment is a nuclear body (e.g., Cajal body) or a cellular body. In some embodiments, the positioning of the target polynucleotide induces the formation of a nuclear body that facilitates DNA repair (e.g., promotes the repair of double-strand breaks) and improves gene editing efficiency (e.g., enhances HDR).

In some embodiments, the target polynucleotide comprises genomic DNA. In some embodiments, the target polynucleotide comprises RNA. In some embodiments, the actuator moiety comprises a Cas protein, and the method further comprises providing a guide RNA that complexes with the actuator moiety and hybridizes to the target polynucleotide (e.g., genomic DNA). In some embodiments, the actuator moiety comprises an RNA-binding protein, and the method further comprises providing a guide RNA that complexes with the actuator moiety and hybridizes to the target polynucleotide (e.g., RNA). In certain instances, the method further comprises providing a Cas protein that complexes with the guide RNA. In some embodiments, the RNA-binding protein is ADAR1 or ADAR2 and the guide RNA comprises an ADAR-recruiting RNA (arRNA). In some embodiments, the Cas protein substantially lacks DNA cleavage activity. In some embodiments, the Cas protein is a Cas9 protein, a Cas12 protein, a Cas13 protein, a CasX protein, or a CasY protein. In some embodiments, the Cas12 protein is selected from the group consisting of Cas12a, Cas12b, Cas12c, Cas12d, and Cas12e. In some embodiments, the Cas13 protein is selected from the group consisting of Cas13a, Cas13b, Cas13c, and Cas13d. In certain instances, the Cas13d protein is CasRx. In some embodiments, the actuator moiety comprises a binding protein that hybridizes to the target polynucleotide, wherein the binding protein is a zinc finger nuclease or a TALE nuclease. In some embodiments, the actuator moiety comprises an Argonaute protein complexed with a guide polynucleotide, wherein the guide polynucleotide is a guide RNA or a guide DNA, and wherein the guide polynucleotide hybridizes to the target polynucleotide.

In some embodiments, the compartment-specific protein is selected from the group consisting of a protein endogenous to the compartment, a regulator protein, a motor protein, a DNA repair protein, and a combination thereof. In certain instances, the protein endogenous to the compartment is a protein localized to the compartment, a component of the compartment, a protein found within the compartment, and/or a protein associated with the compartment. In certain instances, the regulator protein is an activator or repressor of gene expression. In certain instances, the motor protein is any protein that facilitates the transport of molecules along microtubules or actin filaments. In certain instances, the DNA repair protein is any protein that repairs double-strand breaks.

In some embodiments, the compartment is a nuclear compartment (e.g., a nuclear body). In some embodiments, the nuclear compartment comprises an inner nuclear membrane and/or the compartment-specific protein comprises Emerin, Lap2beta, Lamin B, or a combination thereof. In some embodiments, the nuclear compartment comprises a Cajal body and/or the compartment-specific protein comprises coilin, SMN, Gemin 3, SmD1, SmE, or a combination thereof. In some embodiments, the nuclear compartment comprises a nuclear speckle and/or the compartment-specific protein comprises SC35. In some embodiments, the nuclear compartment comprises a PML body and/or the compartment-specific protein comprises PML, SP100, or a combination thereof. In some embodiments, the nuclear compartment comprises a nuclear core complex and/or the compartment-specific protein comprises Nup50, Nup98, Nup53, Nup153, Nup62, or a combination thereof. In some embodiments, the nuclear compartment comprises a nucleolus and/or the compartment-specific protein comprises nucleolar protein B23. In some embodiments, the nuclear compartment comprises heterochromatin and/or the compartment-specific protein comprises a regulator protein such as heterochromatin protein 1 (e.g., HP1α, HP1β, and/or HP1γ, including truncated and full-length), Krüppel-associated box-zinc finger protein (KRAB-ZFP), KRAB-associated protein 1 (KAP1), nucleosome remodeling deacetylase complex (NuRD), SET domain bifurcated 1 (SETDB1), DNA methyltransferase (e.g., DNMT3A, DNMT3L, DNMT3B), histone deacetylase (HDAC), SUV39H1 (truncated, full-length), G9a (truncated, full-length), Ezh1/2, EED, Suz12, JARID2, AEBP2, RbAp48, PCL1, RBBP7/4, C17orf96, C10orf12, or a combination thereof. In some embodiments, the nuclear compartment comprises a nuclear body and/or the compartment-specific protein comprises a DNA repair protein such as 53BP1, Rad51, Rad52, Ubc9, UBL1, BLM, c-Abl, BCR/Abl, BRCA1/2, PALB2, RPA, Rad51AP1, Chk1, Arg, Hop2, Mndl, DMC1, or a combination thereof.

In some embodiments, the compartment is a cytoplasmic compartment e.g., a cellular body). In some embodiments, the cytoplasmic compartment comprises a P granule and/or the compartment-specific protein comprises one or more RGG domain proteins (e.g., PGL-1 and PGL-3, Dead box proteins, GLH-1-4, or a combination thereof. In some embodiments, the cytoplasmic compartment comprises a GW body and/or the compartment-specific protein comprises GW182. In some embodiments, the cytoplasmic compartment comprises a stress granule and/or the compartment-specific protein comprises G3BP (Ras-GAP SH3 binding proteins), TIA-1 (T-cell intracellular antigen), eIF2, eIF4E, or a combination thereof. In some embodiments, the cytoplasmic compartment comprises a sponge body and/or the compartment-specific protein comprises EXu, Btz, Tral, Cup, eIF4E, Me31B, Yps, Gus, Dcp1/2, Sqd, BicC, Hrb27C, Bru, or a combination thereof. In some embodiments, the cytoplasmic compartment comprises a cytoplasmic prion protein induced ribonucleoprotein (CyPrP-RNP) granule and/or the compartment-specific protein comprises Dcp1a, DDX6/Rck/p54/Me31B/Dhh1, Dicer, or a combination thereof. In some embodiments, the cytoplasmic compartment comprises a U body and/or the compartment-specific protein comprises one or more uridine-rich small nuclear ribonucleoproteins U1, U2, U4/U6 and U5; LSm1-7; the survival of motor neurons (SMN) protein, or a combination thereof. In some embodiments, the cytoplasmic compartment comprises the endoplasmic reticulum and/or the compartment-specific protein comprises Calreticulin, Calnexin, PDI, GRP 78, GRP 94, or a combination thereof. In some embodiments, the cytoplasmic compartment comprises a mitochondrium and/or the compartment-specific protein comprises HIF1A, PLN, Cox1, Hexokinase, TOMM40, or a combination thereof. In some embodiments, the cytoplasmic compartment comprises the plasma membrane and/or the compartment-specific protein comprises sodium potassium ATPase, CD98, one or more Cadherins, plasma membrane calcium ATPase (PMCA), or a combination thereof. In some embodiments, the cytoplasmic compartment comprises the Golgi apparatus and/or the compartment-specific protein comprises GM130, MAN2A1, MAN2A2, GLG1, B4GALT1, RCAS1, GRASP65, or a combination thereof. In some embodiments, the cytoplasmic compartment comprises a ribosome and/or the compartment-specific protein comprises AGO2, MTOR, PTEN, RPL26, FBL, RPS3, or a combination thereof. In some embodiments, the cytoplasmic compartment comprises a proteasome and/or the compartment-specific protein comprises PSMA1, PSMB5, PSMC1, PSMD1, PSMD7, or a combination thereof. In some embodiments, the cytoplasmic compartment comprises an endosome and/or the compartment-specific protein comprises CFTR, ADRB1, EGFR, IGF2R, AP2S1, CD4, HLA-A, Coveolin, RABS, ErbB2, or a combination thereof. In some embodiments, the cytoplasmic compartment comprises a liposome and/or the compartment-specific protein comprises EEA1, LAMTOR2, LAMTOR4, or a combination thereof. In some embodiments, the cytoplasmic compartment comprises a cytoskeletal component (e.g., microtubules and/or actin filaments) and/or the compartment-specific protein comprises a motor protein such as a kinesin, dynein, myosin, or a combination thereof.

In some embodiments, the compartment-specific protein is further linked (e.g., fused) to a fluorescent protein. In some embodiments, the actuator moiety is further linked (e.g., fused) to a fluorescent protein. In some embodiments, the assembling of the first and second dimerization domains is inducible and occurs only in the presence of a ligand, light, or an enzyme. In some embodiments, the first dimerization domain and the second dimerization domain each bind to the ligand in the presence of the ligand. In some embodiments, the ligand is a chemical inducer or an optogenetic inducer. In some embodiments, the first dimerization domain and the second dimerization domain comprise a spontaneous dimerization system.

In some embodiments, the method comprises introducing into the cell a system described herein comprising a first polynucleotide (e.g., vector) comprising a nucleic acid sequence encoding the compartment-specific protein linked to the first dimerization domain and a second polynucleotide (e.g., vector) comprising a nucleic acid sequence encoding the actuator moiety linked to the second dimerization domain.

### BRIEF DESCRIPTION OF THE DRAWINGS

FIG. 1 is a schematic illustration of a programmable, inducible, and versatile system for targeting genomic loci to various nuclear compartments. dCas9 and a nuclear compartment-specific protein are fused to complementary pairs of heterodimerization domains, which assemble only in the presence of a chemical inducer. The genomic targets are specified by the sgRNA sequences, and nuclear compartments are programmed by fusing CRISPR-GO with compartment-specific molecules.

FIG. 2 is a schematic illustration of an abscisic acid (ABA)-inducible CRISPR-GO system to target genomic loci to the nuclear envelope (NE) through co-expression of ABI-dCas9 and PYL1-GFP-Emerin in human cells. In the presence of ABA, ABI and PYL1 dimerize, causing relocalization of ABI-dCas9-targeted genomic loci to PYL1-GFP-Emerin at the nuclear envelope. After removal of ABA, ABI and PYL1 dissociate and genomic loci are no longer tethered to the NE.

FIG. 3 is a schematic illustration of the ABA-inducible CRISPR-GO system with co-expression of ABI-BFP-dCas9 and PYL1-GFP-Emerin in human cells. ABA treatment dimerizes ABI and PYL1 and re-localizes ABI-BFP-dCas9-targeted genomic loci to the nuclear periphery containing PYL1-GFP-Emerin.

FIG. 4 is a schematic illustration of the TMP-HTag inducible CRISPR-GO system with co-expression of dCas9-EGFP-HaloTag and DHFR-Emerin-mCherry in human cells. TMP-HTag treatment dimerizes DHFR and HaloTag and re-localizes dCas9-EGFP-HaloTag-targeted genomic loci to the nuclear periphery containing DHFR-Emerin-mCherry.

FIG. 5 is a schematic illustration of the method to use CRISPR-Cas9 imaging to visualize repetitive genomic loci targeted by the CRISPR-GO system in living cells. Both AB1-dCas9 and dCas9-HaloTag bind to the same repetitive genomic locus. While AB1-dCas9 dimerizes with PYL1-Emerin to re-localize the genomic locus, dCas9-HaloTag binds to cell permeable JF549-HaloTag dye ligand to enable visualization of the targeted genomic locus in living cells.

FIG. 6 presents representative microscopic images of U2OS cells showing co-expression of AB1-BFP-dCas9, PYL1-GFP-Emerin, and dCas9-HaloTag, without sgRNAs. AB1-BFP-dCas9 likely accumulate in nucleoli without ABA treatment. ABA treatment-induced heterodimerization relocated AB1-BFP-dCas9 to the nuclear envelope (NE) and Endoplasmic Reticulum (ER), as marked by PYL1-GFP-Emerin. dCas9-HaloTag had a low expression level and was evenly distributed throughout the nucleus; its location remained unaffected by ABA treatment. Scale bars, 10 μm.

FIG. 7 is a summary of chromosome locations of highly repetitive regions targeted by CRISPR-GO in FIGS. 8 and 9. A single sgRNA binds to multiple repeats (solid grey boxes) within the targeted regions. The genes adjacent to the targeted site are shown in italic letters in grey-outlined boxes.

FIG. 8 presents graphs of the quantification of CRISPR-GO-induced genomic repositioning efficiency of highly repetitive genomic loci. Chr3, Chr13, and LacO loci are labeled using CRISPR-Cas9 imaging in living cells. Telomeres are labeled by a telomere marker, TRF1-mCherry. The nuclear envelope is visualized by GFP-Emerin. For each locus, the left bar graph shows the percentage of genomic loci at the nuclear periphery, and the right bar graph shows the percentage of cells containing at least one nuclear periphery-associated locus. The numbers of loci and cells analyzed are on the bottom.

FIG. 9 presents graphs of the quantification of CRISPR-GO-induced nuclear repositioning efficiency of less repetitive endogenous genomic loci. Genomic loci were visualized by 3D-FISH and nuclei are stained by DAPI. For each locus, the left bar graph shows the percentage of genomic loci at the nuclear periphery, and the right bar graph shows the percentage of cells containing at least one nuclear periphery-associated locus. The numbers of loci and cells analyzed are on the bottom.

FIG. 10 presents representative microscopy images comparing the localization of targeted genomic loci (arrows) labeled by CRISPR-Cas9 imaging with or without ABA. PYL1-GFP-Emerin is shown localized to the nuclear envelope (NE) and endoplasmic reticulum (ER). The nuclear periphery is outlined by dotted white lines except for regions next to tethered genomic loci. Insets show enlarged images of periphery-tethered genomic loci. Scale bars, 10 μm.

FIG. 11 presents individual channels of the representative microscopic images in FIG. 10 comparing the localization of targeted genomic loci (arrows) and nuclear periphery (dotted lines) with or without ABA. The top row shows PYL1-GFP-Emerin that is localized to the nuclear envelope (NE) and endoplasmic reticulum (ER). The nuclear periphery is outlined by dotted white lines (bottom) except for regions next to tethered genomic loci. Scale bars, 10 μm.

FIG. 12 presents graphs of linescans of the fluorescence intensity of labeled Chr3 loci and labeled PYL1-GFP-Emerin without (top) and with ABA treatment (bottom) along the dotted lines as shown in the Emerin images at the top of FIG. 11. Chr3 loci are labeled by CRISPR-Cas9 imaging through the addition of the JF549-halotag dye.

FIG. 13 presents graphs of linescans of the fluorescence intensity of labeled LacO loci (FISH, Alexa646) and labeled nucleus (DAPI) without (top) and with ABA treatment (bottom) along the dotted lines as shown.

FIG. 14 is a summary of chromosome locations of less repetitive regions targeted by CRISPR-GO in FIGS. 8 and 9. A single sgRNA binds to multiple repeats (solid grey boxes) within the targeted regions. The genes adjacent to the targeted site are shown in italic letters in grey-outlined boxes.

FIG. 15 presents representative microscopy images comparing the localization of targeted genomic loci (arrows) labeled by 3D-FISH with or without ABA. Nuclei labeled by DAPI are shown. The nuclear periphery is outlined by dotted white lines except for regions next to tethered genomic loci. Insets show enlarged images of periphery-tethered genomic loci. See FIG. 11 for individual channels. Scale bars, 10 μm.

FIG. 16 presents graphs of quantification of percentages of nuclear periphery localized genomic loci (Chr7, ChrX, and CXCR4) in CRISPR-GO cells transfected with a non-targeting sgRNA. For each locus, the left bar graph shows the percentages of the nuclear periphery localized genomic loci, and the right bar graph shows the percentages of cells containing at least one periphery-associated locus.

FIG. 17 presents a summary of chromosome locations of non-repetitive regions targeted by CRISPR-GO in FIGS. 18 and 19. Multiple sgRNAs are designed to tile along the regions upstream or within the gene bodies of the targeted genes (XIST, PTEN, CXCR4). The sgRNA-targeted regions are shown in solid grey boxes. The top grey boxes show sgRNA targets within the forward strand and bottom grey boxes show sgRNAs targets within the reverse strand. The genes adjacent to the targeted site are shown in in italic letters in grey boxes.

FIG. 18 presents graphs of quantification of CRISPR-GO-induced nuclear repositioning efficiency of non-repetitive endogenous genomic loci. The non-repetitive locus adjacent to CXCR4 was targeted with a single sgRNA or multiple sgRNAs pooled together. Genomic loci were visualized by 3D-FISH and nuclei are stained by DAPI. For each locus, the left bar graph shows the percentage of genomic loci at the nuclear periphery, and the right bar graph shows the percentage of cells containing at least one nuclear periphery-associated locus. The numbers of loci and cells analyzed are on the bottom.

FIG. 19 presents graphs of a comparison of re-localization efficacy targeting CXCR4 loci using single sgRNAs (sgCXCR4-1, left; sgCXCR4-2, middle) or 6 sgRNAs (right). For each locus, the left bar graph shows the percentage of genomic loci at the nuclear periphery, and the right bar graph shows the percentage of cells containing at least one nuclear periphery-associated locus. The numbers of loci and cells analyzed are on the bottom.

FIG. 20 is a graph of the time course of the inducible and reversible repositioning of endogenous locus Chr3:q29, mediated by addition or removal of ABA. The Y axis shows the percentage of periphery-localized Chr3:q29 loci. The X axis shows the time in hours from ABA addition or removal. Data are represented as mean±SEM.

FIG. 21 is a graph of a comparison of the genomic repositioning efficacy in S-phase arrested cells (+ABA, +HU) and control cells (+ABA, −HU) at different time points after ABA addition. The Y axis shows the percentage of periphery-localized Chr3:q29 loci at different time points. Data are represented as mean±SEM. The box on the left shows the outline of the time-course experiment.

FIG. 22 presents representative microscopy images showing mitosis-independent tethering of endogenous Chr3:q29 loci (arrow) to the nuclear envelope. A Chr3:q29 locus (arrow) starts off separate from the nuclear envelope in the first 4 h of recording. Nuclear periphery tethering occurs at 4.5 h and remains stable for the rest of the 8 h of recording. Images here are insets in FIG. 23. Scale bar, 2 μm.

FIG. 23 presents representative microscopic images showing mitosis-independent tethering of endogenous genomic loci to the nuclear periphery. The insets are also shown in FIG. 22. PYL1-GFP-Emerin is localized to the nuclear envelope (NE) and the endoplasmic reticulum (ER), and the nuclear envelope is outlined by dotted lines. A Chr3 locus is not adjacent to the nuclear envelope in the first 4 h of recording. Nuclear periphery tethering happens at 4.5 h and remains for the rest of the 8 h of recording. Nuclear rotation happens between 10 h and 12 h. Scale bar, 10 μm.

FIG. 24 is a graph showing the distances between the genomic locus in FIG. 22 and nearest nuclear periphery at different time points. Images were taken every 30 mins.

FIG. 25 presents scatter plots of step displacement (dx, dy) of untethered (1&2) and tethered (3&4) Chr3 loci. The step displacement is calculated by subtracting the position of a previous time point from the new position: dx^{t}=(x^{t}−x^{t-1}) and dy^{t}=y^{t}−y^{t-1}. Movements were tracked every 4 s for 6 min.

FIG. 26 is a graph of the comparison of average step distance of untethered (1696 steps in 19 cells) and tethered (1669 steps in 14 cells) Chr3:q29 loci. p<0.0001 by a two-side t-test with unequal variance. Data are represented as mean±SD.

FIG. 27 is a graph of the fitting of the step distances of untethered and tethered Chr3:q29 loci using gamma distribution. Fitted parameters: shape parameter k=2.4 for untethered loci and 1.9 for tethered loci; rate parameter (3=21.9 for untethered loci and 46.3 for tethered loci.

FIG. 28 is a schematic illustration of an ABA-inducible CRISPR-GO system to target genomic loci to CBs through co-expression of ABI-dCas9 and PYL1-GFP-Coilin in human cells. ABA treatment dimerizes ABI and PYL1 and tethers ABI-dCas9-targeted genomic loci to CBs containing PYL1-GFP-Coilin.

FIG. 29 presents representative microscopic images showing the colocalization of the targeted LacO loci (top panels, by FISH) and Coilin-GFP-labeled CBs (middle panels) with or without ABA.

FIG. 30 presents graphs of quantification of CRISPR-GO-induced CB tethering efficiency of LacO loci. The left bar graph shows the percentage of LacO loci that co-localize with Coilin-GFP labeled CBs, and the right bar graph shows the percentage of cells containing at least one CB-colocalized LacO locus. The number of loci and cells analyzed are labeled on the bottom. Data are represented as mean±SEM.

FIG. 31 presents representative microscope images showing the colocalization of other CB components (SMN, Fibrillarin, Gemin2, by immunostaining) with LacO loci (by FISH) using the CRISPR-GO system to tether LacO loci to CBs.

FIG. 32 presents representative microscopic images showing colocalization of targeted Chr3:q29 loci (top panels, by CRISPR-Cas9 imaging) and Coilin-GFP labeled CBs (middle panels) with or without ABA.

FIG. 33 presents graphs of quantification of CRISPR-GO induced CB-tethering efficiency of Chr3:q29 loci. The left bar graph shows the percentage of Chr3:q29 loci that co-localize with CBs, and the right bar graph shows the percentage of cells containing at least one CB-colocalized Chr3:q29 locus. The numbers of loci and cells are on the bottom. Data are represented as mean±SEM.

FIG. 34 is a schematic illustration of an ABA-inducible CRISPR-GO system to target genomic loci to PML bodies through co-expression of ABI-dCas9 and PYL1-GFP-PML.

FIG. 35 presents representative microscopic images showing colocalization of targeted Chr3:q29 loci (top panels, by CRISPR-Cas9 imaging) and PML-GFP labeled PML bodies (middle panels) with or without ABA.

FIG. 36 presents graphs of quantification of CRISPR-GO-induced PML body tethering efficiency to the targeted Chr3:q29 loci. The left bar graph shows the percentage of Chr3:q29 loci that colocalize with PML bodies, and the right bar graph shows the percentage of cells containing at least one PML body-colocalized Chr3:q29 locus. The numbers of loci and cells are on the bottom. Data are represented as mean±SEM.

FIG. 37 presents representative microscopic images showing colocalization of another PML body marker, SP100 (immunostaining), with Chr3:q29 loci (by CRISPR-Cas9 imaging) after using CRISPR-GO to tether Chr3:q29 loci to PML bodies. Scale bars, 10 μm.

FIG. 38 is a graph of rapidly inducible chromatin-CBs association through addition of ABA. The Y axis shows the percentage of CB-colocalized LacO loci. Data are represented as mean±SEM.

FIG. 39 is a plot diagram showing dynamics of chromatin-CBs disassociation after removal of ABA. The Y axis shows the percentage of CB-colocalized LacO loci. X axis shows the time in hours from ABA removal. Data are represented as mean±SEM.

FIG. 40 presents a comparison of GFP-Coilin fluorescence at targeted LacO loci in cells treated with ABA (top) and 6 hours after ABA removal (bottom two rows). Two representative microscopic images are shown for cells with dimmed CBs (middle) or cells in which GFP-Coilin CBs have disappeared (bottom). Linescan (right) measures the raw fluorescence intensity of GFP-Coilin and LacO loci along the dotted lines shown on the left.

FIG. 41 presents representative real-time microscopic images showing the rapid formation of a de novo CB (Coilin) at the targeted LacO locus mediated by CRISPR-GO. The chosen cell was imaged first before ABA treatment (−150 s). ABA was added to the culture medium between −150 s and 0 s, and 1 s represents the first image taken of the same cell immediately after ABA addition.

FIG. 42 shows repression of endogenous gene expression adjacent to targeted loci and across long distances by Cajal body colocalization. Left: schematic illustration of the CRISPR-GO system to colocalize the Chr3:q29 locus to CBs in U2OS cells. ACAP2 is located ˜35 kb upstream of the sgRNA target site, and PPP1R2 is located ˜36 kb downstream of the sgRNA target site. Right: Graph of comparison of ACAP2 and PPP1R2 gene expression (measured by RT-qPCR) using CRISPR-GO to colocalize Chr3:q29 loci to CBs in +/−ABA conditions. See FIG. 43 for controls.

FIG. 43 presents graphs of controls for using CRISPR-GO to colocalize the endogenous Chr3 loci with CBs. Left: measurement of ACAP2 and PPP1R2 mRNA expression with the CRISPR-GO system but without a targeting sgRNA with and without ABA; Right: measurement of ACAP2 and PPP1R2 mRNA expression with ABI-dCas9 and a Chr3-targeting sgRNA, but without PYL1-GFP-Coilin with and without ABA. mRNA was measured using RT-qPCR under different conditions.

FIG. 44 is a graph of quantification of the Coilin-GFP fluorescence intensity at the targeted LacO loci shown in FIG. 41. The fluorescence intensity before ABA addition at −150 s was set to 0 (background).

FIG. 45 presents real-time microscopic images showing colocalization of an existing CB (Coilin, arrow) to an adjacent targeted LacO locus mediated by CRISPR-GO. The chosen cell was imaged before ABA treatment (−200 s). ABA was added to the culture medium between −200 s and 0 s, and 0 s represents the first image taken immediately after ABA addition. Scale bars, 10 μm.

FIG. 46 shows adjacent reporter gene expression repressed by repositioning targeted chromatin DNA to the nuclear periphery. Left: schematic illustration of the CRISPR-GO system to reposition a LacO repeat array to the nuclear periphery in the U2OS 2-6-3 cells, which is inserted adjacent to a Doxycycline (Dox)-inducible TRE-miniCMV promoter driving a CFP-SKL reporter gene. Right: graph of comparison of CFP reporter expression level using the CRISPR-GO system to reposition LacO loci to the nuclear periphery in +/−Dox and +/−ABA conditions. Data are represented as mean±SD. See FIG. 47 for representative histograms and controls.

FIG. 47 presents representative flow cytometry histograms comparing the fluorescence intensity of CFP reporter expression using CRISPR-GO tethering of LacO loci to the nuclear periphery under different treatments. The statistics diagram is shown in FIG. 46. The right diagram shows the quantification of relative CFP fluorescence with a non-targeting sgRNA with or without ABA treatment for +/−Dox. Data are represented as mean±SDs.

FIG. 48 presents graphs of the comparison of ACAP2 and PPP1R2 gene expression when using the CRISPR-GO system to reposition Chr3 loci to the nuclear periphery. mRNA was measured using RT-qPCR under different conditions. Cells transfected with a non-targeting sgRNA (sgNT) were used as control. Data are represented as mean±SD.

FIG. 49 shows reporter gene expression adjacent to targeted loci repressed by Cajal body colocalization. Left: schematic illustration of the CRISPR-GO system to colocalize the LacO repeat array to CBs in the U2OS 2-6-3 cells. Right: graph of comparison of CFP reporter expression using the CRISPR-GO system to colocalize LacO loci to CBs for +/−Dox and +/−ABA conditions. See FIG. 50 for representative histograms and controls.

FIG. 50 presents representative flow cytometry histograms comparing the fluorescence intensity of CFP reporter expression using CRISPR-GO tethering LacO loci to CBs under different treatments. The statistics diagram is shown in FIG. 49. The right diagram shows the quantification of relative CFP fluorescence with a non-targeting sgRNA with or without ABA treatment for +/−Dox. With a non-targeting sgRNA, ABA treatment leads to slight but insignificant decrease (p>0.05) in CFP reporter expression. Data are represented as mean±SDs.

FIG. 51 presents histograms of distances between telomeres and the nearest nuclear envelope point during interphase in example cells treated with or without ABA.

FIG. 52 is a graph of the comparison of relative cell viability as measured by an Alamar blue assay after using the CRISPR-GO system to reposition telomeres to the nuclear envelope. Data are represented as mean±SD.

FIG. 53 shows a cell cycle analysis of cells using CRISPR-GO to reposition telomeres to the nuclear periphery. Cells were treated with ABA for 3 days. Top: representative flow cytometry histograms (left) compare the fluorescence Hoechst 33342 stained DNA components in telomere-targeting ABA treated cells (treated) and non-targeting sgRNA control cells. Bottom: quantification of three experimental replicates. Data are represented as mean±SDs.

FIG. 54 presents representative microscopic images of U2OS cells using CRISPR-GO to colocalize telomeres (TRF1-mCherry, top) and CBs (GFP-Coilin, middle) with or without ABA. Scale bars, 10 μm.

FIG. 55 presents representative microscopic images of HeLa cells using CRISPR-GO to colocalize telomeres (TRF1-mCherry, top) and CBs (GFP-Coilin, middle) with or without ABA. Scale bars, 10 μm.

FIG. 56 is a graph of the comparison of relative U2OS cell viability as measured by an Alamar blue assay using the CRISPR-GO system for targeting telomeres to CBs with or without ABA. Cells were treated with ABA for two days. Data are represented as mean±SD.

FIG. 57 is a graph of the comparison of relative cell viability as measured by an Alamar blue assay of U2OS cells with or without ABA. Cells were treated with ABA for two days. Data are represented as mean±SD.

FIG. 58 shows the CRISPR-GO system enabling programmable control of 3D genome organization relative to other nuclear compartments, thus expanding the CRISPR-Cas toolbox for genome engineering. The CRISPR-GO method allows for programmable control of the 3D genomic positioning and organization of targeted chromatin loci relative to diverse nuclear compartments. This expands the utility of the CRISPR-Cas toolbox beyond applications such as gene editing, transcriptional regulation, epigenetic modification.

FIG. 59 is a schematic illustration of an ABA-inducible CRISPR-GO system to target genomic loci to heterochromatin through co-expression of ABI-dCas9 and PYL1-GFP-HP1α in human cells. Also presented are representative microscopic images showing that ABA treatment dimerizes ABI and PYL1 and colocalizes ABI-dCas9-targeted genomic loci to PYL1-GFP-HP1α. Scale bars, 10 μm.

FIG. 60 is a graph of the distribution of repetitive sequences (four or more) for each human chromosome and their relative coordinates.

FIG. 61 is a graph of a genome-wide bioinformatics analysis revealing the percentage of human genes located within a given distance to adjacent repetitive sequences.

FIG. 62 shows an overview of the CRISPR-GO system 3D genome organization platform.

FIG. 63 presents a graph comparing the gene expression changes by RNA sequencing after repositioning telomeres to the nuclear periphery.

FIG. 64 presents a graph comparing the gene expression changes by RNA sequencing after co-localizing telomeres with Cajal bodies.

FIGS. 65A-65C show that CRISPR editing recruiting DNA repair components (e.g., 53BP1) creates a nuclear body that facilitates DNA repair and better gene editing outcomes.

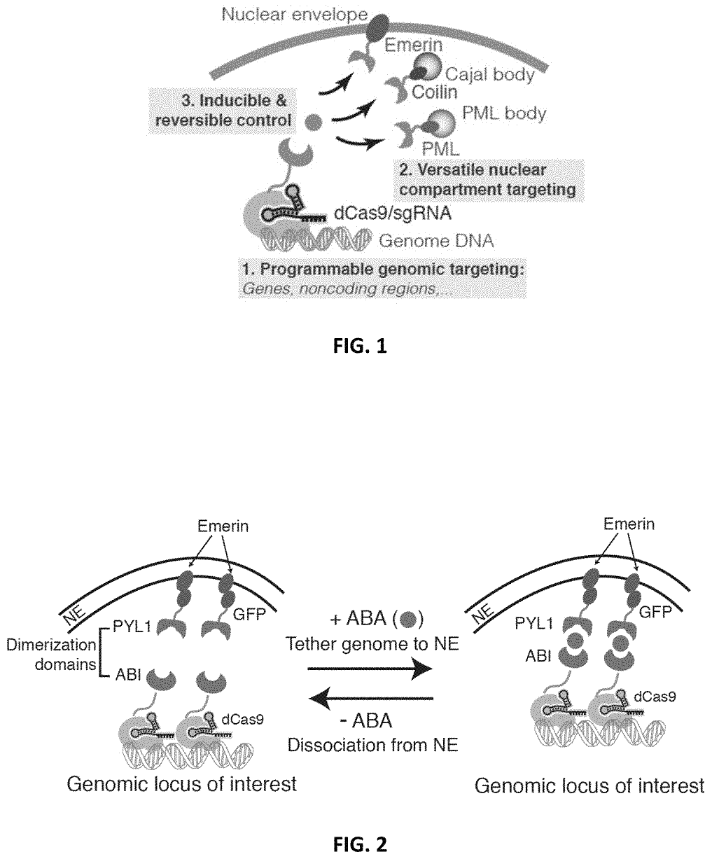

### DETAILED DESCRIPTION

### I. Introduction

Eukaryotic cells are complex structures capable of coordinating numerous biochemical reactions in space and time. Key to such coordination are both the 3D organization of polynucleotides such as the genome, and the subdivision of intracellular space into functional compartments. Compartmentalization can be achieved by intracellular membranes, which surround organelles and act as physical barriers. In addition, cells have developed sophisticated mechanisms to partition their inner substance in a tightly regulated manner. Recent studies provide compelling evidence that membraneless compartmentalization can be achieved by liquid demixing, a process culminating in liquid-liquid phase separation and the formation of phase boundaries.

The inventors have surprisingly discovered versatile systems and methods that can efficiently control the spatial positioning of polynucleotides relative to the functional compartments, including nuclear compartments such as the nuclear periphery, Cajal bodies, and promyelocytic leukemia (PML) bodies. The systems and methods can also be useful in generating synthetic phase separations, by forming supramolecular assemblies of proteins, RNA, and/or DNA molecules organized or portioned within a cell. The systems and methods disclosed herein can be useful for manipulating the spatiotemporal organization of genomic DNA and RNA components in the nucleus/cytoplasm and for regulating diverse cellular functions. The provided systems and methods also can be used for programmable control of spatial genome organization, and for applying this organization to affect polynucleotide regulation and cellular function, and to mediate interacting dynamics between targeted polynucleotides and different cellular compartments. The disclosed systems can be used, for example, to achieve the dynamic reorganization of subcellular space as a framework to manipulate pathological protein assembly in diseases including cancer and neurodegeneration.

The disclosed systems can be chemically inducible and reversible, enabling interrogation of real-time dynamics of, for example, chromatin interactions with nuclear compartments in living cells. As further examples, inducible repositioning of genomic loci to the nuclear periphery can allow dissection of mitosis-dependent and -independent relocalization events, interrogation of the relationship between gene position and expression, and understanding of the effects of telomere repositioning on cell growth. The systems described herein can mediate rapid de novo formation of Cajal bodies at target chromatin loci and causes significant repression of adjacent endogenous gene expression across long distances (>30 kb). The provided system thus offers a novel platform to investigate large-scale spatial polynucleotide organization and function in a targeted manner.

In some embodiments, the use of different sgRNAs allows the system to be programmed to flexibly target different genomic sequences. The repositioning of genomic loci to the nuclear periphery can be enabled in both mitosis-dependent and -independent manners. Target DNA colocalization with Cajal bodies can be triggered through rapid de novo Cajal body formation or through repositioning target DNA to existing Cajal bodies. Targeting genomic loci to the nuclear periphery or to Cajal bodies using the provided systems and methods can also repress adjacent reporter gene expression. Importantly, colocalization of genomic loci with Cajal bodies also can repress expression of adjacent endogenous genes (>30 kb). Furthermore, the sequestering of telomeres to the nuclear periphery using aspects of the present disclosure can negatively impact cell growth.

### II. Definitions

As used herein, the following terms have the meanings ascribed to them unless specified otherwise.

As used in the specification and claims, the singular forms “a,” “an,” and “the” include plural references unless the context clearly dictates otherwise. For example, the term “a cell” includes a plurality of cells.

The term “compartment” refers to a cellular compartment including membrane enclosed regions surrounded by a single or double lipid layer membrane and membraneless regions such as nuclear bodies and cell bodies achieved by phase separation and the formation of phase boundaries. Compartments include nuclear compartments and cytoplasmic compartments. Non-limiting examples of nuclear compartments include the nuclear periphery, the inner nuclear membrane, the nuclear pore complex, and heterochromatin, as well as nuclear bodies such as, e.g., Cajal bodies, promyelocytic leukemia (PML) bodies, nuclear speckles, and the nucleolus. Non-limiting examples of cytoplasmic compartments include membrane-bound and non-membrane-bound organelles, e.g., mitochondria, chloroplasts, peroxisomes, lysosomes, the endoplasmic reticulum, the Golgi apparatus, vesicles, vacuoles, lysosomes, endosomes, ribosomes, proteasomes, centrioles, and the cytoskeleton, as well as cellular bodies such as, e.g., P granules, GW bodies, stress granules, sponge bodies, CyPrP-RNP granules, and U bodies.

The term “compartment-specific protein” refers to a protein that is capable of positioning a target polynucleotide in a compartment, inducing or modulating the formation or localization of a compartment comprising a target polynucleotide, and/or delivering a target polynucleotide to a specific location within a cell. Compartment-specific proteins that position a target polynucleotide in a compartment are generally endogenous components of that compartment. Compartment-specific proteins that induce or modulate the formation or localization of a compartment comprising a target polynucleotide are generally regulator proteins such as gene activators or repressors. Compartment-specific proteins that deliver a target polynucleotide to a specific location within a cell are generally motor proteins or proteins involved in intracellular transport.

As used herein, a “cell” can generally refer to a biological cell. A cell can be the basic structural, functional and/or biological unit of a living organism. A cell can originate from any organism having one or more cells. Some non-limiting examples include: a prokaryotic cell, a eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Sometimes a cell is not originating from a natural organism (e.g., a cell can be a synthetically made, sometimes termed an artificial cell).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “target polynucleotide” refers to a polynucleotide or nucleic acid which is targeted by an actuator moiety of the present disclosure. A target polynucleotide can be DNA. A target polynucleotide can be RNA. A target polynucleotide can refer to a chromosomal sequence or an extrachromosomal sequence (e.g., an episomal sequence, a minicircle sequence, a mitochondrial sequence, a chloroplast sequence, etc.). A target polynucleotide can be a nucleic acid sequence that may not be related to any other sequence in a nucleic acid sample by a single nucleotide substitution. A target polynucleotide can be a nucleic acid sequence that may not be related to any other sequence in a nucleic acid sample by at least 2, 3, 4, 5, 6, 7, 8, 9, or 10 nucleotide substitutions. In some embodiments, the substitution may not occur within 5, 10, 15, 20, 25, 30, or 35 nucleotides of the 5′ end of a target polynucleotide. In some embodiments, the substitution may not occur within 5, 10, 15, 20, 25, 30, 35 nucleotides of the 3′ end of a target polynucleotide. In general, the term “target sequence” refers to a nucleic acid sequence on a single strand of a target polynucleotide. The target sequence can be a portion of a gene, a regulatory sequence, genomic DNA, cell free nucleic acid including cfDNA and/or cfRNA, cDNA, a fusion gene, and RNA including mRNA, miRNA, rRNA, and others.

The term “actuator moiety” as used herein refers to a moiety which can regulate expression or activity of a gene and/or edit a nucleic acid sequence, whether exogenous or endogenous. An actuator moiety can regulate expression of a gene at the transcription level and/or the translation level. An actuator moiety can regulate gene expression at the transcription level, for example, by regulating the production of mRNA from DNA, such as chromosomal DNA or cDNA. In some embodiments, an actuator moiety recruits at least one transcription factor that binds to a specific DNA sequence, thereby controlling the rate of transcription of genetic information from DNA to mRNA. An actuator moiety can itself bind to DNA and regulate transcription by physical obstruction, for example, by preventing proteins such as RNA polymerase and other associated proteins from assembling on a DNA template. An actuator moiety can regulate expression of a gene at the translation level, for example, by regulating the production of protein from an mRNA template. In some embodiments, an actuator moiety regulates gene expression by affecting the stability of an mRNA transcript. In some embodiments, an actuator moiety regulates expression of a gene by editing a nucleic acid sequence (e.g., a region of a genome). In some embodiments, an actuator moiety regulates expression of a gene by editing an mRNA template. Editing a nucleic acid sequence can, in some cases, alter the underlying template for gene expression.

A Cas protein referred to herein can be any type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon optimized homologue of a Cas protein. A Cas protein can be enzymatically inactive, partially active, constitutively active, fully active, inducibly active and/or more active (e.g., more than the wild-type homologue of the protein or polypeptide.). A Cas protein can be Cas9. A Cas protein can be Cas12a (Cpf1). A Cas protein can be Cas13a (C2c2). A Cas protein (e.g., variant, mutated, enzymatically inactive and/or conditionally enzymatically inactive site-directed polypeptide) can bind to a target polynucleotide. The Cas protein (e.g., variant, mutated, enzymatically inactive and/or conditionally enzymatically inactive endoribonuclease) can bind to a target RNA or DNA.

Proteins or polypeptides described herein can be “linked” to each other by a linker (e.g., a peptide or polypeptide linker) or by a peptide bond. Peptide or polypeptide linkers may contain natural amino acids, unnatural amino acids, or a combination thereof. In some embodiments, the peptide or polypeptide linker may be a flexible linker, e.g., containing amino acids such as Gly, Asn, Ser, Thr, Ala, and the like. Such linkers are designed using known parameters and may be of any length and contain any number of repeat units of any length (e.g., repeat units of Gly and Ser residues). For example, the linker may have repeats, such as two, three, four, five, or more Gly₄-Ser repeats or a single Gly₄-Ser.

### III. CRISPR-GO System

The CRISPR-Cas system has been repurposed as a flexible genome engineering platform, and has been used for applications such as gene editing, transcriptional regulation, epigenetic modifications, DNA looping, and genome imaging. Provided herein are further expansions to the CRISPR-Cas toolbox in the form of a polynucleotide organization system which enables programmable control of targeted polynucleotide positioning within the cellular compartments. In certain aspects, the targeted polynucleotides comprise genomic DNA and the system is referred to as CRISPR-GO (FIG. 58), wherein GO refers to Genome Organization. The systems and methods disclosed herein can efficiently target polynucleotides (e.g., endogenous genomic loci) to various cellular compartments (e.g., the nuclear periphery, Cajal bodies, and PML bodies). The provided systems can be inducible and reversible, allowing for the interrogation of, for example, the interaction dynamics between targeted chromatin DNA and nuclear compartments. Using this feature, both mitosis-dependent and -independent repositioning of genomic loci to the nuclear periphery have been achieved, and both de novo formation of Cajal bodies at the target loci and colocalization of existing Cajal bodies with targeted chromatin loci have been demonstrated. Colocalization of the genomic loci with the nuclear periphery or Cajal bodies using the systems and methods disclosed herein has been used to affect adjacent gene expression. Notably, colocalization of an endogenous locus with Cajal bodies using the provided systems and methods can significantly repress nearby gene expression, even though these genes are far away (>30 kb) from the target site. Finally, it has been found that repositioning telomeres to the nuclear periphery with the systems and methods disclosed herein can disrupt telomere dynamics and reduces cell viability. The provided methods offer a platform for the programmable control of polynucleotide (e.g., genomic DNA) interactions with various cellular (e.g., nuclear) compartments, which can facilitate a deeper understanding of the functional role of spatiotemporal polynucleotide organization in regulation, stability, and cellular function.

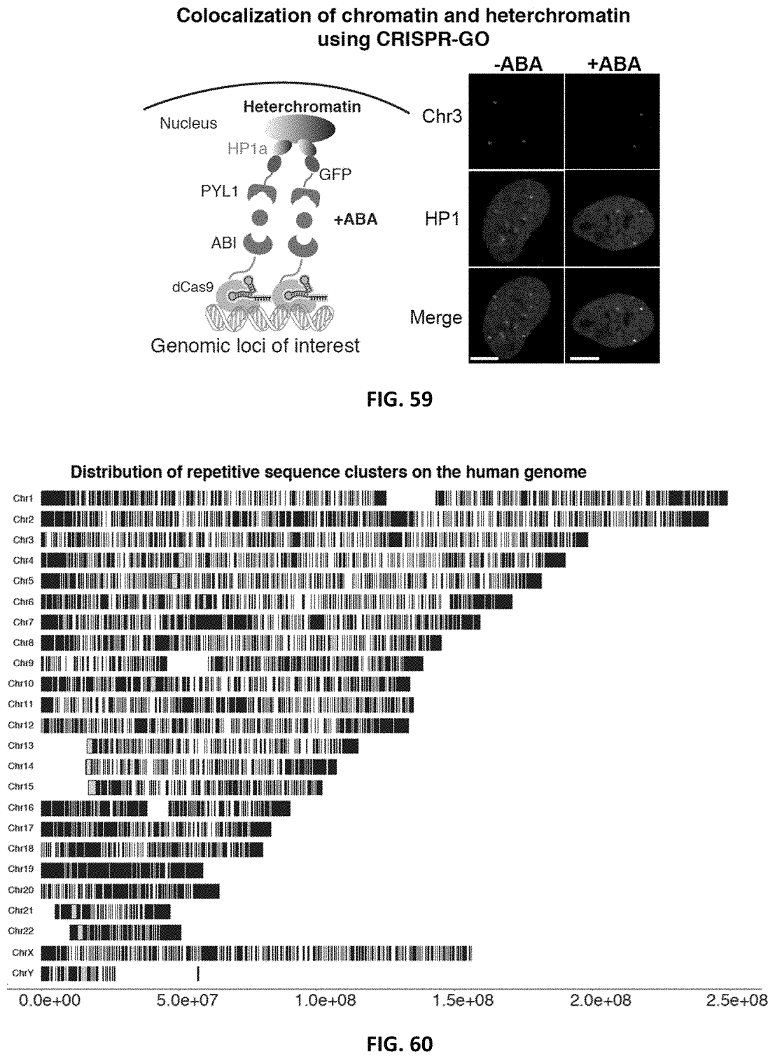

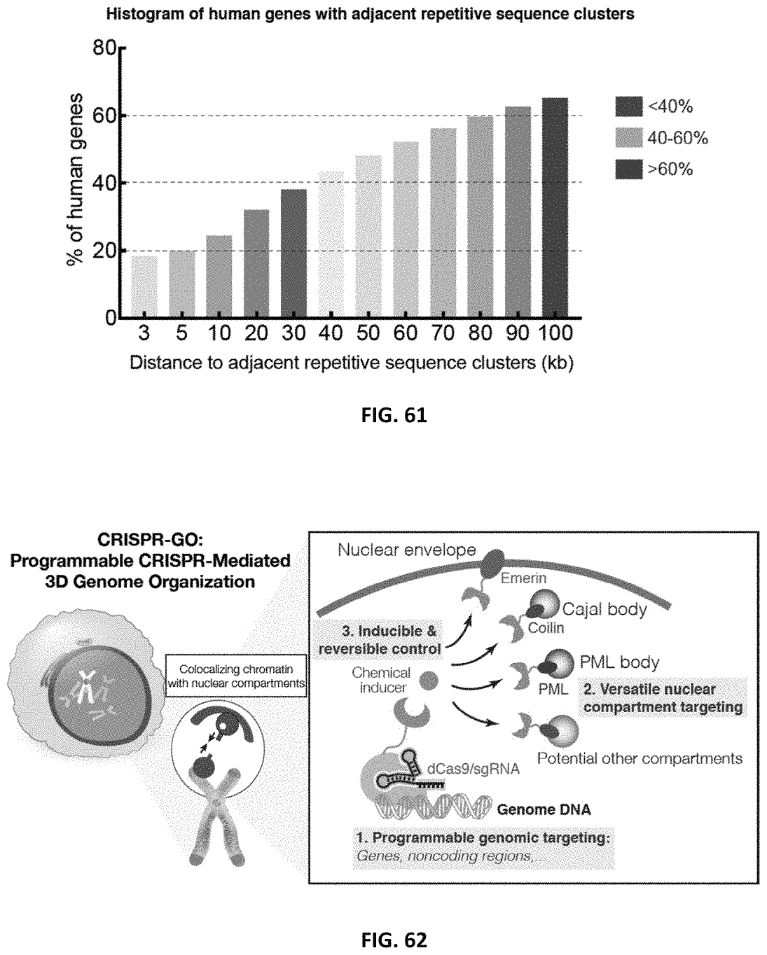

A major goal in cell biology is the understanding of how genomic interactions with different nuclear compartments affect gene expression, chromatin conformation, and cellular functions. The CRISPR-GO system can efficiently target specific genomic loci to the nuclear periphery, Cajal bodies, and PML bodies, and also holds potential to be expanded to other nuclear compartments such as nucleoli, nuclear pore complexes, and nuclear speckles. Targeting genomic loci to other nuclear compartments can be achieved by coupling CRISPR-GO with different compartment-specific proteins, such as heterochromatin protein 1α (HP1α) (FIG. 59). Similarly, the systems and methods disclosed herein provide a versatile modular platform that can be applied to the study of various cellular compartments.

The provided systems (e.g., CRISPR-GO) allow programmable re-localization of polynucleotides (e.g., genomic loci) in a precise and targeted manner. For example, the CRISPR-GO system can efficiently target repetitive and non-repetitive chromatin loci located on different chromosomes to nuclear compartments. Unlike the LacI-LacO system, the genomic targets of the CRISPR-GO system can be flexibly defined by the base-pairing interactions between sgRNAs and the target DNA sequence, and simply altering a ˜20 nt region on the sgRNAs allows for the targeting of a different genomic locus. This programmable feature can allow one to use CRISPR-GO to target a variety of genomic elements, including protein-coding genes, non-coding RNA genes, and regulatory elements. In contrast, the LacO-LacI technique is not suitable for programmable genomic targeting, as it can only be performed on well-characterized cell lines containing a highly repetitive LacO array. Creating and characterizing a useful LacO-containing cell line is difficult and laborious. LacO arrays are usually randomly inserted into the genome, after which cells containing a single-copy insertion are selected to build stable cell lines before the precise genome integration sites is characterized by FISH and other methods. In addition, it is possible that integration of a large LacO array in the genome may alter local chromatin conformation. Altogether, the versatility of the systems and methods disclosed herein offers a major technological advantage over conventional methods to study cellular organization.

The overall ease of targeting a new locus of polynucleotides with the systems and methods disclosed herein can facilitate broader studies of the relationship between perturbations in 3D polynucleotide organization and changes in cellular phenotypes. For example, different sgRNA design strategies can be used to target repetitive and non-repetitive genomic loci. Repetitive genomic loci can be easily targeted using a single sgRNA that has multiple targets within a defined genomic region. The human genome has abundant repetitive or repeat-derived sequences, many of which likely have important genome-organization roles. These repetitive sequences are candidates for large-scale screening experiments, opening the door to more high-throughput approaches to study the relationship between genome organization relative to nuclear compartments and cellular phenotype. In addition, non-repetitive genomic loci can be targeted using multiple sgRNAs or using a single sgRNA. To target a non-repetitive locus, a pool of tiling sgRNAs can be used as a starting point.

The provided systems and methods can also be useful for studying real-time dynamics of polynucleotide repositioning and the association and dissociation of cellular compartments from specific regions in living cells. In the CRISPR-GO system, genomic loci are targeted to the desired compartments via chemically induced physical interactions between dCas9-bound genomic loci and compartment-specific proteins. The inducible and reversible feature of CRISPR-GO prevents potential adverse effects from continuously repositioning chromatin DNA to a given nuclear compartment.

As one example, through the combined use of CRISPR-Cas9 live-cell genomic imaging and CRISPR-GO, relocalization of endogenous genomic loci to the nuclear periphery has been shown to occur in both a mitosis-dependent and -independent manner. During mitosis, the nuclear membrane breaks down in prometaphase and then reforms in telophase. The dramatic changes in chromatin and nuclear structure during mitosis could facilitate interactions between genomic loci and the nuclear membrane to create nuclear envelope tethering. During interphase, though chromatin structure remains relatively stable, a genomic locus can still form interactions with the nuclear periphery when it is in close proximity. Nuclear periphery tethering during interphase may rely on proximity between the targeted loci and nuclear periphery, and a genomic locus that is located distal to the nuclear periphery may less likely be tethered through the mitosis-independent manner.

The chemical induction process of some provided embodiments also allows for the investigation of the real-time association between a target polynucleotide locus and cellular compartments in living cells. For example, compared to the relatively slower repositioning to the nuclear periphery (within hours), colocalization between a genomic locus and Cajal bodies occurs at a much faster rate (within minutes), likely because Cajal body components are more diffuse throughout the nucleus. Using the disclosed systems and methods, it has been observed that colocalization between CBs and the target genomic loci could occur in two ways: one is rapid formation of de novo Cajal bodies at the genomic loci, and the other is re-localization of existing CBs with the target genomic loci, a phenomenon which has not been reported before. Previous work has suggested that Cajal bodies are formed by phase separation. The recruiting of nuclear body components (e.g., Coilin for CBs) by CRISPR-GO to targeted genomic loci may generate synthetic phase separation at the target chromatin loci.

The provided methods and systems have also been used to observe repression of an adjacent fluorescent reporter gene when repositioning a genomic locus to the nuclear periphery. Previous work reported different effects on gene expression after tethering LacO loci to the nuclear periphery. In particular, earlier studies have observed no change in transcription after LacO repeats were recruited to the nuclear periphery by LacI-Lamin B, and have shown that tethering LacO repeats to nuclear periphery by LacI-Emerin caused repression of adjacent genes. The systems disclosed herein have shown that repositioning the reporter gene to Emerin causes gene repression (˜59%).

The systems and methods disclosed herein have also been used to repress both adjacent reporter and endogenous genes after CRISPR-GO-mediated colocalization of a chromatin locus to CBs. Importantly, targeted colocalization of Cajal bodies with endogenous loci represses adjacent gene expression across long distances (>30 kb). This observed gene repression after targeting a genomic locus to CBs has not yet been reported. In contrast, the CRISPRi/a methods function by recruiting transcriptional effectors that mostly affect expression of local genes within a few kilobases around the target site. Thus, the provided methods and systems provide an important new method for regulating polynucleotide expression over a long distance. The methods and systems also provide the ability to control repositioning of target polynucleotides to diverse cellular compartments in a systematic way to investigate cellular effects and program polynucleotide regulation.

In some embodiments, the CRISPR-GO system can be programmed to recruit regulator proteins (e.g., activating or repressive effectors) for gene (e.g., target polynucleotide) expression regulation. Non-limiting examples of regulator proteins include heterochromatin protein 1 (e.g., HP1α, HP1β, and/or HP1γ), Krüppel-associated box-zinc finger protein (KRAB-ZFP), KRAB-associated protein 1 (KAP1), nucleosome remodeling deacetylase complex (NuRD), SET domain bifurcated 1 (SETDB1), DNA methyltransferase (e.g., DNMT3A, DNMT3L, DNMT3B), histone deacetylase (HDAC), SUV39H1, G9a, Ezh1/2, EED, Suz12, JARID2, AEBP2, RbAp48, PCL1, RBBP7/4, C17orf96, C10orf12, a truncated form thereof, a fragment thereof, and a combination thereof.

In some embodiments, the CRISPR-GO system can be programmed to alter cellular function, cell fate, cell growth, apoptosis, and/or cell differentiation, which can be achieved by repositioning developmental regulatory genomic regions and RNAs to different cellular compartments. This serves as an alternative way to using media-based approaches for inducing cell fate changes or using transcription factor cocktails to change cell fates. As described in Example 12, targeting telomeres to the nuclear periphery leads to a decrease in cell viability, causing a systematic change in gene expression levels including apoptosis genes, differentiation genes, and cell function genes, whereas targeting telomeres to Cajal bodies leads to an increase in cell viability that accompanies gene expression changes such as upregulation of growth genes and cell function genes.

In the cytoplasm of most asymmetric cells, mRNAs are transported along microtubules and actin filaments using motor proteins such as kinesins, dyneins, and myosins as compartment-specific proteins. In some embodiments, the CRISPR-GO system can be programmed for repositioning mRNAs along the cytoskeleton using these motor proteins. As described in Example 13, mRNAs can be repositioned to the plus ends of microtubules (MT+) using a motor protein such as kinesin-1 heavy chain (KIFSB), e.g., without the cargo binding tail domain, or mRNAs can be repositioned to the minus ends of microtubules (MT−) using a motor protein such as Bicaudal D2 (e.g., N-terminal fragment), which induces dynein-mediated cargo transport, or mRNAs can be repositioned along actin filaments (AF) using a motor protein such as myosin 5a (MYO5A).

In some embodiments, the CRISPR-GO system can be programmed to form nuclear compartments such as nuclear bodies that facilitate DNA repair (e.g., promote the formation of a complex to repair DNA double-strand breaks (DSB)) and lead to improved gene editing outcomes (e.g., enhanced homology-directed repair (HDR)). Non-limiting examples of compartment-specific proteins that can facilitate the formation of nuclear bodies include DNA repair genes such as 53BP1, Rad51, Rad52, Ubc9, UBL1, BLM, c-Abl, BCR/Abl, BRCA1/2, PALB2, RPA, Rad51AP1, Chk1, Arg, Hop2, Mndl, DMC1, or a combination thereof. In certain instances, oligomerizing 53BP1 (truncated, e.g., amino acids 1207-1711, or full-length) can be used to promote the formation of a complex to repair DNA double-strand breaks (DSB). In certain instances, Rad51, Rad52, Ubc9, UBL1, BLM, c-Abl, BCR/Abl, BRCA1/2, PALB2, RPA, Rad51AP1, Chk1, Arg, Hop2, Mndl, and/or DMC1 can be used to enhance homology-directed repair (HDR). As described in Example 14, 53BP1 foci formation is observed upon gene editing that could facilitate DSB resolution and DNA repair after CRISPR-mediated gene editing.

In some embodiments, the systems and methods disclosed herein are used with endogenous or synthetic oligomerizing proteins that self-aggregate to form an artificial protein/RNA/DNA aggregate, which can possess one or more unique chemical, physical, or biological properties (such as selective diffusion of specific proteins, RNA, or DNA; association or disassociation with other molecules; promotion or inhibition of gene regulation machineries; or promotion or inhibition of DNA recombination or stability machineries). Such an aggregate is referred to herein as a synthetic cellular phases (SCP). These aggregates can have strong effects on gene regulation. In some embodiments, a protein, protein domain, RNA, RNA domain, or combination thereof is coupled to a provided system to specifically form a desired SCP around desired chromatin DNA or RNA. In some embodiments, the provided system is useful for manipulating the spatiotemporal organization of genomic DNA and RNA components in the nucleus and/or cytoplasm and for regulating diverse cellular functions.

In some embodiments, the systems and methods comprise an inducible dimerization, wherein the dimerization is a chemically induced dimerization, light (e.g., optogenetically or chemo-optogenetically) induced dimerization, or an enzyme-catalyzed protein ligation. The dimerization can comprise homodimerization of identical dimerization domains or heterodimerization of two different dimerization domains.

In certain embodiments, the dimerization is a chemically induced dimerization mediated by a molecular ligand, such as a chemical inducer. In certain aspects, the dimerization system is selected from an ABA induced ABI/PYL1 dimerization system, a gibberellin (GA) induced GID1/GAI dimerization system, a rapamycin induced FRB/FKBP dimerization system, a TMP-HTag induced HaloTag/DHFR dimerization system, an FK1012 induced FKBP/FKBP dimerization system, an FK506 induced FKBP/Calcineurin A (CNA) dimerization system, an FKCsA induced FKBP/CyP-Fas dimerization system, a coumermycin induced GyrB/GyrB dimerization system, an HaXS induced SnapTag/HaloTag dimerization system, and an ABT-737 induced BCL-xL/Fab (AZ1) dimerization system. Other chemically induced dimerization systems are also contemplated.

In certain embodiments, the dimerization is light induced dimerization. Non-limiting examples of light induced dimerization include optogenetic and chemo-optogenetic dimerization systems. Optogenetic dimerization systems typically employ photosensitive proteins that undergo a conformational change upon illumination, and consequently, induce protein interaction. Chemo-optogenetic dimerization systems typically use photoactivatable and/or cleavable small molecule dimerizers, so that proximity can be induced and/or disrupted by light. See, e.g., Klewer et al., “Light-Induced Dimerization Approaches to Control Cellular Processes,” Chem. Eur. J. (2019) 25:1-13. Other light induced dimerization systems are also contemplated.

In certain embodiments, the dimerization is achieved using an enzyme-catalyzed reaction such as, e.g., enzyme-catalyzed protein ligation. As a non-limiting example, dimerization can be mediated by ligation of the dimerization domains catalyzed by a peptide ligase such as subtiligase or variants thereof. See, e.g., Henager, S., “Enzyme-catalyzed expressed protein ligation,” Nat Methods (2016) 13(11):925-927. Other dimerization systems using enzyme-catalyzed reactions are also contemplated.

In some embodiments, the targeted polynucleotide of the provided systems and methods comprises DNA, e.g., genomic DNA. In some embodiments, the target polynucleotide comprises RNA, e.g., mRNA, microRNA, siRNA, or non-coding RNA. Actuator moieties and related targeting systems suitable for use with the provided systems and methods include, for example, CRISPR-Cas (including all types of CRISPR, type I, II, III, IV, V, VI, e.g., Cas9, Cas12, Cas13,); Argonaute-mediated targeting or zinc finger targeting; TALE (transcription activator-like effectors); LacO-LacI or TetO-TetR; and specific pairs of DNA interacting protein or RNA domains. Cas9 and Cas13 can also target RNA in a sequence-dependent way, and can be used in this way with the provided system to re-localize RNA molecules to different cellular compartments. Cas proteins can lack DNA cleavage activity. The targeting systems can include sequence-specific guide RNAs or guide DNAs.

The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease, a derivative thereof, a variant thereof, or a fragment thereof. The actuator moiety can regulate expression or activity of a gene and/or edit the sequence of a nucleic acid (e.g., a gene and/or gene product). In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity. An actuator moiety having reduced or minimal nuclease activity can regulate expression and/or activity of a gene by physical obstruction of a target polynucleotide or recruitment of additional factors effective to suppress or enhance expression of the target polynucleotide. In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. In some embodiments, the actuator moiety is a DNA-guided actuator moiety. In some embodiments, the actuator moiety is an RNA-guided actuator moiety. An actuator moiety can regulate expression or activity of a gene and/or edit a nucleic acid sequence, whether exogenous or endogenous.

Any suitable nuclease can be used in an actuator moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA-binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof any variant thereof and any fragment thereof.

In some embodiments, the actuator moiety comprises a CRISPR-associated (Cas) protein or a Cas nuclease which functions in a non-naturally occurring CRISPR (Clustered Regularly Interspaced Short Palindromic Repeats)/Cas (CRISPR-associated) system. In bacteria, this system can provide adaptive immunity against foreign DNA (Barrangou, R., et al, “CRISPR provides acquired resistance against viruses in prokaryotes,” Science (2007) 315: 1709-1712; Makarova, K. S., et al, “Evolution and classification of the CRISPR-Cas systems,” Nat Rev Microbiol (2011) 9:467-477; Garneau, J. E., et al, “The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39: 9275-9282).

In a wide variety of organisms including diverse mammals, animals, plants, and yeast, a CRISPR/Cas system (e.g., modified and/or unmodified) can be utilized as a genome engineering tool. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E2 86; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62; Deltcheva, E., et al, “CRISPR RNA maturation by trans-encoded small RNA and host factor RNase III,” Nature (201 1) 471:602-607), and has been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31: 233-239; Sander, J. D. & Joung, J. K, “CRISPR-Cas systems for editing, regulating and targeting genomes,” Nature Biotechnol. (2014) 32:347-355).

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity. An actuator moiety comprising a Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products.

In some embodiments, the actuator moiety comprises a Cas protein that forms a complex with a guide nucleic acid, such as a guide RNA. In some embodiments, the actuator moiety comprises a Cas protein that forms a complex with a single guide nucleic acid, such as a single guide RNA (sgRNA). In some embodiments, the actuator moiety comprises a RNA-binding protein (RBP) optionally complexed with a guide nucleic acid, such as a guide RNA (e.g., sgRNA), which is able to form a complex with a Cas protein. In some embodiments, the actuator moiety comprises a nuclease-null DNA-binding protein derived from a DNA nuclease that can induce transcriptional activation or repression of a target DNA sequence. In some embodiments, the actuator moiety comprises a nuclease-null RNA-binding protein derived from an RNA nuclease that can induce transcriptional activation or repression of a target RNA sequence.

Any suitable CRISPR/Cas system can be used. A CRISPR/Cas system can be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

An actuator moiety comprising a Cas protein can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas10, Cas1Od, Cas10, Cas1Od, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wild-type or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like nuclease domain. The RuvC and HNH domains can each cut a different strand of double-stranded DNA to make a double-stranded break in the DNA. A Cas protein can comprise only one nuclease domain (e.g., Cpf1 comprises RuvC domain but lacks HNH domain).

A Cas protein can comprise an amino acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

A Cas protein can be a fusion protein. For example, a Cas protein can be fused to a cleavage domain, an epigenetic modification domain, a transcriptional activation domain, or a transcriptional repressor domain. A Cas protein can also be fused to a heterologous polypeptide providing increased or decreased stability. The fused domain or heterologous polypeptide can be located at the N-terminus, the C-terminus, or internally within the Cas protein.

A Cas protein can be provided in any form. For example, a Cas protein can be provided in the form of a protein, such as a Cas protein alone or complexed with a guide nucleic acid. A Cas protein can be provided in the form of a nucleic acid encoding the Cas protein, such as an RNA (e.g., messenger RNA (mRNA)) or DNA. The nucleic acid encoding the Cas protein can be codon optimized for efficient translation into protein in a particular cell or organism.

Nucleic acids encoding Cas proteins can be stably integrated in the genome of the cell. Nucleic acids encoding Cas proteins can be operably linked to a promoter active in the cell. Nucleic acids encoding Cas proteins can be operably linked to a promoter in an expression construct. Expression constructs can include any nucleic acid constructs capable of directing expression of a gene or other nucleic acid sequence of interest (e.g., a Cas gene) and which can transfer such a nucleic acid sequence of interest to a target cell.

In some embodiments, a Cas protein is a dead Cas protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

A dCas9 polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell. The nucleic acids targeted by the guide RNA can be any that are expressed in a cell such as an immune cell. The nucleic acids targeted may be a gene involved in immune cell regulation. In some embodiments, the nucleic acid is associated with cancer. The nucleic acid associated with cancer can be a cell cycle gene, cell response gene, apoptosis gene, or phagocytosis gene. The recombinant guide RNA can be recognized by a CRISPR protein, a nuclease-null CRISPR protein, variants thereof, or derivatives thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type Cas9 activity).

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A dead Cas protein can comprise one or more mutations relative to a wild-type version of the protein. The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

As non-limiting examples, residues D10, G12, G17, E762, H840, N854, N863, H982, H983, A984, D986, and/or A987 (or the corresponding mutations of any of the Cas proteins) can be mutated. For example, e.g., D10A, G12A, G17A, E762A, H840A, N854A, N863A, H982A, H983A, A984A, and/or D986A. Mutations other than alanine substitutions can be suitable.

A D10A mutation can be combined with one or more of H840A, N854A, or N856A mutations to produce a Cas9 protein substantially lacking DNA cleavage activity (e.g., a dead Cas9 protein). A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity. In some embodiments, the Cas9 protein is a Cas9 protein from S. pyogenes (e.g., SwissProt accession number Q99ZW2). In some embodiments, the Cas9 protein is a Cas9 from S.aureus (e.g., SwissProt accession number J7RUA5). In some embodiments, the Cas9 protein is a modified version of a Cas9 protein from S. pyogenes or S. aureus. In some embodiments, the Cas9 protein is derived from a Cas9 protein from S. pyogenes or S. aureus. For example, a S. pyogenes or S. aureus Cas9 protein lacking cleavage activity.

Cas9 can generally refer to a polypeptide with at least about 5%, 10%, 20%, 30%, 40%, 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most about 5%, 10%, 20%, 30%, 40%, 50%, 60%, 70%, 80%, 90%, 100% sequence identity and/or sequence similarity to a wild type exemplary Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

In some embodiments, an actuator moiety comprises an RNA-binding protein complexed with a guide RNA that hybridizes to a target polynucleotide. Non-limiting examples of RNA-binding proteins include ADAR1 or ADAR2 and non-limiting examples of guide RNA include ADAR-recruiting RNAs (arRNAs) (Qu, L., et al, “Programmable RNA editing by recruiting endogenous ADAR using engineered RNAs,” Nat Biotechnol. (2019) Jul. 15. doi: 10.1038/s41587-019-0178-z).

In some embodiments, an actuator moiety comprises a “zinc finger nuclease” or “ZFN.” ZFNs refer to a fusion between a cleavage domain, such as a cleavage domain of FokI, and at least one zinc finger motif (e.g., at least 2, 3, 4, or 5 zinc finger motifs) which can bind polynucleotides such as DNA and RNA. The heterodimerization at certain positions in a polynucleotide of two individual ZFNs in certain orientation and spacing can lead to cleavage of the polynucleotide. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain. Exemplary ZFNs include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

In some embodiments, an actuator moiety comprising a ZFN can generate a double-strand break in a target polynucleotide, such as DNA. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template that contains homology arms flanking sites of the target DNA can be provided. In some embodiments, a ZFN is a zinc finger nickase which induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33. In some embodiments, a ZFN binds a polynucleotide (e.g., DNA and/or RNA) but is unable to cleave the polynucleotide.

In some embodiments, the cleavage domain of an actuator moiety comprising a ZFN comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type cleavage domain. The modified form of the cleavage domain can have no substantial nucleic acid-cleaving activity. In some embodiments, the cleavage domain is enzymatically inactive.

In some embodiments, an actuator moiety comprises a “TALEN” or “TAL-effector nuclease.” TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR. In some cases, a single-stranded donor DNA repair template is provided to promote HDR. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type nuclease domain. The modified form of the nuclease domain can have no substantial nucleic acid-cleaving activity. In some embodiments, the nuclease domain is enzymatically inactive.

In some embodiments, the transcription activator-like effector (TALE) protein is fused to a domain that can modulate transcription and does not comprise a nuclease. In some embodiments, the transcription activator-like effector (TALE) protein is designed to function as a transcriptional activator. In some embodiments, the transcription activator-like effector (TALE) protein is designed to function as a transcriptional repressor. For example, the DNA-binding domain of the transcription activator-like effector (TALE) protein can be fused (e.g., linked) to one or more transcriptional activation domains, or to one or more transcriptional repression domains. Non-limiting examples of a transcriptional activation domain include a herpes simplex VP16 activation domain and a tetrameric repeat of the VP16 activation domain, e.g., a VP64 activation domain. A non-limiting example of a transcriptional repression domain includes a Krüppel-associated box domain.

In some embodiments, an actuator moiety comprises a meganuclease. Meganucleases generally refer to rare-cutting endonucleases or homing endonucleases that can be highly specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-MsoI meganuclease, I-SceI meganuclease, variants thereof, derivatives thereof, and fragments thereof. Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

In some embodiments, the nuclease domain of a meganuclease comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA.

In some embodiments, the actuator moiety is fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the enzyme. In other cases, the actuator moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-κB p65 subunit, Epstein-Barr virus R transactivator (Rta) and are described in Chavez et al., Nat Methods, 2015, 12(4):326-328 and U.S. Patent App. Publ. No. 20140068797. Non-limiting exemplary repression domains include the KRAB (Krüppel-associated box) domain of Kox1, the Mad mSIN3 interaction domain (SID), ERF repressor domain (ERD), and are described in Chavez et al., Nat Methods, 2015, 12(4):326-328 and U.S. Patent App. Publ. No. 20140068797. An actuator moiety can also be fused to a heterologous polypeptide providing increased or decreased stability. The fused domain or heterologous polypeptide can be located at the N-terminus, the C-terminus, or internally within the actuator moiety.

An actuator moiety can comprise a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g. eBFP, eBFP2, Azurite, mKalamal, GFPuv, Sapphire, T-sapphire), cyan fluorescent proteins (e.g. eCFP, Cerulean, CyPet, AmCyanl1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

Any suitable delivery method can be used for introducing the systems of the disclosure comprising polypeptides and/or nucleic acid encoding the polypeptides into a cell. The system components (e.g., compartment-specific protein linked to a first dimerization domain, actuator moiety linked to a second dimerization domain) can or be delivered simultaneously or temporally separated. The choice of method of genetic modification can be dependent on the type of cell being transformed and/or the circumstances under which the transformation is taking place (e.g., in vitro, ex vivo, or in vivo).

A method of delivery can involve introducing into a cell (or a population of cells) one or more polynucleotides comprising nucleic acid sequences encoding the system components of the disclosure (e.g., compartment-specific protein linked to a first dimerization domain, actuator moiety linked to a second dimerization domain). Suitable polynucleotides comprising nucleic acid sequences encoding the system components of the disclosure can include expression vectors, wherein an expression vector comprising a nucleic acid sequence encoding one or more system components of the disclosure (e.g., compartment-specific protein linked to a first dimerization domain, actuator moiety linked to a second dimerization domain) is a recombinant expression vector.

Non-limiting examples of delivery methods or transformation include viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, use of cell permeable peptides, and nanoparticle-mediated nucleic acid delivery.

In some embodiments, the present disclosure provides methods comprising delivering one or more polynucleotides, oligonucleotides, or vectors as described herein, or one or more transcripts thereof, and/or one or proteins transcribed therefrom, to a cell. In some embodiments, the disclosure further provides cells produced by such methods, and organisms (such as animals, plants, or fungi) comprising or produced from such cells. In certain embodiments, the cells produced by such methods comprise polynucleotides (e.g., vectors) that encode a compartment-specific protein linked to a first dimerization domain and actuator moiety linked to a second dimerization domain.

Any suitable vector compatible with the cell can be used with the methods of the disclosure. Non-limiting examples of vectors for eukaryotic cells include pXT1, pSG5 (Stratagene™), pSVK3, pBPV, pMSG, and pSVLSV40 (Pharmacia™).

In some embodiments, a polynucleotide sequence encoding a system component (e.g., compartment-specific protein linked to a first dimerization domain, actuator moiety linked to a second dimerization domain) is operably linked to a control element, e.g., a transcriptional control element, such as a promoter. The transcriptional control element can be functional in either a eukaryotic cell, e.g., a mammalian cell, or a prokaryotic cell (e.g., bacterial or archaeal cell). In some embodiments, a polynucleotide sequence encoding a system component is operably linked to multiple control elements that allow expression of the polynucleotide sequence in prokaryotic and/or eukaryotic cells.

Promoters that can be used with the systems and methods of the disclosure include, for example, promoters active in a eukaryotic, mammalian, non-human mammalian, or human cells. The promoter can be an inducible or constitutively active promoter. Alternatively or additionally, the promoter can be tissue- or cell-specific.

Non-limiting examples of suitable eukaryotic promoters (i.e., promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression.

In some embodiments, the target polynucleotide is positioned by the provided systems and methods in an inner nuclear membrane. Compartment-specific proteins suitable for targeting the inner nuclear membrane include, but are not limited to, Emerin, Lap2beta, and Lamin B.

In some embodiments, the target polynucleotide is positioned by the provided systems and methods in a Cajal body. Compartment-specific proteins suitable for targeting Cajal bodies include, but are not limited to, Coilin, SMN, Gemin 3, SmD1, and SmE.

In some embodiments, the target polynucleotide is positioned by the provided systems and methods in nuclear speckles. Compartment-specific proteins suitable for targeting nuclear speckles include, but are not limited to, SC35.

In some embodiments, the target polynucleotide is positioned by the provided systems and methods in a PML body. Compartment-specific proteins suitable for targeting PML bodies include, but are not limited to, PML and SP100.

In some embodiments, the target polynucleotide is positioned by the provided systems and methods in a nuclear pore complex. Compartment-specific proteins suitable for targeting nuclear pore complexes include, but are not limited to, Nup50, Nup98, Nup53, Nup153, and Nup62.

In some embodiments, the target polynucleotide is positioned by the provided systems and methods in a nucleolus. Compartment-specific proteins suitable for targeting the nucleolus include, but are not limited to, nuclear protein B23.

In some embodiments, the target polynucleotide is positioned by the provided systems and methods in a P granule. Compartment-specific proteins suitable for targeting P granules include, but are not limited to, RGG domain proteins (e.g., PGL-1 and PGL-3), Dead box proteins, and GLH-1-4.

In some embodiments, the target polynucleotide is positioned by the provided systems and methods in a GW body. Compartment-specific proteins suitable for targeting GW bodies include, but are not limited to, GW182.

In some embodiments, the target polynucleotide is positioned by the provided systems and methods in a stress granule. Compartment-specific proteins suitable for targeting stress granules include, but are not limited to, G3BP (Ras-GAP SH3 binding proteins), TIA-1 (T-cell intracellular antigen), eIF2, and eIF4E.

In some embodiments, the target polynucleotide is positioned by the provided systems and methods in a sponge body. Compartment-specific proteins suitable for targeting sponge bodies include, but are not limited to, EXu, Btz, Tral, Cup, eIF4E, Me31B, Yps, Gus, Dcp1/2, Sqd, BicC, Hrb27C, and Bru.

In some embodiments, the target polynucleotide is positioned by the provided systems and methods in a cytoplasmic prion protein induced ribonucleoprotein (CyPrP-RNP) granule. Compartment-specific proteins suitable for targeting CyPrP-RNP granules include, but are not limited to, Dcp1a, DDX6/Rck/p54/Me31B/Dhh1, and Dicer.

In some embodiments, the target polynucleotide is positioned by the provided systems and methods in a U body. Compartment-specific proteins suitable for targeting U bodies include, but are not limited to, one or more uridine-rich small nuclear ribonucleoproteins U1, U2, U4/U6 and U5; LSm1-7; and the survival of motor neurons (SMN) protein.

In some embodiments, the target polynucleotide is positioned by the provided systems and methods in the endoplasmic reticulum. Compartment-specific proteins suitable for targeting the endoplasmic reticulum include, but are not limited to, Calreticulin, Calnexin, PDI, GRP 78, and GRP 94.

In some embodiments, the target polynucleotide is positioned by the provided systems and methods in a mitochondrium. Compartment-specific proteins suitable for targeting mitochondria include, but are not limited to, HIF1A, PLN, Cox1, Hexokinase, and TOMM40.

In some embodiments, the target polynucleotide is positioned by the provided systems and methods in the plasma membrane. Compartment-specific proteins suitable for targeting the plasma membrane include, but are not limited to, sodium potassium ATPase, CD98, Cadherins, and plasma membrane calcium ATPase (PMCA).

In some embodiments, the target polynucleotide is positioned by the provided systems and methods in golgi. Compartment-specific proteins suitable for targeting golgi include, but are not limited to, GM130, MAN2A1, MAN2A2, GLG1, B4GALT1, RCAS1, and GRASP65.

In some embodiments, the target polynucleotide is positioned by the provided systems and methods in a ribosome. Compartment-specific proteins suitable for targeting ribosomes include, but are not limited to, AGO2, MTOR, PTEN, RPL26, FBL, and RPS3.

In some embodiments, the target polynucleotide is positioned by the provided systems and methods in a proteasome. Compartment-specific proteins suitable for targeting proteasomes include, but are not limited to, PSMA1, PSMB5, PSMC1, PSMD1, and PSMD7.

In some embodiments, the target polynucleotide is positioned by the provided systems and methods in an endosome. Compartment-specific proteins suitable for targeting endosomes include, but are not limited to, CFTR, ADRB1, EGFR, IGF2R, AP2S1, CD4, HLA-A, Coveolin, RABS, and ErbB2.

In some embodiments, the target polynucleotide is positioned by the provided systems and methods in a liposome. Compartment-specific proteins suitable for targeting liposomes include, but are not limited to, EEA1, LAMTOR2, and LAMTOR4.

Other cell compartments that can be targeted with the systems and methods disclosed herein include RNP bodies, mitotic spindles, histone locus bodies, heterochromatin regions, and the cytoskeleton. Additional compartments are also contemplated.

The target polynucleotide can be endogenous or exogenous to the cell compartment to which it is positioned. The target polynucleotide can be endogenous or exogenous to the cell. The target polynucleotide can be human or non-human. The target polynucleotide can be virally derived, a plasmids, a ribonucleoprotein, or a synthesized RNA or DNA strand.

The methods and systems disclosed herein are suitable for use in multiplexed processes in which multiple polynucleotides are repositioned to the same or different cellular compartments.

In some embodiments, the provided systems and methods are used to mediate de novo cellular compartment (e.g., nuclear body) formation at targeted polynucleotide (e.g., genomic) loci, providing a potential method to initiate membraneless organelle formation via liquid-liquid phase separation. Membraneless compartmentalization of the subcellular space occurs by liquid-liquid phase separation. Heterotypic cooperative weak interactions enable rapid rearrangements within liquid compartments. Intrinsically disordered proteins play important roles in phase transitions due to their structural plasticity and prion-like properties. Cells dynamically control the extent and duration of phase transitions. Molecular seeds such as DNA, RNA or poly(ADP-ribose) (PAR) can trigger phase transitions in a stimulus- and context-specific manner. Chaperones, disintegrase machineries, and post-translational modifications cooperate to control phase transitions. A continuum of aggregation propensities exists and cells employ an unanticipated broad range of material states in proteinaceous assemblies. These can progress into pathological aggregates associated with neurodegenerative diseases.

Examples of synthetic phases that can be formed using the systems and methods disclosed herein include, but are not limited to, synthetic PML bodies that can have roles in viral defense and telomere maintenance, synthetic nuclear speckles and paraspeckles that can be stress inducible anti-apoptotic structures, synthetic gems that can be hubs for factors involved in neurodegeneration, synthetic architectural RNAs that can seed nuclear bodies, synthetic nucleoli, synthetic heterochromatin or euchromatin, synthetic histone locus bodies that can be sites of FLASH accumulation and enhance histone mRNA processing, synthetic chromatin packing systems that can involve the use of Xist to silence in cis the whole chromosome, synthetic epigenetic phases, synthetic (cytoplasmic) P bodies, synthetic stress bodies, synthetic germ granules that can generate sexual cells upon meiosis in the developing embryo, synthetic mRNP granules in neurodegenerative disease, synthetic posttranslational modifications (PTM) that can regulate membrane-less organelle structure and dynamics, synthetic IDP (intrinsically disordered proteins) forming aggregates, and synthetic prion like domains (PLDs) or RGG-rich low-complexity domains (LCD). Other non-endogenous protein/RNA aggregates to which polynucleotides can be positioned include β-amyloid bodies, mRNA aggregates, Xist packaging complexes, and others.

The controlled positioning of polynucleotides as described herein can be used to regulate, modify, or influence, for example, DNA interaction with RNA polymerases, transcription factors, pioneer factors, mediators, DNA looping molecules, and other DNA associated proteins; epigenetic modification marks or euchromatin/heterochromatin modulating enzymes (e.g., HP1); chromatin compactness and other biophysics/biochemical properties; gene editing, including recombination, NHEJ, or HDR; genome stability and cancer; DNA repair processes; and mRNA metabolism through splicing, degradation, translation, methylation, localization, and interaction with other chaperones and RNA-binding proteins.

The methods and systems disclosed herein can be used to establish inducible and reversible disease models to understand disease mechanism. For example, the provided systems and methods can be used to investigate diseases caused by protein/RNA misfolding or aggregations. Proteome imbalances are associated with aging and often involve abundant proteins that exceed solubility and tend to form intracellular and extracellular aggregates. Aging is a risk factor for the onset of several protein misfolding disorders (PMDs), particularly for progressive neurodegeneration. Protein aggregation is the primary hallmark of neurodegeneration, including amyloid beta (Ab) and tau aggregation in Alzheimer's disease (AD), intracellular alpha-synuclein aggregates in Parkinson's disease (PD) and multisystem atrophy, polyQ-driven protein aggregates in Huntington's disease (HD), PrPSc in prion diseases, and TDP-43 and FET protein aggregates in amyotrophic lateral sclerosis (ALS) and frontotemporal dementia (FTD), just to list a few examples. Although the chemical nature and the (patho)physiological topology of the proteins involved in plaque formation differ, the principles that govern their aggregation appear surprisingly similar, and the provided methods and systems can be used to position target polynucleotides at these plaques or aggregates.

The systems and methods disclosed herein can be used to control cell differentiation by repositioning key driver genes into different nuclear compartments. The systems and methods can be used to enhance antibody production by controlling the recombination rate at the endogenous VD(J) locus. The systems and methods can be used for mitigating Alzheimer's by eliminating the formation of misfolding protein bodies.

The systems and methods disclosed herein are broadly applicable in all kingdoms of life, including plants, bacteria, archaea, yeast, fishes, insects, birds, mammals, mice, pigs, and humans. The systems and methods can be used in living whole organisms or in tissue or cells.

### IV. Examples

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure.

### Example 1. Development of a Chemical-Inducible CRISPR-GO Platform for Target-Specific Genomic Repositioning

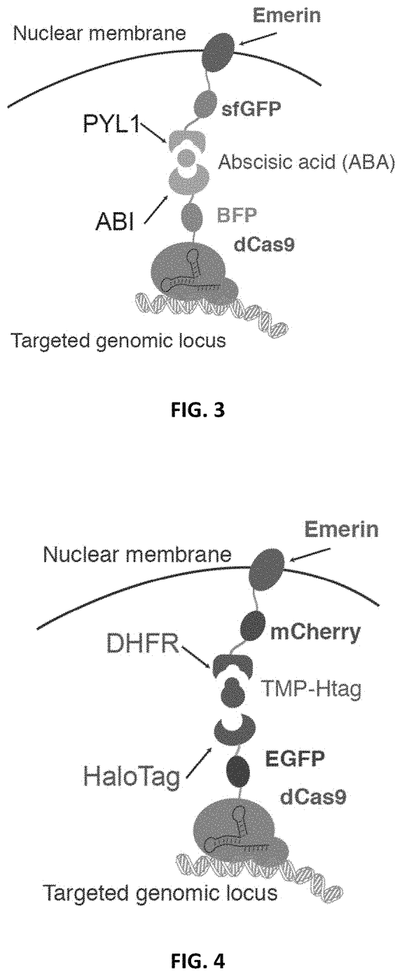

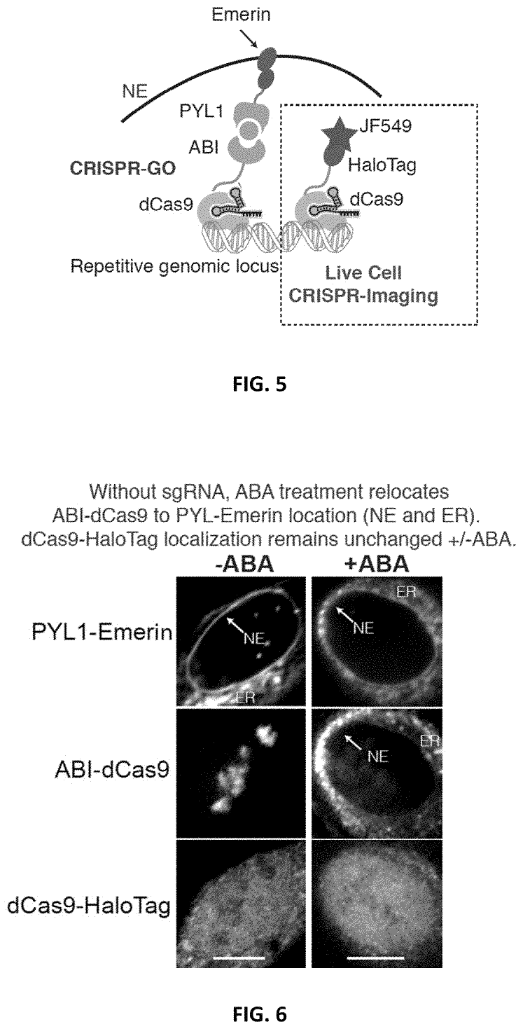

To implement an inducible CRISPR-mediated chromatin repositioning system, two chemical-inducible heterodimerization systems were tested. The first was an abscisic acid (ABA) inducible ABI/PYL1 system, and the second was a TMP-Htag (Trimethoprim-Haloligand) inducible DHFR/HaloTag system. For both systems, the Streptococcus pyogenes dCas9 (D10A & H840A) protein was fused to one heterodimer, and an inner nuclear envelope (NE) protein, Emerin, was fused to the cognate heterodimer (FIGS. 2-4). Emerin, encoded by the EMD gene, is among a group of LEM (LAP2, Emerin, MAN1)-domain proteins that mediate chromatin organization at the nuclear inner membrane. Emerin is synthesized in the cytoplasm, inserted into endoplasmic reticulum (ER), and then translocated to NE through diffusion within the contiguous ER/NE membranes (Berk et al., 2013). U2OS human bone osteosarcoma epithelial cell lines were created using lentiviral transduction that stably expressed each dimerization system. In these cell lines, addition of ABA caused spatial re-localization of ABI-BFP-dCas9 protein from within the nuclear interior to the NE and ER, due to its dimerization with PYL1-GFP-Emerin (FIGS. 5 and 6). In contrast, the TMP-Htag inducible system showed no evident effects on the co-localization of dCas9-EGFP-HaloTag and DHFR-mCherry-Emerin with the ligand. Therefore, the ABA-inducible ABI/PYL1 heterodimerization system was used for later experiments.

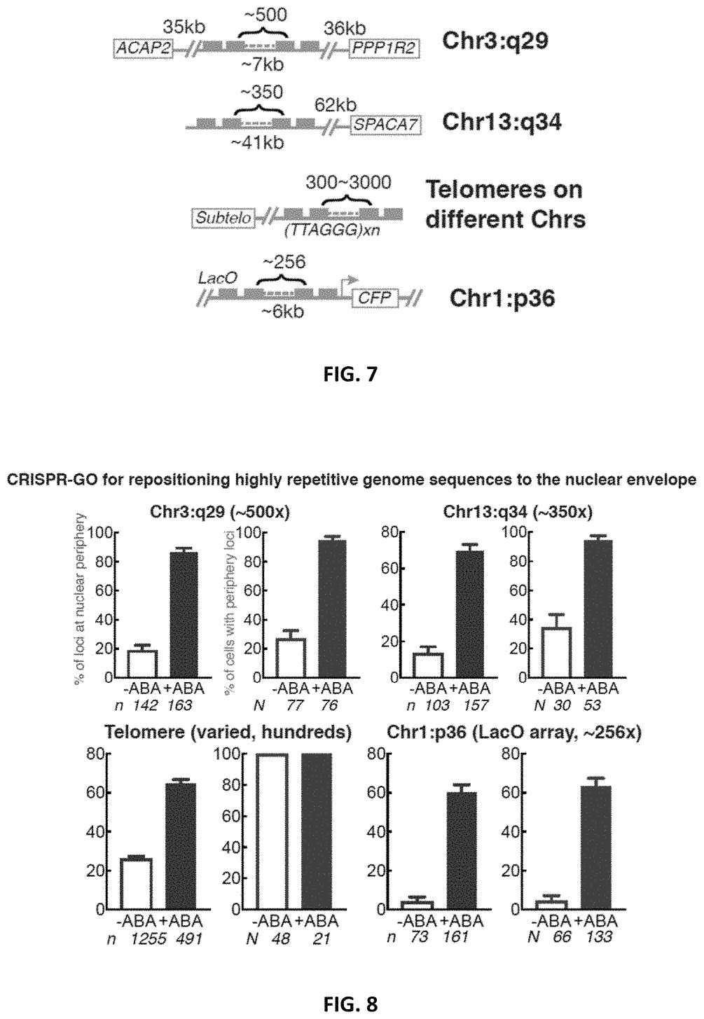

To test if the ABA-inducible CRISPR-GO system was able to alter the position of chromosomes, an endogenous locus on Chromosome 3 (Chr3) was targeted. An sgRNA targeting a highly repetitive (˜500×) region within Chromosome 3 (3q29) was lentivirally transduced into the U2OS cell line that stably expresses ABI-BFP-dCas9 and PYL1-GFP-Emerin (FIGS. 2 and 7). Given that AB1-BFP-dCas9 was mostly recruited to PYL-GFP-Emerin localization (NE and ER) after ABA treatment, another independent CRISPR-Cas9 imaging component, a dCas9-HaloTag fusion protein, was added to visualize the position of the targeted Chr3 genomic locus (FIGS. 5 and 6). In the presence of the sgChr3, the JF549-HaloTag dye was added to the culture medium to bind to dCas9-HaloTag and enable visualization of the targeted Chr3:q29 locus in living cells. The sgChr3 mediates both CRISPR-Cas9 imaging (via dCas9-HaloTag) and CRISPR-GO genomic re-localization (via AB1-dCas9) by targeting multiple repeats within the same Chr3:q29 genomic region. It was also confirmed that, in the absence of sgRNA, the dCas9-HaloTag localization was unaffected by the ABA-mediated heterodimerization between AB1-BFP-dCas9 and PYL-GFP-Emerin (FIG. 6).

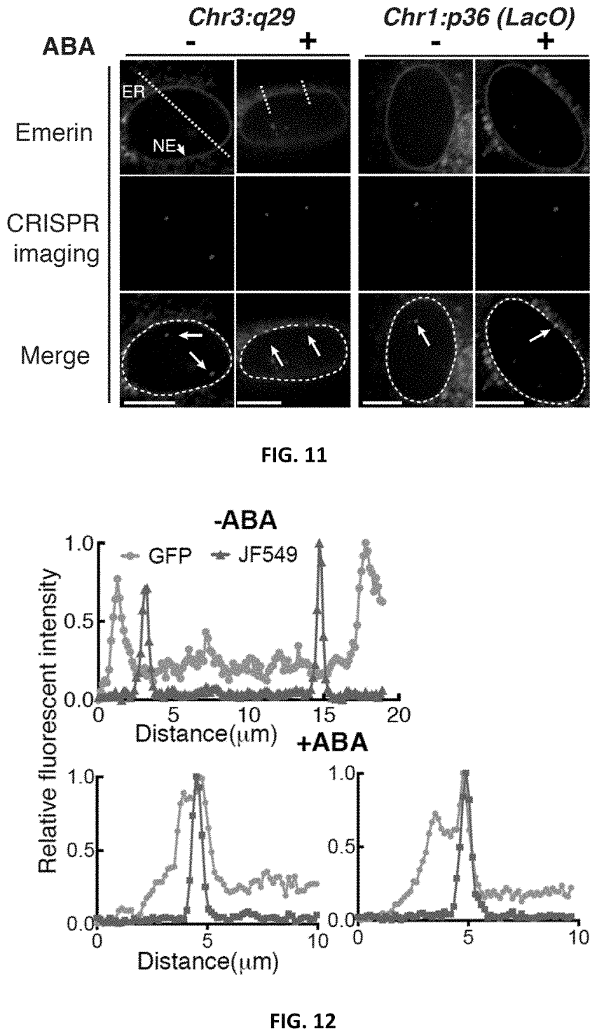

After 2 days of ABA treatment, significantly increased tethering of targeted Chr3 locus to the nuclear periphery was observed as compared to cells without ABA treatment (FIGS. 8 and 10-12). Without ABA treatment, 19% of dCas9-HaloTag labeled Chr3 loci were positioned at the nuclear periphery marked by PYL1-GFP-Emerin, while the majority of labeled loci remained within the nuclear interior (142 loci analyzed, FIG. 8). In contrast, 87% of labeled Chr3 loci repositioned to the nuclear periphery in ABA-treated cells (163 loci, FIG. 8). ABA treatment also increased the percentage of cells showing at least one Chr3 locus localized to the nuclear membrane from 27% (77 cells) to 95% (76 cells, FIG. 8). The significant increase of both repositioned genomic loci (p<0.0001) and cells (p<0.0001) with chemical treatment suggests that the systems disclosed herein are efficient in repositioning highly repetitive polynucleotides such as endogenous genomic loci in cells.

### Example 2. Efficient Repositioning of Repetitive Genomic Loci by the CRISPR-GO System

In addition to the Chr3:q29 locus, repositioning other highly repetitive endogenous genomic loci, including Chr13 locus and telomeres, to the nuclear periphery was tested. Using an sgRNA targeting repetitive region (˜350× repeats) on Chromosome 13q34 (Chr13) (FIG. 7), the percentage of tethered Chr13 loci increased from 13% (n=103) to 69% (n=157, p<0.0001), and the percentage of cells containing at least one periphery-localized locus increased from 34% (n=30) to 94% (n=53, FIG. 8, p<0.0001). Similarly, CRISPR-GO-containing cells with a telomere-targeting sgRNA were transduced to test whether telomeres could also be repositioned with our system. TRF1-mCherry, a telomere marker, was also co-expressed to visualize telomeres. In this case, the percentage of periphery-localized telomere loci increased from 26% (n=1255) to 65% (n=491, FIG. 8, p<0.0001).

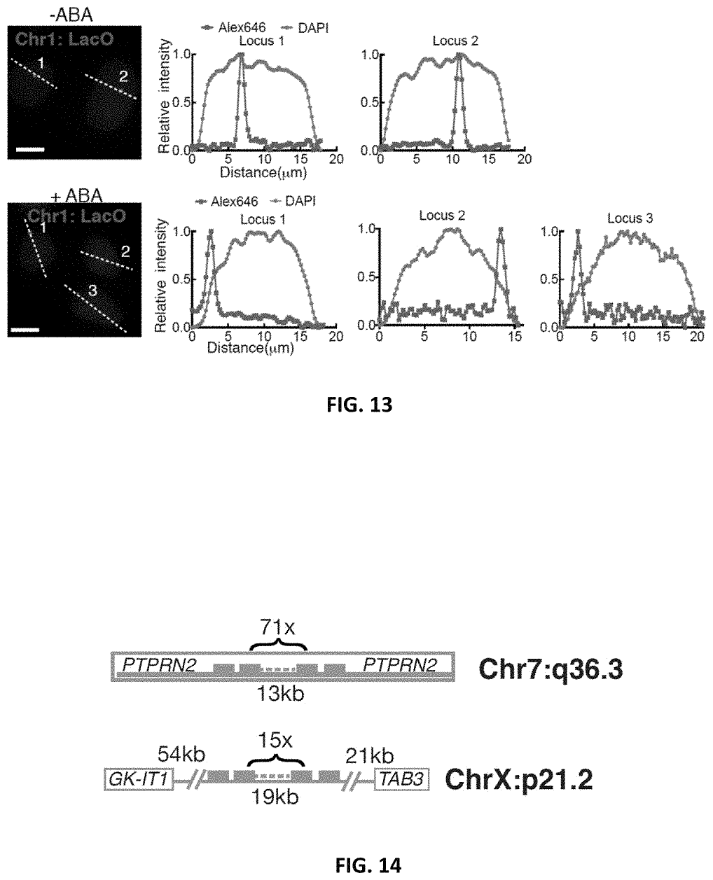

A synthetically integrated LacO array located at Chromosome 1p36 was also targeted in a U2OS 2-6-3 reporter cell line previously used for studying chromosome repositioning (FIG. 7). Using an sgRNA targeting the LacO sequence, CRISPR-Cas9 imaging in living cells revealed that the percentage of nuclear periphery-tethered targeted genomic loci increased from 4% (n=73) to 60% (n=161, p<0.0001), and the percentage of cells containing at least one periphery-localized locus increased from 4.5% (n=66) to 65% (n=133) (FIG. 8, 1G, p<0.0001). Fluorescent in situ hybridization (FISH) staining in fixed cells with DAPI further confirmed that the majority of LacO loci localized at the nuclear periphery after ABA treatment (FIG. 13).

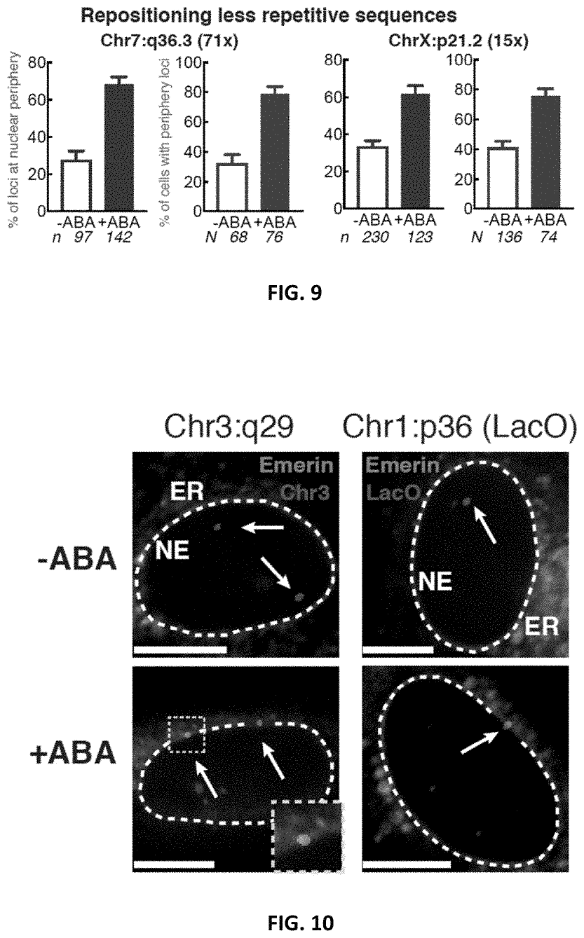

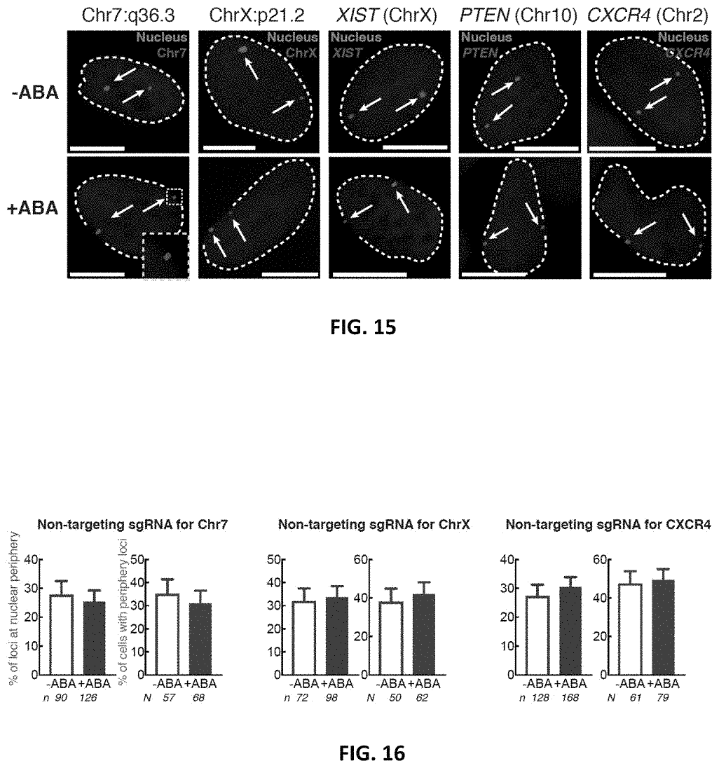

The efficiency of the CRISPR-GO system in repositioning less repetitive (<100 repeats) sequences was next tested. A genomic region on Chr7 q36.3 containing ˜71 sgRNA-targetable repeats and a genomic region on ChrX p21.2 containing ˜15 sgRNA-targetable repeats were chosen as targets (FIG. 14). We visualized The position of the targeted genomic loci was visualized using 3D-FISH and the nucleus was stained by DAPI (FIG. 15). After 3 days of ABA treatment, the percentage of periphery-localized loci increased from 28% (n=97) to 68% (n=142, p<0.0001) for Chr7 and from 33% (n=230) to 62% (n=123, p<0.0001) for ChrX. The percentage of cells containing at least one periphery-adjacent locus increased from 32% (n=68) to 79% (n=76, p<0.0001) for Chr7 and from 41% (n=136) to 76% (n=74, p<0.0001) for ChrX (FIG. 9). When using a non-targeting sgRNA as a control, the percentages of Chr7 and ChrX at the nuclear periphery were similar to those seen with non-ABA treated samples and remained unchanged after ABA treatment (FIG. 16). Together, these results suggest that the chemical inducible systems provided herein are efficient in repositioning highly repetitive and less repetitive sequences to cellular compartments such as the nuclear periphery.

### Example 3. Efficient Repositioning of Non-Repetitive Genomic Loci by the CRISPR-GO System

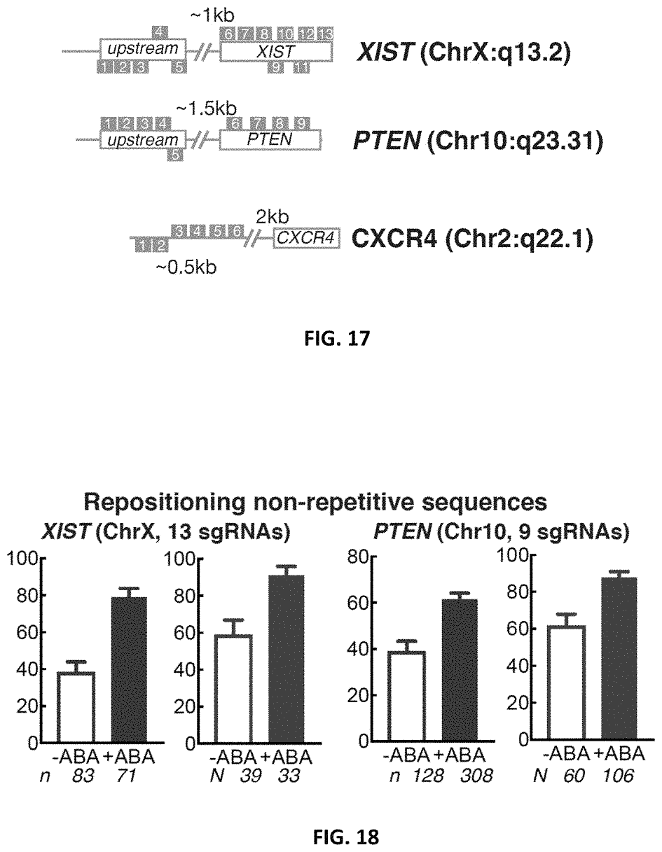

Though repetitive sequences are abundantly present in human genome, it is of further interest if the CRISPR-GO system enabled repositioning non-repetitive genomic loci. The non-repetitive gene XIST located at ChrX q13.2, was first targeted and 13 sgRNAs tiling the XIST genomic region were designed (FIG. 17). All constructs were lentivirally transduced into U2OS cells that stably expressed the CRISPR-GO system. With ABA treatment, the percentage of periphery-localized XIST loci increased from 39% (n=83) to 79% (n=71, p<0.0001), and the percentage of cells containing periphery-localized loci increased from 59% (n=39) to 90% (n=33) (FIG. 18, 1H, p=0.0028). Using a pool of 9 sgRNAs targeting regions adjacent to and within the gene PTEN at Chr10 (FIG. 17), the CRISPR-GO system increased the percentage of periphery localized PTEN loci from 39% (n=128) to 61% (n=308, p<0.0001) and the percentage of cells containing at least one periphery-adjacent locus increased from 62% (n=60) to 88% (n=106, p=0.0002) (FIGS. 15 and 18).

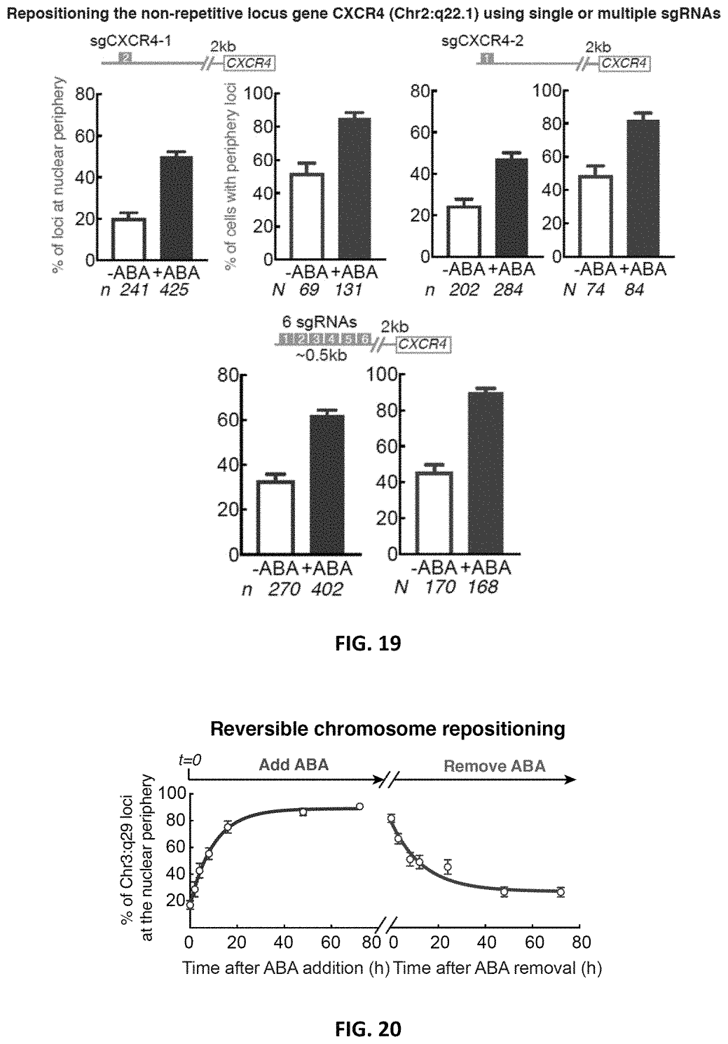

Whether a single sgRNA targeting a non-repetitive region is sufficient to re-reposition a genomic locus was also tested. Using a single sgRNA (sgCXCR4-1) targeting the CXCR4 locus at Chr2, the percentage of periphery localized CXCR4 loci increased from 20% (n=241) to 50% (n=425, p<0.0001), and the percentage of cells containing periphery-localized loci increased from 52% (n=69) to 85% (n=131, p<0.0001) (FIG. 19). Similarly, another single sgRNA (sgCXCR4-2) increased localized CXCR4 loci from 25% (n=202) to 47% (n=284), and cells from 49% (n=74) to 82% (n=84, p<0.0001) (FIG. 19). Efficiency when targeting the CXCR4 locus using single sgRNA and a pool of 6 sgRNAs was compared. When using multiple sgRNAs, the CRISPR-GO system increased the percentage of localized CXCR4 loci from 32% (n=270) to 62% (n=402, p<0.0001) and the percentage of cells from 46% (n=170) to 90% (n=168, p<0.0001), respectively (FIGS. 15 and 19). When using a non-targeting sgRNA as a control, the percentage of CXCR4 loci at the nuclear periphery was similar to that seen with non-ABA treated samples and remained unchanged after ABA treatment (FIG. 16). These results together confirmed that the systems disclosed herein can mediate efficient re-localization of non-repetitive loci to the cellular compartments such as the nuclear periphery.

### Example 4. Chemically Inducible and Reversible CRISPR-GO-Mediated Genomic Repositioning

One advantage of the provided systems and methods is the ability to easily switch on or off polynucleotide re-positioning by adding or removing a chemical inducer to the culture medium. Chemical induction and removal experiments were performed to study the dynamics and reversibility of the ABA-inducible CRISPR-GO system (FIG. 20). To study chemical induction, U2OS cells containing the CRISPR-GO system targeting Chr3 loci were treated with ABA and examined at different time points. For chemical reversal, U2OS cells containing the CRISPR-GO system targeting Chr3 loci were first treated with ABA for 2 days, and then switched to medium without ABA. ABA-induced genomic re-localization occurred relatively quickly as the percentage of Chr3 loci tethered to nuclear membrane increased from 19% (n=142) to 75% (n=93, p<0.0001) within 16 hours of ABA addition, and reached 91% (n=160) after 72 hours of ABA addition. After ABA removal, the percentage Chr3 loci tethered to nuclear membrane decreased from 82% (n=163) to 45% (n=84, p<0.0001) after 24 hours, and further decreased to 27% (n=146) and 26% (n=159) after 48 hours and 72 hours, respectively. After 48 hours and 72 hours of ABA removal, the percentage of Chr3 at the nuclear periphery was indistinguishable from a control sample with no ABA treatment (25%, n=106, p=0.77) imaged at the same time, suggesting that ABA removal fully reversed the genomic repositioning effects mediated by the CRISPR-GO system within 48 hours (FIG. 20). These results suggest that nuclear repositioning mediated by the systems and methods disclosed herein can be easily switched on and off by, for example, adding or removing a chemical inducer such as ABA.

### Example 5. Mitosis-Dependent and Mitosis-Independent Mechanisms for CRISPR-GO System-Mediated Nuclear Periphery Repositioning

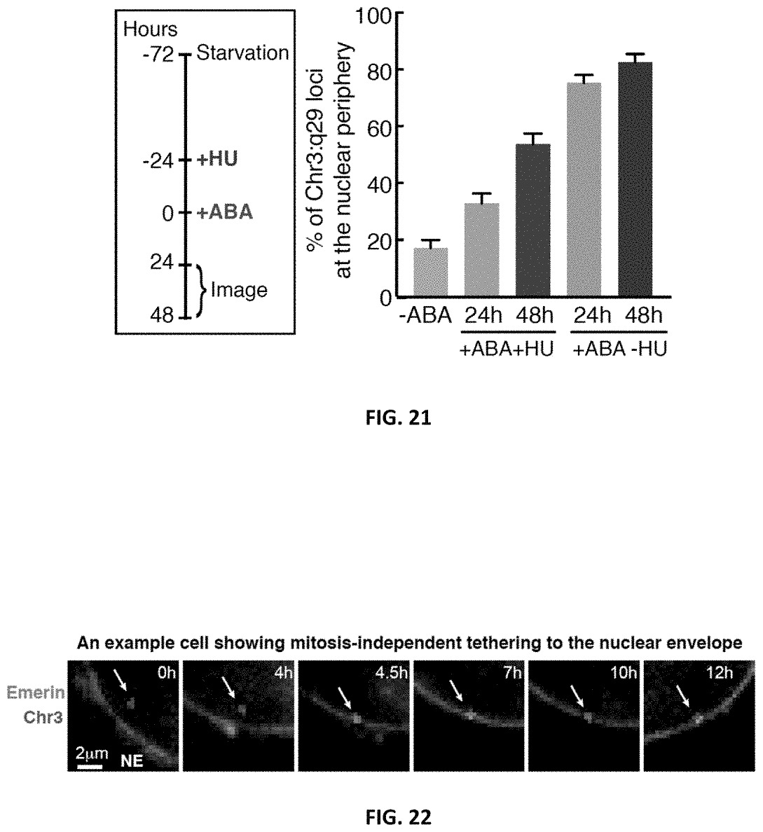

The CRISPR-GO system was used to target the endogenous Chr3 locus. CRISPR-GO cells containing Chr3-targeting sgRNAs were synchronized and arrested in the S phase by serum starvation and Hydroxyurea (HU) treatment and then treated with ABA for chemical induction (FIG. 21). Interestingly, after 24 hours of ABA treatment, the percentage of nuclear periphery tethered Chr3 loci increased from 17% (n=175) to 33% (n=177, p=0008) in HU treated S-phase arrested cells, which was significantly lower than the percentage in unsynchronized cells (75%, n=251, p=0.0001). After 48 hours of ABA treatment, the percentage of nuclear periphery tethered Chr3 loci increased to 54% (n=177, p<0.0001) in HU treated S-phase arrested cells, which is also lower than in unsynchronized cells (83%, n=178, p<0.0001). Thus, in HU treated S-phase arrested cells, nuclear periphery repositioning was still observed, but to a less extent compared to cells that underwent mitosis. These results suggest that repositioning of endogenous loci to the cellular compartments such as the nuclear periphery using the systems and methods disclosed herein may happen via both mitosis-dependent and mitosis-independent mechanisms.

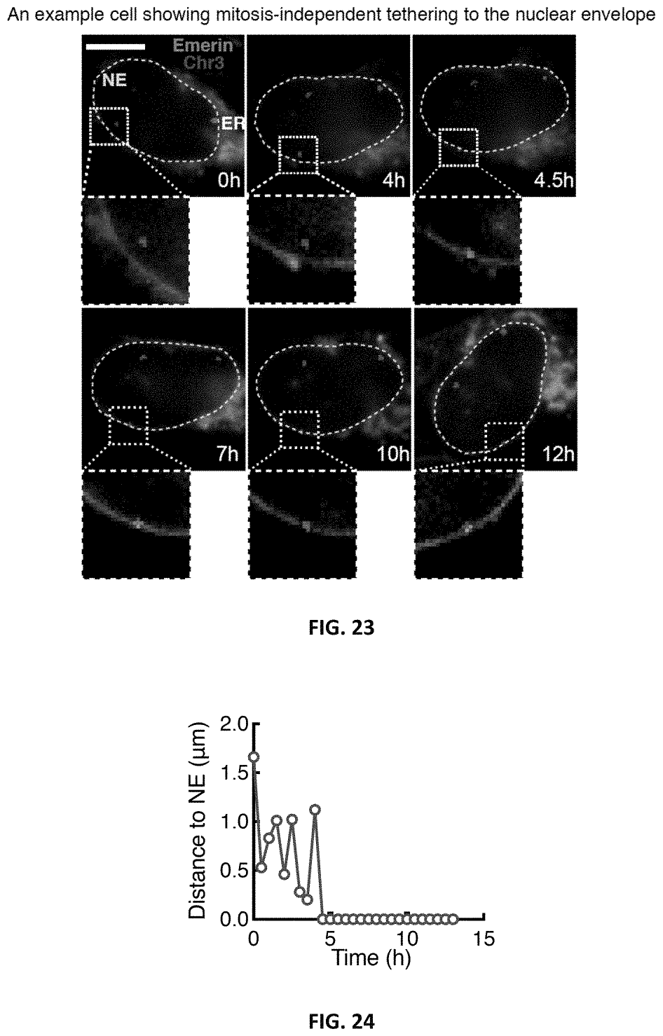

Mitosis-independent periphery repositioning has not yet been reported to our knowledge. To probe whether a genomic locus can be tethered to the nuclear periphery during interphase, live-cell CRISPR-Cas9 imaging was used to track the dynamics of nuclear periphery tethering. The dynamic process of endogenous Chr3 loci becoming tethered to nuclear periphery during interphase was detected. In the representative example shown in FIGS. 22 and 23, a Chr3:q29 locus (arrow) started off separate from the nuclear periphery (GFP-Emerin) during the first 4 hours of recording, became tethered to the nuclear periphery at 4.5 hours and then stayed tethered for the remaining 8 hours of recording, even while the nucleus underwent a rotation between 10 hours and 12 hours. We quantified the distance between this Chr3 locus and the nearest nuclear periphery over time, and found that the distance stayed as 0 after tethering occurred at 4.5 hours (FIG. 24). These results suggest that, despite the relatively stable organization of chromatin structure during the interphase, a genomic locus located close to the nuclear periphery is able to be synthetically tethered to the nuclear periphery in a mitosis-independent manner using the systems and methods disclosed herein.

### Example 6. Suppression of Movements of Endogenous Genomic Loci by Tethering at the Nuclear Periphery

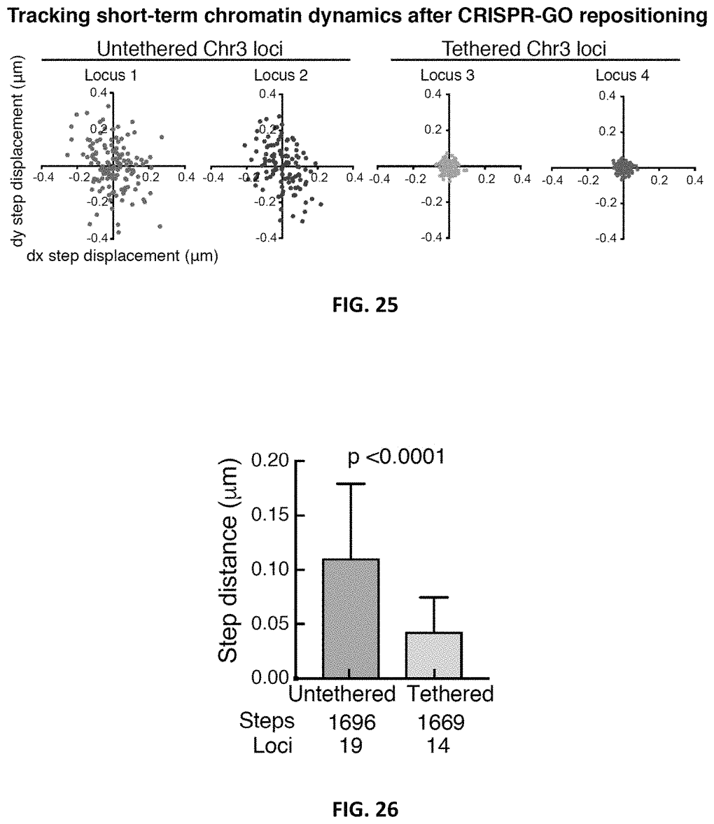

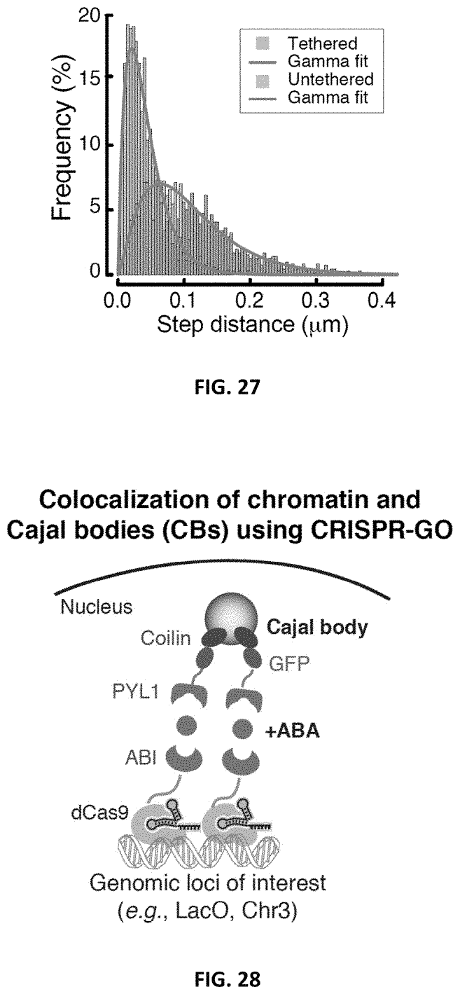

The short-time movement kinetics of genomic loci after genomic tethering was studied by combining the CRISPR-GO system with CRISPR-Cas9 imaging in living cells. The short-term dynamics of Chr3 loci tethered at the nuclear periphery were examined as compared to untethered loci (FIG. 25). Images were taken every 4-6 s under a confocal microscope. We observed that the untethered Chr3 loci were more mobile than the tethered Chr3 loci (FIG. 25). We characterized the displacement between the consecutive movement steps of each locus in a 2-dimensional space (dx^{t}=x^{t}−x^{t-1} & dy^{t}=y^{t}−y^{t-1}, where (x^{t}, y^{t}) is the coordinate of a locus at time t), and plotted their distribution as a measure of movement amplitude (FIG. 25). The untethered Chr3 loci displayed a broader distribution of step displacement with higher amplitude compared to the periphery-tethered loci. The observation that the tethered genomic loci exhibited more confined movement confirms the physical tethering of these loci to the nuclear envelope. We further quantified the average Euclidean step distance (\sqrt{(x^{t}−x^{t-1})²+(y^{t}−y^{t-1})²}). We found that untethered Chr3 loci showed a step distance of 0.11±0.07 μm (1696 steps for 19 loci), and the periphery-tethered Chr3 loci presented a much lower step distance of 0.04±0.03 μm (1669 steps for 14 loci) (p<0.0001, FIG. 26). The step distances of the untethered and tethered loci can both be well approximated by distinct gamma distributions (FIG. 27). These results suggest that cellular compartment tethering mediated by the systems and methods disclosed herein can suppresses the mobility and dynamics of targeted polynucleotides.

### Example 7. Colocalization of Genomic Loci with Membraneless Nuclear Bodies Including Cajal Bodies and PML Bodies

Whether the CRISPR-GO system can mediate colocalization of chromatin loci with membraneless nuclear bodies was next tested. Genomic loci were chosen to recruit to Cajal bodies (CBs). To do this, a Cajal body-targeting CRISPR-GO system was designed by fusing PYL1 with Coilin, a marker of Cajal bodies. PYL1-GFP-Coilin and ABI-dCas9 were introduced into U2OS cells via lentiviral transduction (FIG. 28). We tested the recruitment efficiency in the U2OS 2-6-3 cells containing a LacO repeat array inserted in Chr1:p36.

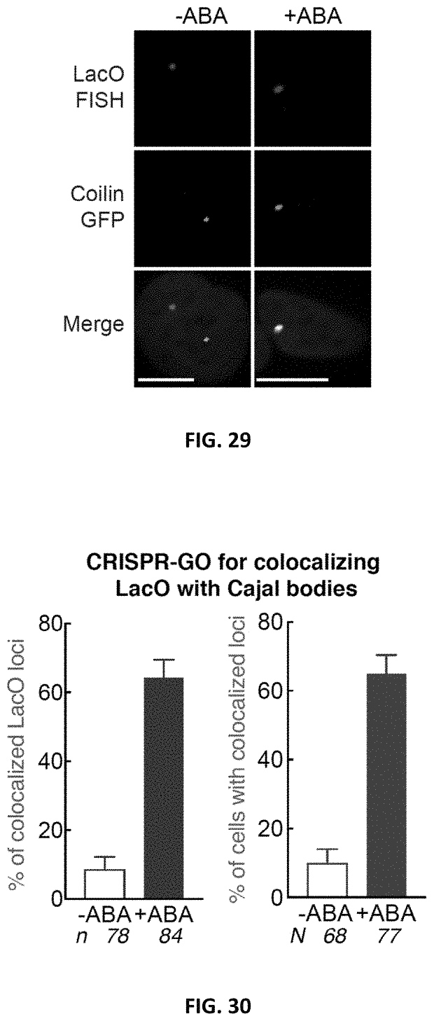

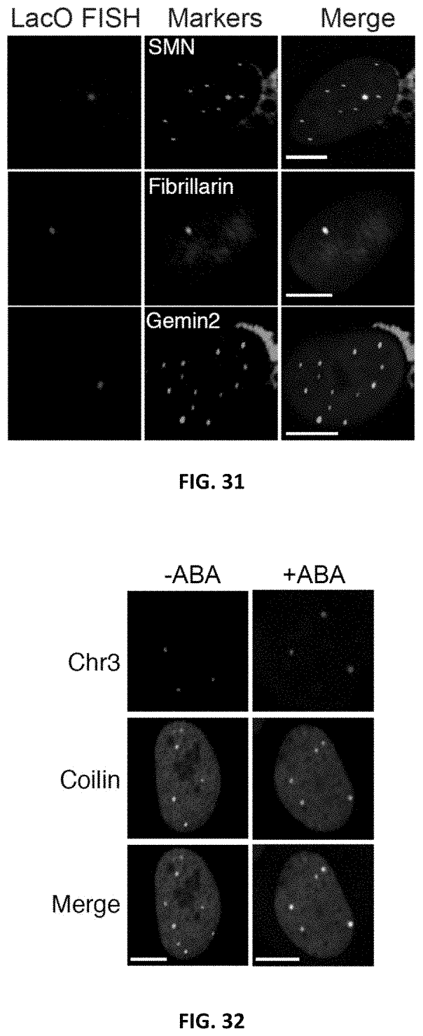

Using an sgRNA targeting the LacO sequence, we visualized the spatial positioning of the LacO array using 3D-FISH and the location of CBs by GFP-Coilin after 2 days of ABA treatment (FIG. 29). The percentage of LacO loci that colocalized with GFP-Coilin-labeled CBs increased from 9% (n=78) without ABA treatment to 64% (n=84) after ABA treatment, and the percentage of cells containing at least one CB colocalized with a LacO locus increased from 10% (n=68) to 65% (n=77) (FIG. 30, p<0.0001). Combined immunostaining with FISH showed that other CB components (SMN, Fibrillarin, and Gemin2) also colocalized with LacO loci after 20 hours of ABA treatment (FIG. 31), confirming CRISPR-GO-mediated colocalization of the targeted genomic loci with CBs.

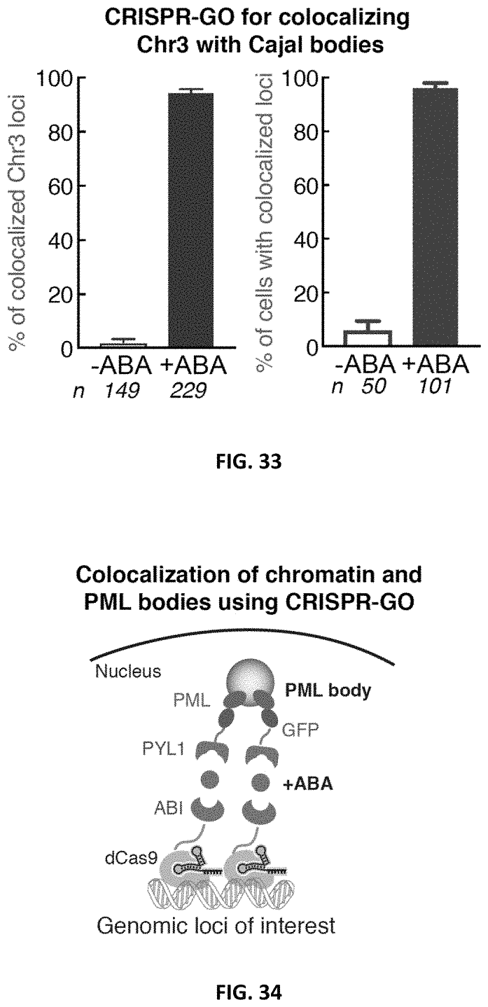

To target endogenous genomic loci to CBs, the Chr3:q29-targeting sgRNA was introduced into U2OS cells expressing the Cajal body-targeting CRISPR-GO system. Significant colocalization was observed between the Chr3 loci (visualized with CRISPR-Cas9 imaging) and CBs (visualized with GFP-Coilin) 24 hours after ABA treatment (FIG. 32). The percentage of Chr3 loci that colocalized with CBs increased from 2% (n=149) before ABA treatment to 94% (n=229, p<0.0001) after ABA treatment, and the percentage of cells containing at least one CB colocalized with a Chr3 locus increased from 6% (n=50) to 96% (n=101, p<0.0001) (FIG. 33).

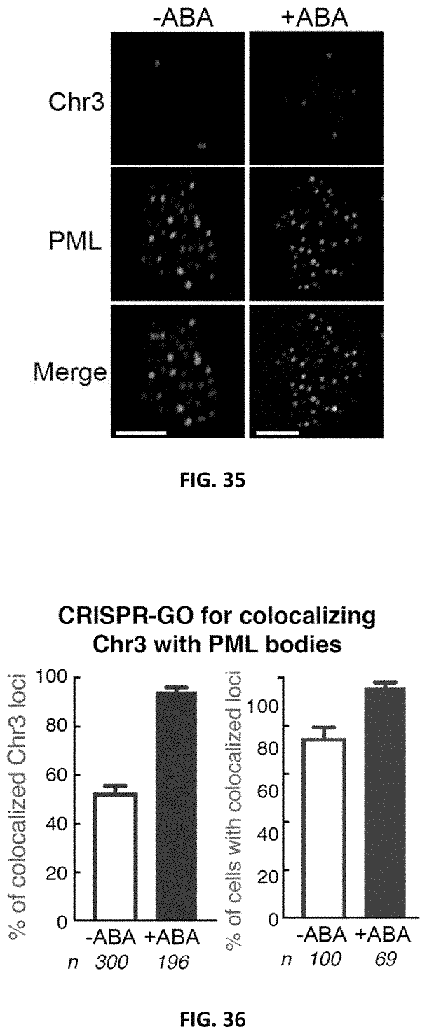

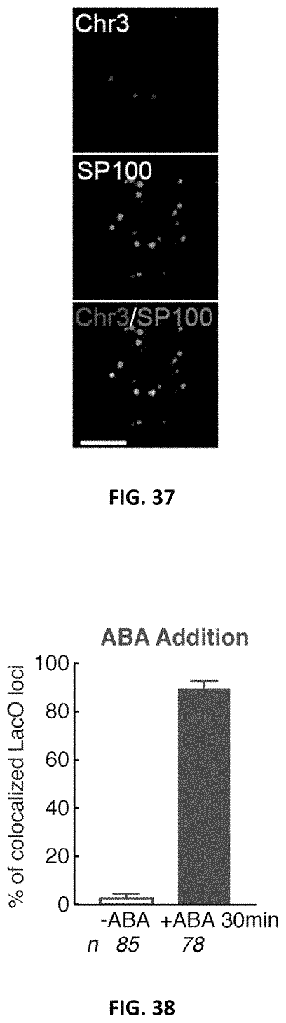

Whether CRISPR-GO could mediate colocalization of chromatin loci with PML nuclear bodies was also tested. To do this, a PML body-targeting CRISPR-GO system was designed by fusing PYL1 with the PML gene, the scaffold protein of PML bodies. To target endogenous genomic loci to PML bodies, the Chr3:q29-targeting sgRNA was introduced into cells expressing both PYL1-GFP-PML and ABI-dCas9, the positioning of Chr3 loci was visualized by CRISPR-Cas9 imaging and the position of PML bodies was visualized by GFP-PML (FIGS. 34 and 35). Interestingly, a high percentage (52.6%, n=300) of Chr3:q29 loci colocalized with the PML bodies without ABA treatment, which may suggest natural Chr3:q29-PML body colocalization (FIGS. 35 and 36). After ABA treatment, the percentage of target Chr3 loci that colocalized with PML bodies increased to 94% (n=196, p<0.0001), and the percentage of cells containing at least one PML body colocalized with a Chr3 locus increased from 75% (n=100) to 96% (n=69, p=0.0003) (FIGS. 35 and 36). Immunostaining also confirmed that Chr3 loci colocalized with SP100, another PML body marker (FIG. 37).

### Example 8. Rapid, Inducible, and Reversible CRISPR-GO Mediated Association Between Target Genomic Loci and Cajal Bodies

Chemical induction and removal experiments were performed to study the dynamics and reversibility of the CRISPR-GO mediated chromatin colocalization with CBs. Using the LacO locus inserted at Chr1:p36 in U2OS 2-6-3 cells as an example, we observed that the association between LacO loci and GFP-Coilin-marked CBs occurred rapidly: within 30 minutes after ABA addition, the percentage of LacO loci that colocalized with CBs increased from 2.6% (n=78) to 89% (n=85, p<0.0001) (FIG. 38).

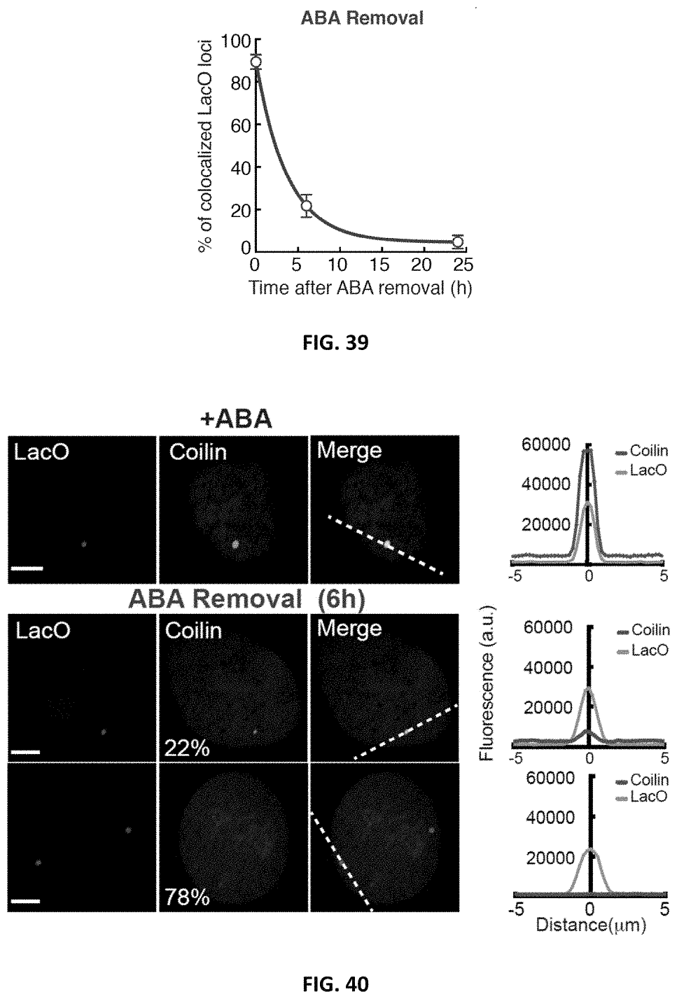

In cells pretreated with ABA for 1 day, ABA removal was observed to lead to dissociation of CBs from LacO loci. After ABA removal, the percentage of the targeted LacO loci that colocalized with CBs decreased from 89% (n=85) to 22% (n=60, p<0.0001) after 6 hours and further decreased to 4.6% (n=45, p<0.0001) after 24 hours (FIG. 39). At 6 hours after ABA removal, among the cell population (22%) that still possessed LacO-colocalized GFP-Coilin, the remaining GFP-Coilin intensity was much dimmer than that in cells undergoing sustained ABA treatment (FIG. 40), which may suggest a gradual disassembly process of CBs after ABA removal.

### Example 9. De Novo CB Formation and Repositioning of Existing CBs at Targeted Chromatin Loci

To further characterize the dynamics of CRISPR-GO-mediated association of Cajal bodies with targeted genomic loci, time-lapse microscopic imaging of individual cells was performed before and after ABA treatment. Theoretically, colocalization between a genomic locus and nuclear bodies could occur through de novo formation of a nuclear body at the genomic locus, or through repositioning the genomic locus to an existing nuclear body. Previous reports using the LacO-LacI tethering system suggest that Cajal bodies form de novo at the targeted DNA site.

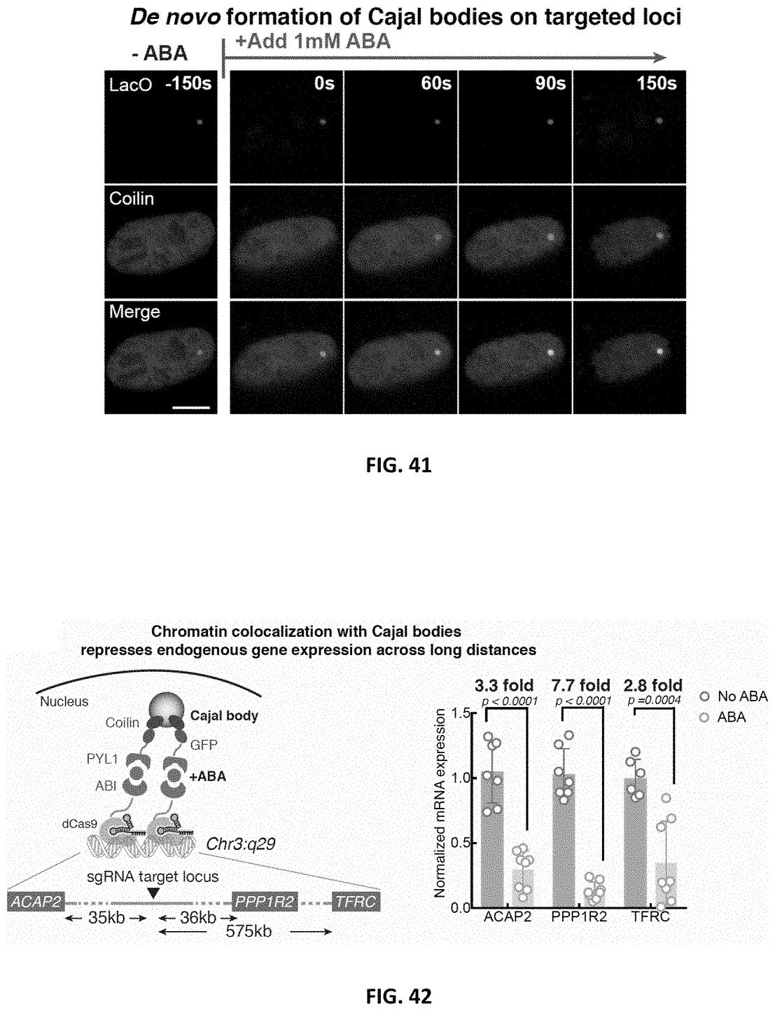

Using the CRISPR-GO system to target LacO loci to Cajal bodies, rapid (within minutes) de novo CBs formation was observed at the LacO locus in most analyzed cells after addition of ABA (FIG. 41). For example, in a cell with no initial GFP-Coilin accumulation at a LacO locus without ABA (FIG. 41, −150 s), ABA treatment (added between −150 s and 0 s, FIG. 42) rapidly recruited GFP-Coilin to the LacO locus (FIG. 41, 150 s), leading to de novo formation of Cajal bodies. The GFP-Coilin fluorescence intensity at the LacO loci approached saturation within 10 minutes after ABA addition (FIG. 44).

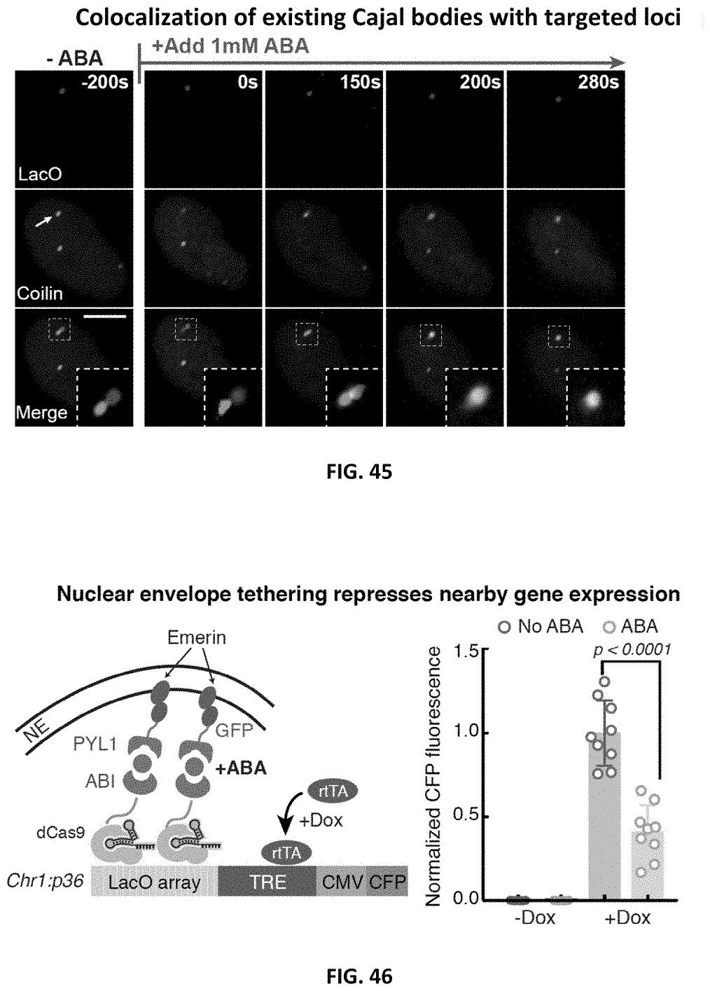

Interestingly, dynamic repositioning of the targeted chromatin locus with an existing CB was observed if the two were initially spatially close to each other. For example, in a cell where an existing CB was adjacent to a LacO locus without ABA treatment (FIG. 45, −200 s), ABA treatment (added between −200 s and 0 s, FIG. 45) led to rapid colocalization of the existing CB and the LacO locus, suggesting that the systems disclosed herein can also mediate direct association between polynucleotides (e.g., genomic loci) and cellular compartments (e.g., existing nuclear bodies), a phenomenon that has not yet been reported before.

### Example 10. Reduced Reporter Gene Expression Resulting from CRISPR-GO-Mediated Relocalizing of Genomic Loci to the Nuclear Periphery

Previous studies offer different evidence about the effect that genomic relocalization to the nuclear periphery has on gene expression. Some studies showed that tethering LacO repeats to the nuclear periphery using LacI-Emerin or LacI-Lap2β caused repression of adjacent gene. Other studies showed re-localization of the LacO array to the nuclear periphery using a LacI-Lamin B1 fusion protein in the U2OS 2-6-3 cells, but observed no obvious changes in adjacent gene expression. CRISPR-GO offers another way to study this question, since it is much easier to test the effects of recruiting different chromosome loci to the nuclear periphery.

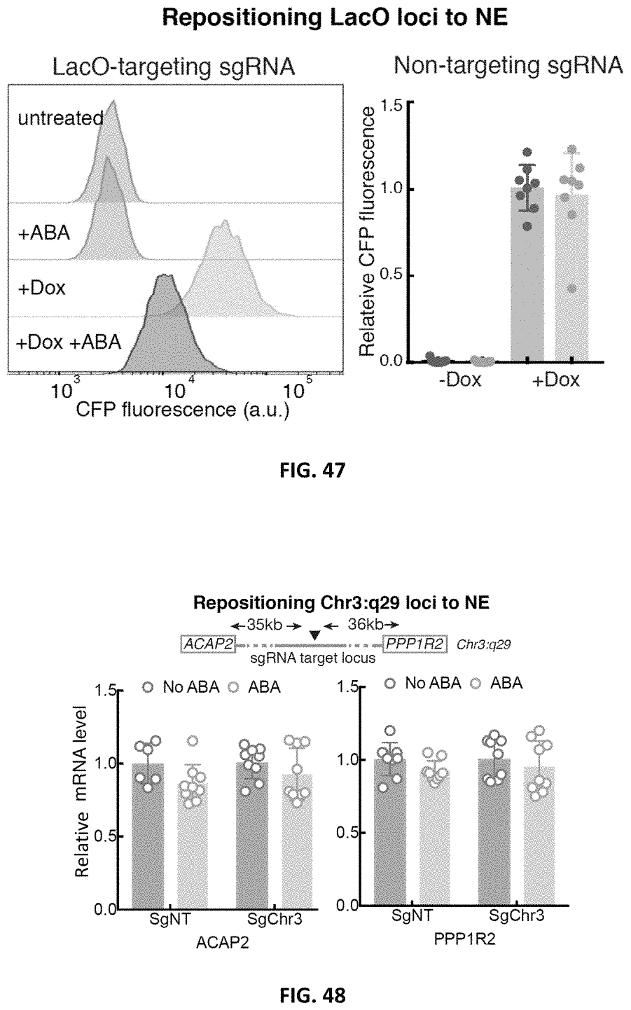

Whether CRISPR-GO-mediated repositioning of the LacO array to the nuclear periphery could influence gene expression was examined. The LacO locus in the U2OS 2-6-3 cells is located upstream of a Doxycycline (Dox)-inducible TRE (Tetracycline responsive element)-CMV promoter that drives expression of a CFP reporter (FIG. 46). The adjacent CFP reporter expression in both ABA-treated and untreated cells were measured by flow cytometry, and ABA-treated cells were observed to show consistently decreased reporter gene expression compared to untreated cells (a reduction of 59%, FIGS. 46 and 47). This gene repression effect was similar to repositioning the LacO locus to the nuclear periphery using a LacI-Emerin fusion protein. As a control to confirm that gene repression was target-specific, we also tested a non-targeting sgRNA, and observed no decrease of the reporter gene expression (FIG. 47).

Whether repositioning endogenous genomic loci to the nuclear periphery could alter gene expression was next tested. The Chr3, XIST, and CXCR4 loci were repositioned to the nuclear periphery individually, and RT-qPCR was performed to detect changes in adjacent gene expression (Chr3: ACAP2 & PPP1R2; CXCR4; XIST). Surprisingly, no evidence of gene expression change was seen for these genes (e.g., ACAP2 & PPP1R2 in FIG. 48). Thus, it raises questions whether repositioning a gene adjacent to the nuclear periphery is sufficient to cause endogenous gene expression changes, and it remains possible that repositioning-induced gene expression changes are locus-dependent.

### Example 11. Reduced Reporter Gene Expression Resulting from CRISPR-GO-Mediated Relocalizing of Genomic Loci to CBs

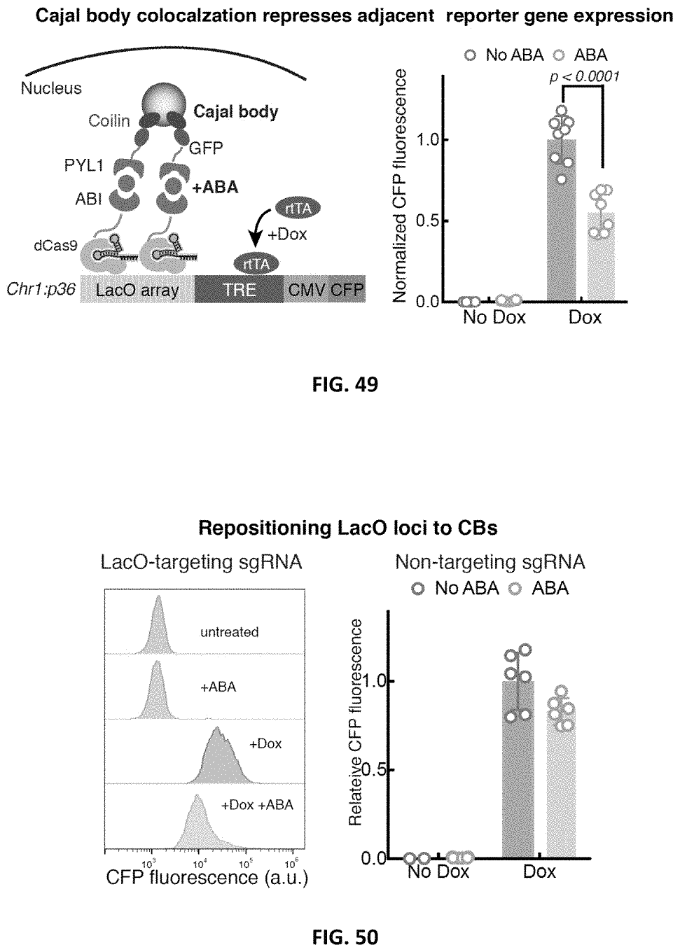

Whether colocalization of LacO loci to CBs using the CRISPR-GO system in the U2OS 2-6-3 cell line was sufficient to influence adjacent gene expression was next tested (FIG. 49). Cells were treated with ABA for 2 days, induced with Dox for 1 day, and measured the CFP expression by flow cytometry. Consistently decreased reporter gene expression was observed in ABA-treated cells compared to untreated cells (an average reduction of 45%, FIGS. 49 and 50, p<0.0001). To confirm this gene repression effect is target-specific, a non-targeting sgRNA was tested, and a slight but not significant decrease (p>0.05) of the reporter gene expression was observed (FIG. 50).

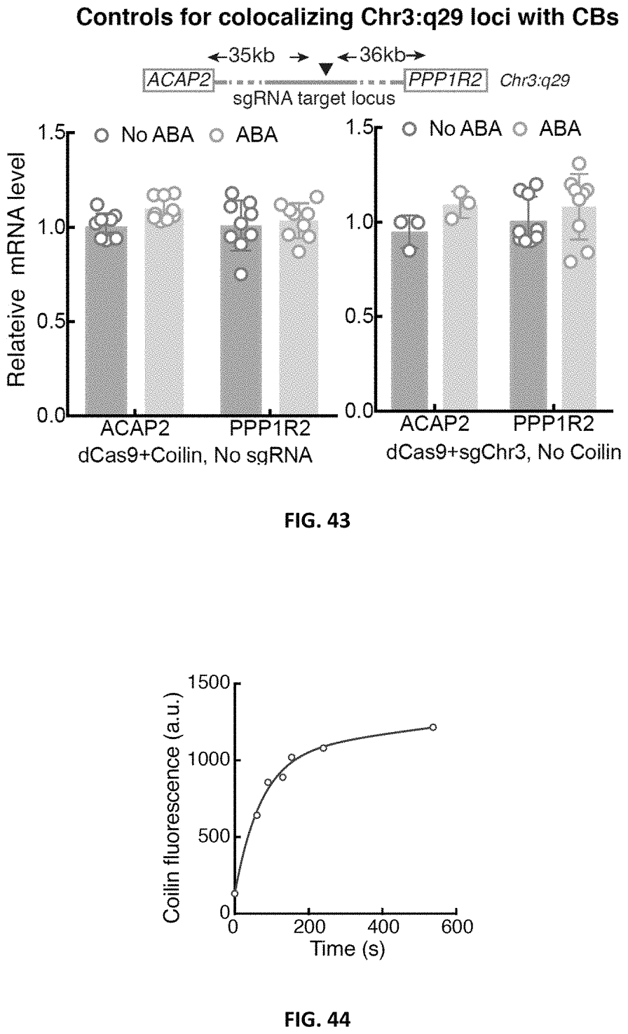

Whether colocalizing an endogenous genomic locus to CBs could alter adjacent gene expression was next tested. The CRISPR-GO system was used to induce colocalization of Chr3:q29 with CBs (FIGS. 32 and 33) and then RT-qPCR was performed to detect changes in adjacent gene expression (Chr3: ACAP2 & PPP1R2) after 4 days of ABA treatment. Surprisingly, significant repression of both adjacent genes compared to untreated cells was observed. ACAP2, located about 35 kb upstream of the CB-targeting loci on Chr3, exhibited 3.3 fold of repression after ABA treatment (p<0.0001, FIG. 42), and PPP1R2, located about 36 kb downstream of the CB-targeting loci on Chr3, exhibited 7.7 fold of repression (p<0.0001, FIG. 42). Cells without sgRNAs or without the PYL-GFP-Coilin component were confirmed as showing no changes in ACAP2 and PPP1R2 gene expression (FIG. 43). Together, these results show that targeted colocalization of a given polynucleotide (e.g., genomic locus) with a cellular compartment (e.g., CBs) is able to repress adjacent polynucleotide expression. The long-distance efficacy of, for example, gene expression perturbation mediation using the systems and methods disclosed herein stands in contrast to CRISPRi or CRISPRa, which only cause perturbations in gene expression a relatively short distance away from the dCas9 binding site. The observation that the provided methods and systems are able to mediate long-distance polynucleotide expression perturbations may provide a useful new means of, for example, gene regulation.

### Example 12. Altered Cellular Phenotypes Resulting from CRISPR-GO-Mediated Telomere Repositioning

The CRISPR-GO system was used to investigate how telomere reorganization to nuclear compartments affected cellular phenotype. Among all genomic loci tested, the dynamics of telomeres are the best studied and are shown to be associated with the nuclear periphery and CBs at certain stages of the cell cycle. Given the important role of telomeres for genome integrity, their interactions with nuclear compartments may have functional implications. For example, during the cell cycle, telomeres are dynamically tethered to the nuclear envelope when the nuclear membrane reassembles in post-mitotic cells, and then relocate to the interior of the nucleus during the G1 phase, where they remain for the rest of cell cycle. The cycle of telomere tethering and untethering to the nuclear envelope may be important for chromatin organization and the cell cycle/viability.

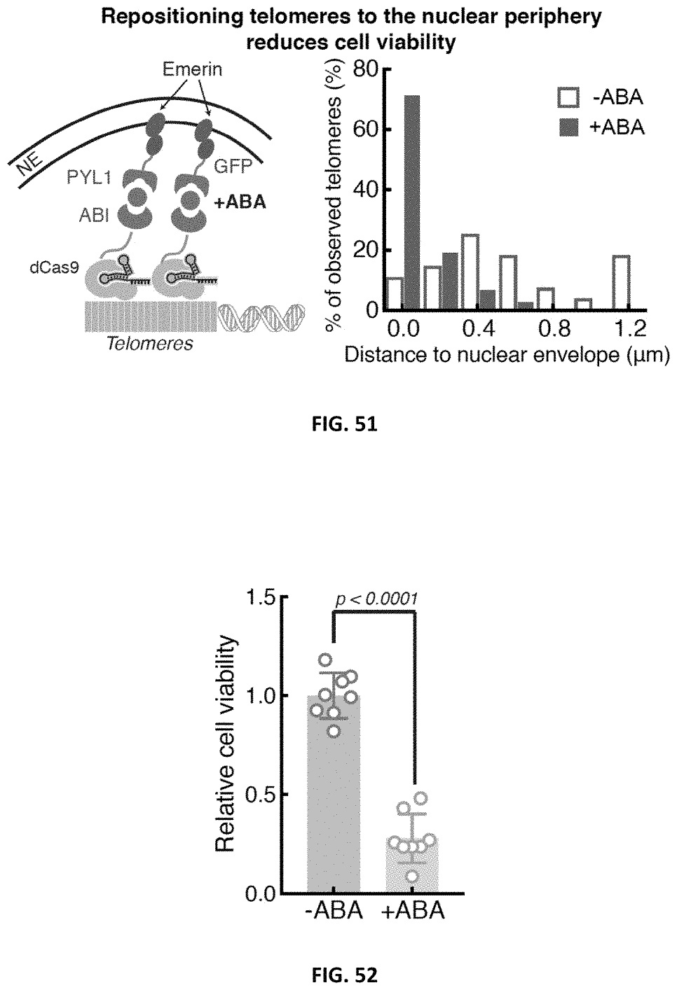

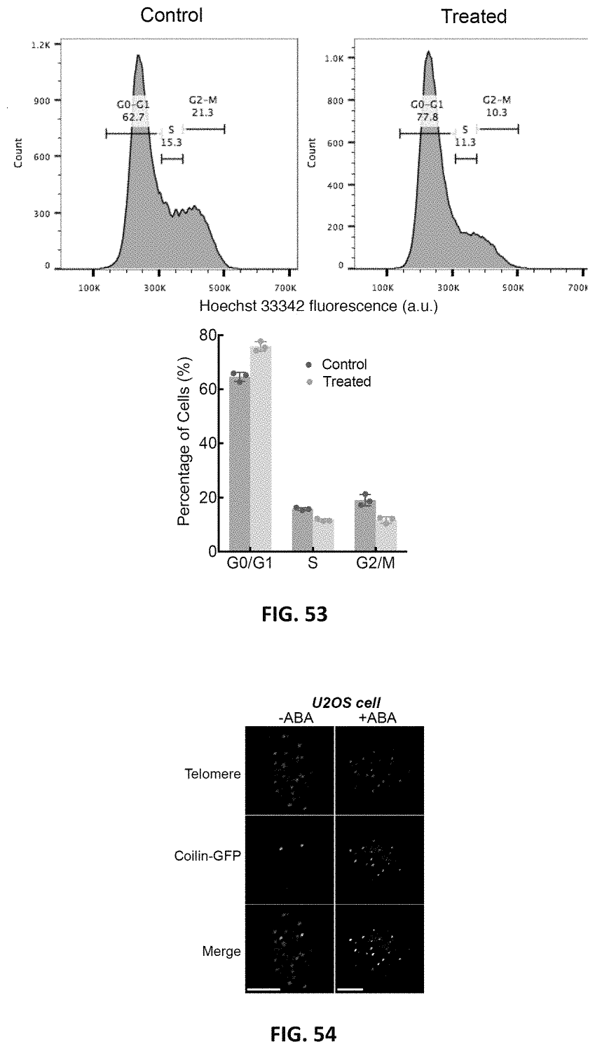

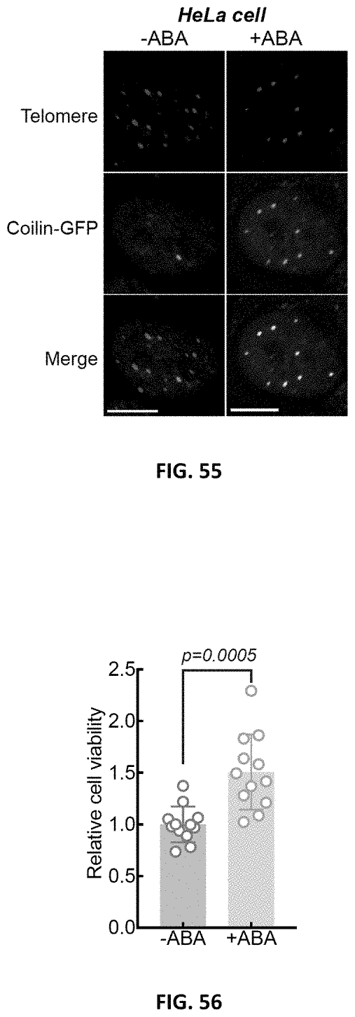

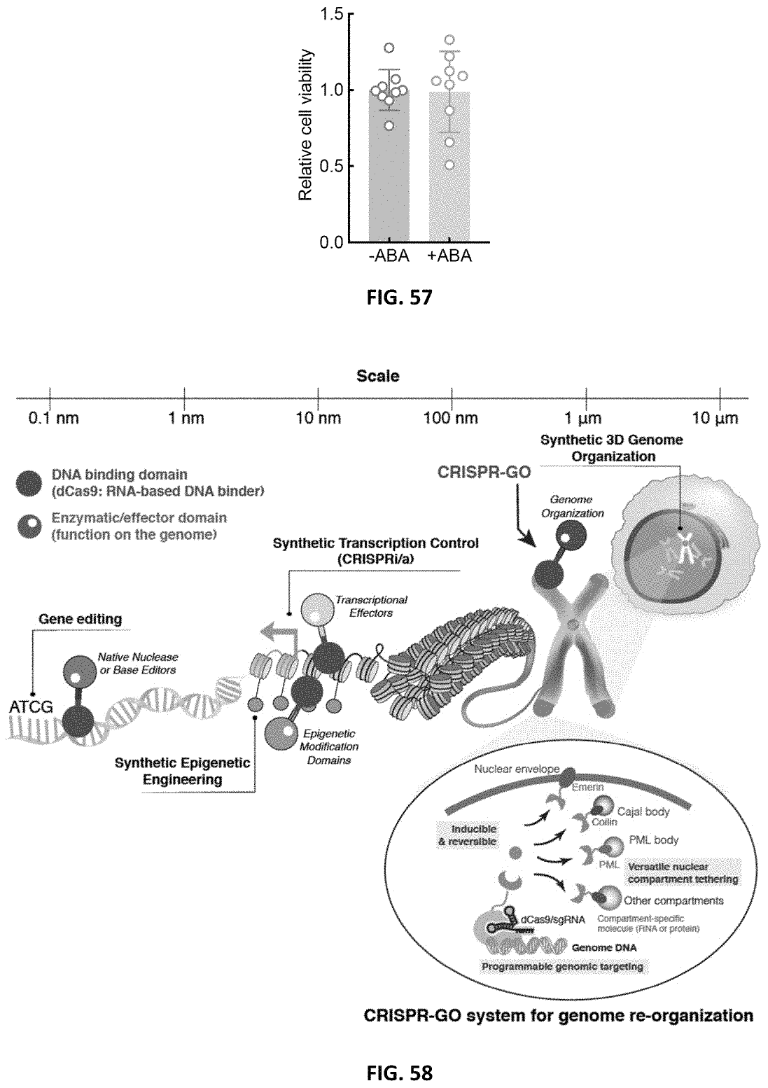

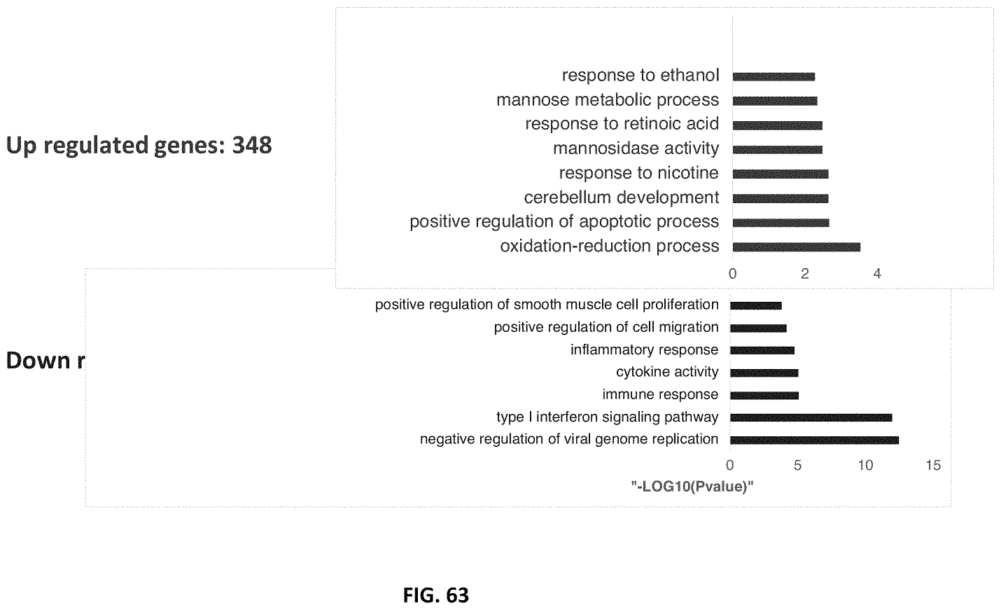

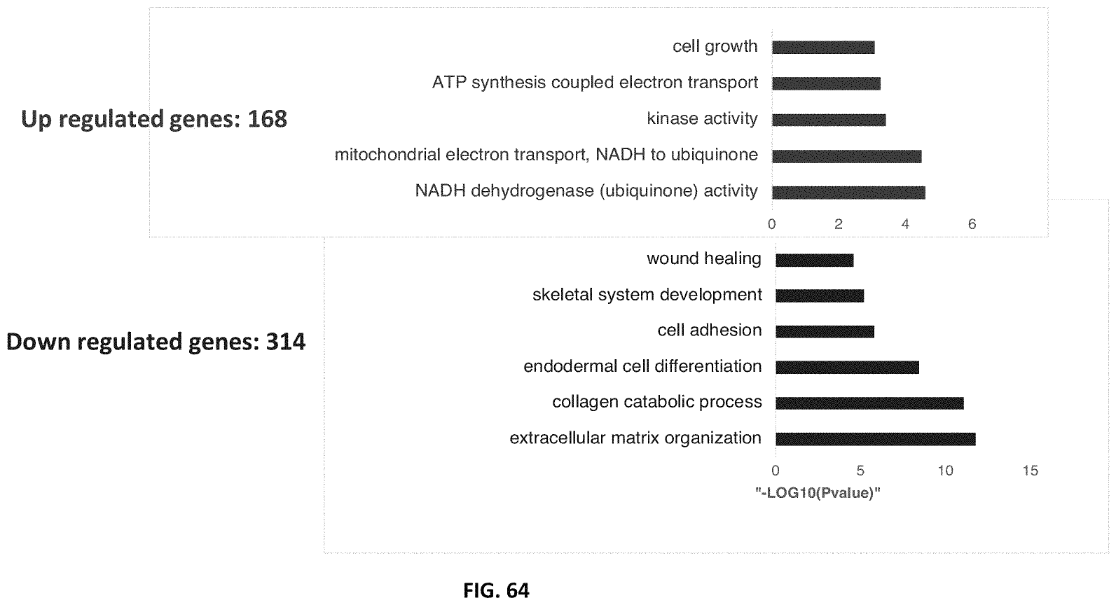

To test this, the CRISPR-GO system was used to disrupt the telomere untethering process during the cell cycle and retain telomeres to the nuclear compartments during interphase (FIG. 51). Using an Alamar blue cell viability assay, which quantifies cell proliferation by measuring metabolic activity of cells, the maintenance of telomeres at the nuclear periphery by CRISPR-GO was found to lead to a significant decrease in cell viability after 6 days of ABA treatment, when compared to untreated cells (FIG. 52, average 72% of reduction, p<0.0001). After 3 days of ABA treatment, cell cycle analysis showed that tethering telomeres to the nuclear periphery increased the percentage of cells in the G0/G1 phase and reduced the percentage of cells in the S phase and the G2/M phase, likely suggesting a G0/G1 phase arrest (FIG. 53). FIG. 63 presents a graph comparing the gene expression changes by RNA sequencing after repositioning telomeres to the nuclear periphery and shows that repositioning telomeres to the nuclear periphery caused many changes in gene expression that reduced cell viability. The effect of colocalizing telomeres with CBs was also examined, confirming that the CRISPR-GO system was able to induce colocalization of telomeres and CBs, and finding that the colocalization increased cell viability when comparing cells treated with ABA for 2 days to untreated cells (average 50% increase, FIGS. 54-56). FIG. 64 presents a graph comparing the gene expression changes by RNA sequencing after co-localizing telomeres with Cajal bodies and shows that co-localizing telomeres with Cajal bodies caused many changes in gene expression that altered cell viability. As a control, ABA treatment alone has no effect on cell viability in U2OS cells (FIG. 57). Altogether, these observations suggest that the spatial organization of polynucleotides (e.g., telomeres) relative to various cellular compartments (e.g., nuclear compartments) plays an important role in cellular function.

### Example 13. Repositioning mRNAs Along the Cytoskeleton Using the CRISPR-GO System

The CRISPR-GO system can be used in repositioning mRNAs along the cytoskeleton with motor proteins such as kinesin, dynein, and myosin. To reposition mRNAs to the plus ends of microtubules (MT+), a plasmid expressing PYL1-EGFP-tagged kinesin-1 heavy chain (KIFSB) without the cargo binding tail domain can be constructed (Kapitein et al., 2010). To reposition mRNAs to the minus ends of microtubules (MT−), a plasmid expressing PYL1-EGFP-tagged N-terminal portion of Bicaudal D2 (BICDN), which induces dynein-mediate cargo transport, can be constructed (Hoogenraad et al., 2003). To reposition mRNAs along actin filaments (AF), a plasmid expressing PYL1-EGFP-tagged myosin 5a (MYO5A) can be constructed. MYO5A is the best characterized of the three class V myosins and plays a role in the transport of mRNA along actin filaments towards the barbed end (Gross et al., 2007; McCaffrey and Lindsay, 2012). A plasmid expressing ABI-BFP-dCas13 and plasmids expressing PYL1-EGFP-KIFS/BICDN/MYO5A can be transduced into MS2-MCP (MS2-binding protein) cells. Cells are subsequently sorted for BFP and EGFP positive cells to create the MS2-MCP-CRISPR-GO-MT+/MT−/AF stable cell lines. The stable cells can be transduced with lentivirus expressing gRNAs targeting MS2-tagged RNA, and gRNA-positive cells are selected with puromycin. The selected cells can be treated with ABA and perform live-cell fluorescence imaging to track the localization of mCherry, which denotes the position of targeted RNAs.

### Example 14. Formation of Nuclear Bodies to Facilitate DNA Repair and Improve Gene Editing Outcomes Using the CRISPR-GO System

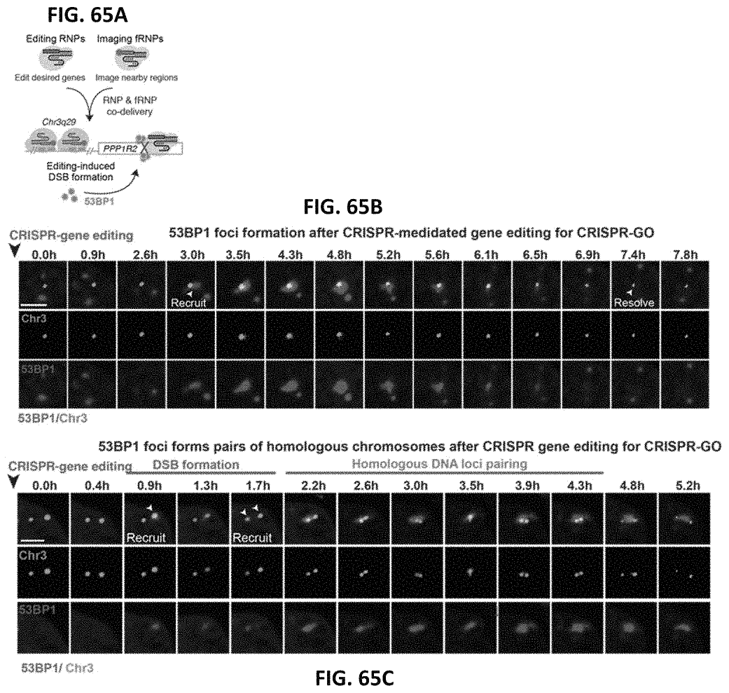

The CRISPR-GO system can be used to form nuclear bodies that facilitate DNA repair and lead to improved gene editing outcomes. FIGS. 65A-65C show the formation of 53BP1 foci after CRISPR-mediated gene editing. These data demonstrate that the CRISPR gene editing recruiting DNA repair proteins form nuclear bodies to facilitate double-strand break (DSB) resolution and DNA repair after CRISPR-mediated gene editing.

### Example 15. Plasmids Collections and Constructions

pHR-SFFV-PYL1-sfGFP-Emerin was cloned by replacing scFv sequence in pHR-SFFV-scFv-sfGFP plasmid (Tanenbaum et al., 2014) with PYL1 and inserting Emerin after sfGFP. Emerin (encoded by the EMD gene) was cloned from Emerin pEGFP-C1 (637), a gift from Eric Schirmer (Zuleger et al., 2011) (Addgene plasmid 61993). pHR-SFFV-PYL1-sfGFP-Coilin was cloned by replacing Emerin in pHR-SFFV-PYL1-sfGFP-Emerin plasmid with Coilin. Coilin was cloned from pEGFP-Coilin (Addgene plasmid 36906), a gift from Dr. Greg Matera. pHR-PGK-PYL1-sfGFP-Coilin was cloned by replacing SFFV promoter in pHR-SFFV-PYL1-sfGFP-Coilin plasmid with PGK promoter. pHR-TRE3G-PYL1-sfGFP-PML or pHR-TRE3G-PYL1-sfGFP-HP1a was cloned by replacing PGK promoter with TRE3G promoter, and replacing Coilin with PML or HP1a in the pHR-PGK-PYL1-sfGFP-Coilin plasmid. PML was cloned from pLPC-Flag-PML-IV (addgene plasmid 62804), a gift from Gerardo Ferbeyre (Vernier et al., 2011). HP1a was cloned from GFP-HP1a (Addgene plasmid 17652), a gift from Tom Misteli (Cheutin et al., 2003).

pHR-SFFV-ABI-tagBFP-dCas9 was described before (Gao et al., 2016). pHR-SFFV-ABI-tagBFP-dCas9 was cloned by replacing SFFV promotor with PGK promoter pHR-SFFV-ABI-tagBFP-dCas9. pHR-PGK-ABI-dCas9-P2A-Cherry, or pHR-PGK-ABI-dCas9-P2A-Puro was cloned by replacing SFFV with PGK promoter, deleting tagBFP and adding P2A-mCherry or P2A-Puro in dCas9 pHR-SFFV-ABI-tagBFP-dCas9. ABI and PYL1 were cloned from Addgene plasmid 38247 (Liang et al., 2011), a gift from Dr. J. Crabtree, Stanford.

pHR-TRE3G-dCas9-HaloTag was cloned by replacing SunTag10-P2A-mCherry with HaloTag in the plasmid pHR-TRE3G-dCas9-HA-SunTag10-P2A-mCherry (Tanenbaum et al., 2014). pHR-TRE3G-dCas9-EGFP-HaloTag was cloned by inserting HaloTag after EGFP in pHR-TRE3G-dCas9-EGFP (Chen et al., 2013). pHR-SFFV-DHFR-mCherry-Emerin was cloned by replacing PYL1-sfGFP sequence in pHR-SFFV-PYL1-sfGFP-Emerin with mCherry-DHFR. HaloTag and mCherry-DHFR was cloned from pERB221, gift from David Chenoweth & Michael Lampson (Ballister et al., 2014) (Addgene plasmid 61502).

All sgRNAs were cloned into pHR-U6-sgTel-CMV-puro-P2A-mCherry vector after removing P2A-mCherry (Chen et al., 2013). TRF1-mCherry was cloned into pHR-U6-sgTel-CMV-puro-P2A-mCherry vector in place of mCherry. TRF1 was cloned from pLPC-NFLAG TRF1, a gift from Dr. Titia de Lange (Smogorzewska and de Lange, 2002) (Addgene plasmid #16058).

### Example 16. Cell Culture and Generation of Stable Cell Lines

The U2OS (human bone osteosarcoma epithelial, female) cells and Hela cells (female) were cultured in DMEM with GlutaMAX (Life Technologies) in 10% Tet-system-approved FBS (Life Technologies). U2OS 2-6-3 cell line was a gift from Dr. David L. Spector in Cold Spring Harbor Laboratory and were cultured in the same condition (Kumaran and Spector, 2008). All cells were cultured at 37° C. and 5% CO2 in a humidified incubator.

To create stable CRISPR-GO cell lines targeting endogenous loci to nuclear compartments, U2OS cells were plated into 24-well plates 1 day ahead to reach 50% confluency, and then transduced by lentivirus mixture. Cells transduced by lentivirus expressing PYL1-sfGFP-Emerin, PYL1-sfGFP-Coilin, PYL1-sfGFP-PML, or PYL1-sfGFP-HP1a and ABI-tagBFP-dCas9 were sorted by fluorescence activated cell sorting (FACS) at Stanford shared FACS facility for cells that are BFP and GFP positive to create stable cell lines. For nuclear periphery tethering, cells of high BFP and GFP expression level was selected. For other nuclear compartment tethering, cells of high BFP and GFP expression level was selected. After transducing CRISPR-GO cell lines with lentivirus expressing targeting sgRNAs, sgRNA-positive cells were selected with puromycin at 2 μg/ml.

To target LacO loci in the U2OS 2-6-3 cell lines (Kumaran and Spector, 2008), cells were transduced by lentivirus mixture containing PYL1-sfGFP-Emerin or PYL1-sfGFP-Coilin and ABI-dCas9-P2A-mCherry. Cells containing PYL1-sfGFP-Coilin and ABI-dCas9-P2A-mCherry were sorted for GFP and mCherry positive cells to created stable cell lines. SgRNAs positive cells were selected with puromycin at 2 μg/ml.

To quantify the efficacy of LacO nuclear periphery repositioning by CRISPR imaging, U2OS 2-6-3 cells were transduced with lentivirus coding ABI-dCas9-P2A-Puro instead of ABI-dCas9-P2A-mCherry, and were selected with puromycin at 2 μg/ml.

### Example 17. Genomic Loci Targeted by CRISPR-GO System

The efficacy of CRISPR-GO system targeting different chromosomal regions in U2OS cells was tested. Both repetitive regions and non-repetitive genes were tested (FIGS. 7, 14, and 17). The endogenous repetitive regions include Chr3.q29: 195478324-195506987; CH13.q34: 112,277485-112,319169; Ch7:q36.3: 158,122,661-158,135,328; ChX p21.2: 30,806,671-30,824,818 and telomeres. A synthetic LacO repeat inserted in Chr1.p36 region in the U2OS 2-6-3 cells was also used for targeting. For repetitive regions, a single sgRNA design was used targeting multiple repeats within the targeted region (Table 1). Non-repetitive genes include CXCR4 located at Chr2.q22.1, XIST located at ChrX.q13.2, and PTEN located at Chr10.q23.31. To target each non-repetitive gene, multiple sgRNAs were designed targeting its gene body and upstream region (Table 2).

TABLE 1
sgRNAs targeting repetitive regions.

| Name | Sequences | Genomic regions |
|---|---|---|
| sgChr3 | TGATATCACAG | Hg38: Chr3: 195478324- |
| | | 195506987 |
| sgChr13 | ACCATTCCTTC | Hg38: CH13: 112277485- |
| | | 112319169 |
| sgChrX | GGCAAGGCAAGGCAAGGCACA | Hg38: ChX: 30,788554- |
| | | 30,806701 |
| sgChr7 | GCTCTTATGGTGAGAGTGT | Hg38: Ch7: 158,329,969- |
| | | 158342636 |
| sgTel | GTTAGGGTTAGGGTTAGG | Telomeres |
| sgLacO-1 | GCTCACAATTCCACATG | Chr1.p36 in U2OS 2-6-3 cells |
| sgLacO-2 | GCCACATGTGGAATTGTGAG | Chr1.p36 in U2OS 2-6-3 cells |
| sgNT | GTACGTTCTCTATCACTGATA | Non-targeting |
| | TGATATCACAG | |

TABLE 2
sgRNAs targeting non-repetitive regions.

| Name | Sequences | Genomic regions |
|---|---|---|
| PTEN | | |
| sgPTEN-1 | GATCAGCTCTCTCACGGTGAC | Chr10: 87,863,113-87,971,930 |
| sgPTEN-2 | GCACTTGGCTGAGTCCACAGT | forward strand |
| sgPTEN-3 | GACATCGGAGAATGCACGCTC | |
| sgPTEN-4 | GAATAGGTCGATGTAGAGCA | |
| sgPTEN-5 | GCCGCGTTCTGTAAGAATCGG | |
| sgPTEN-6 | GTAAGTCCTATGACAGAAGC | |
| sgPTEN-7 | GAGATATACTGTTAGCGCCTT | |
| sgPTEN-8 | GGCTGTAGACATCAATGCTT | |
| sgPTEN-9 | GATCATTGCAGGTAAGAAGTG | |
| XIST | | |
| sgXIST-1 | GCTCCAACACTCTACCTTGTA | Chr X: 73,820,651-73,852,753 |
| sgXIST-2 | GGAAGTGCTTACGAGTCAAT | reverse strand |
| sgXIST-3 | GCTCTGTCAGTAACTGATAAG | |
| sgXIST-4 | GCAAGCTTACCTGATAGATT | |
| sgXIST-5 | GTTCCATCTTCTAAGTGTCCT | |
| sgXIST-6 | GAACTGAGACTTGTGACACA | |
| sgXIST-7 | GAGTTAGAAGTCTTAAGACC | |
| sgXIST-8 | GTTCCATCCAACTCAGGCCTT | |
| sgXIST-9 | GCCTGAGGCTTCTATCTATCT | |
| sgXIST-10 | GGAAGGCTCATATGGATAGA | |
| sgXIST-11 | GATCATCTCACATGGCAGCC | |
| sgXIST-12 | GGCTATCAGAGCAAGCATTG | |
| sgXIST-13 | GTGTGTGTCATGTGTGGCAG | |
| CXCR4 | | |
| sgCXCR4-1 | GCGCATGCGCCGCTGGGGCG | Chr2: 136,114,349- |
| sgCXCR4-2 | GCAGACGCGAGGAAGGAGGGCGC | 136,118,165 reverse strand. |
| sgCXCR4-3 | GGTAGCAAAGTGACGCCGA | |
| sgCXCR4-4 | GCTCCAGTAGCCACCGCATC | |
| sgCXCR4-5 | GCCTATATAGTGCGGGTGGG | |
| sgCXCR4-6 | GTGAGTCGAGGAGAAACGAC | |

### Example 18. Lentivirus Production

To produce lentivirus, HEK293T cells were transiently transfected with pHR constructs of interest, and packaging plasmids pCMV-dR8.91, and PMD2.G. Lentivirus was collected 72 hours after transfection by filtering supernatant through 0.45 μm filters. When necessary, virus supernatant can be concentrated using Lenti-X concentrator at 4° C. overnight, and centrifuged at 1500 g for 30 min at 4° C. to collect virus pellet. The pellets are suspended in cold culture medium, directly added into cells or frozen down in −80° C.

### Example 19. Genomic Loci Visualization Via CRISPR Imaging and FISH

CRISPR imaging was performed to visualize the localization of Chr3, Chr13 and LacO loci in living cells (FIG. 5). For live-cell CRISPR imaging, stable cell lines expressing CRISPR-GO components were transduced with lentivirus coding dCas9-HaloTag and targeting sgRNAs in ibidi 24-well microplate (Ibidi.inc). Targeted genomic loci are labeled by dCas9-HaloTag and stained by JF549-HaloTag ligand at 0.1-0.5 μM for 15 min at 37° C. in culture media. After staining, cells were washed with culture medium twice, and then incubated in phenol-red free culture medium during microscopy. JF549-HaloTag was a gift from Dr. Luke D. Lavis in Janelia Research Campus (Grimm et al., 2015). Telomere loci are labeled in living cells by expression of TRF1-mCherry, a telomere binding protein.

Other genomic loci are labeled by DNA FISH in fixed cells. Cells were grown in ibidi chamber slides with a removable 12 well silicone chamber, and fixed with 4% PFA for 20 minutes. Lac O, Chr7 and ChrX loci were labeled using synthesized fluorescent nucleotide probes (Integrated DNA Technologies, Redwood City, Calif.) according to a FISH protocol described (Takei et al., 2017). LacO loci were labeled with the Alexa Fluor 647 labeled FISH probe 5′-TTGTTATCCGCTCACAATTCCACATGTGGCCACAAA-3′ at 10 nM concentration. Chr7 loci were labeled by Cy3 labeled FISH probe 5′-Cy3-CCCACACTCTCACCATAAGAGC-3′ at 200 nM, and ChrX loci were labeled by 5-Cy3-TTGCCTTGTGCCTTGCCTTGC-3′ at 200 nM. The CXCR4 FISH probe was purchased from Empire Genomics. The PTEN and XIST FISH probes were purchased from Cell Line Genetics. FISH was performed according merchandiser's protocols.

### Example 20. Immunostaining of Nuclear Body Markers

To detect co-localization between Cajal body markers and targeted LacO loci, U2OS 2-6-3 cells expressing a low level of PYL1-sfGFP-Coilin were transfected with lentivirus coding PGK-ABI-dCas9-P2A-Puro and sgLacO on day 0, treated with puromycin and 3 mM ABA on day 1, and fixed on day 2 after 20 hours of ABA treatment. FISH was performed in fixed samples to detect LacO loci using Alexa Fluor 647 labeled FISH probe, and then immunostaining was performed using mouse monoclonal anti-SMN, anti-Fibrillarin and anti-Gemin2 antibody, and Donkey anti-mouse Alex Fluor 594 secondary antibody.

To detect co-localization between PML body markers and targeted Chr3 loci, U2OS cells expressing PYL1-sfGFP-Coilin and PGK-ABI-dCas9 were transfected with lentivirus coding dCas9-HaloTag (for CRISPR imaging) and sgChr3 on day 0, treated with puromycin and 3 mM ABA on day 1, stained by JF549-HaloTag and fixed in 4% paraformaldehyde (PFA) in Day 3. Immunostaining was performed in fixed samples with rabbit polyclonal anti-SP100, and Donkey anti-rabbit Alex Fluor 647 secondary antibody.

For immunostaining, the fixed samples permeabilized in the permeabilization buffer (PBS, 1% Triton-X100) for 15 min, blocked in blocking buffer (PBS, 0.3% Triton-X 100, 5% Donkey normal Serum) for 1 h, incubated with the primary antibody diluted in the blocking buffer overnight at 4° C., washed in PBS three times, then incubated with the secondary antibody at room temperature for 1-2 hours, and washed four times in PBS.

### Example 21. Chemical Induction and Reversal Experiments

For re-localization experiments, U2OS cells containing chemical-inducible re-localization systems and sgRNAs are treated by abscisic acid (ABA, Sigma-Aldrich, A1049) at 3 mM for 2 days before imaging or fixation.

For the time-course chemical induction experiment targeting Chr3 to nuclear periphery, U2OS cells containing CRISPR-GO and CRISPR imaging systems and sgRNAs targeting Chr3 were treated with or without 3 mM ABA, stained by JF549-HaloTag, and fixed at different time points. For the time-course reversal experiment, the Chr3-targeting U2OS cells were pre-treated with 3 mM ABA for 2 days, washed five times, and switched to medium without ABA. Cells were stained by JF549-HaloTag ligand for CRISPR imaging and fixed in 4% paraformaldehyde for 20 min at different time points.

For the time-course chemical induction experiment targeting LacO to Cajal body, U2OS 2-6-3 cells expressing a low level of PYL1-sfGFP-Coilin were transfected with lentivirus coding PGK-ABI-BFP-dCas9 and sgLacO on day 0, treated with puromycin on day 1, treated with or without 3 mM ABA on day 2 and fixed after 30 minutes of ABA treatments. For the time-course reversal experiment, cells were pre-treated with 3 mM ABA for 2 days, washed five times, and switched to medium without ABA. Cells were fixed in 4% paraformaldehyde for 20 min at different time points.

### Example 22. Cell Cycle Synchronization

To dissect mitosis-dependence effect of genomic re-localization, U2OS cells containing CRISPR-GO and CRISPR imaging systems and sgRNAs targeting Chr3 were used for this experiment. On day −3, cells were starved in 0.5% FBS in medium for 2 days. On day −1, cells were switched to normal growth medium with 10% FBS and treated with 2 mM hydroxyurea (HU) for G1/S phase blockage for 1 day. On day 0, while keeping the HU treatment, cells were treated with or without ABA. Control cells were treated in the same way but without HU. Cells were stained by JF549-HaloTag for CRISPR imaging and fixed in 4% paraformaldehyde 24 h or 48 h after ABA treatment.

### Example 23. Microscopy

With the exception of FIG. 22, all microscopy was performed on a Nikon TiE inverted confocal microscope equipped with the 100×PLAN APO oil objective (NA=1.49), 60×PLAN APO oil objective (NA=1.40) or the 60×PLAN APO IR water objective (NA=1.27), an Andor iXon Ultra-897 EM-CCD camera and 405-nm, 488-nm, 561-nm and 642-nm lasers. Images were taken using NIS Elements version 4.60 software by time-lapse microscopy with Z stacks at 0.2-μm or 0.4-μm steps. For live cell imaging, cells were kept at 37° C. and 5% CO2 in a humidified chamber.

For long-term live cell imaging shown in FIG. 22, microscopy was performed in Leica DMI8 inverted microscope equipped with the 63×HC PLAN APO oil objective (NA=1.40), a Leica DFC9000 CT camera and a Lumoncor SOLA SM II 405 light source. Images were taken using LAS X Software by time-lapse microscopy every 30 minutes for 20 hours, using GFP and TXR filter cubes. During imaging, cells were kept at 37° C. and 5% CO2 in a humidified chamber (Okolab Cage incubation system).

To visualize the dynamics of chromatin-Cajal body association in individual cells (FIGS. 38-41, 44, and 45), U2OS 2-6-3 cells expressing a lower level of PYL1-sfGFP-Coilin was transfected with lentivirus coding PGK-ABI-BFP-dCas9 and sgLacO on day 0, treated with puromycin on day 1 and seeded in ibidi 96 well u-plates. Each well was imaged under confocal microscope to focus on a ABI-BFP-dCas9 labeled LacO locus in a chosen cell. Images were captured before ABA treatment for comparison. Without moving the sample under the confocal microscope, 10-fold ABA-containing culture medium was added into the imaging well to reach a final concentration of 1 mM ABA, and then the same cell containing the previously focused LacO locus was immediately imaged after adding the ABA. The first image taken after ABA addition was given t=0, and all other images were aligned by the capture time accordingly.

### Example 24. Imaging Processing and Data Analysis

Image processing was performed in Fiji (image J) (Schindelin et al., 2012) or MetaMorph (Molecular devices, CA). A single microscope plane showing maximum fluorescence of labeled genomic loci, or the average of two/three adjacent Z planes showing maximum loci fluorescence are shown in the drawings herein. Some images were processed using the “smooth” function in Fiji to reduce noises for visualization only.

Line scan was performed using the “Analyze/Plot Profile” function in Fiji, analyzed in Excel and plotted in GraphPad Prism (Version 7.00 for Mac OS, GraphPad Software, La Jolla Calif. USA, www.graphpad.com). Fluorescence intensity at each point along the line were normalized relative to the maximum (=1) and the minimum (=0) fluorescence intensity along the line.

### Example 25. Quantification of Genomic Repositioning Events

To determine the peripheral recruitment efficacy in living U2OS cells, Chr3, Chr13 and Chr1/LacO loci are labeled by CRISPR imaging and telomeres are labeled by TRF1-mCherry, while the nuclear membrane is labeled by PYL1-sfGFP-Emerin. After scanning Z-stacks of confocal planes, the position of each labeled locus is viewed in slice viewer (NIS element viewer) to determine its position in XY, XZ and YZ planes. Without double counting any loci, the loci were categorized into three categories: loci located directly in the nucleus periphery that co-localize with PYL1-GFP-Emerin in XY, YZ and YZ planes, loci that do not co-localize with PYL1-GFP-Emerin, and loci that co-localize with internal PYL1-GFP-Emerin not at nuclear periphery (in rare cases). The number of loci in each category was recorded for each individual cell. Only loci of the first category that co-localize with PYL1-GFP-Emerin at the nuclear envelope were counted as nuclear periphery positioned loci. Cells containing at least one nuclear periphery positioned loci were quantified.

To determine peripheral recruitment efficacy in fixed U2OS cells (e.g., Chr7, ChrX, PTEN, CXCR4, XIST), targeted genomic loci are labeled by FISH and the nucleus are stained by DAPI. After scanning Z-stacks of confocal planes, the position of each labeled locus is viewed in 3D space to determine its position in XY, XZ and YZ planes. A genomic locus that located at the edge of nucleus (DAPI) in 3D space is categorized as a periphery-located locus. Otherwise it is considered as an internal-located locus. The number of loci in each category was recorded for each individual cell. Cells containing at least one nuclear periphery positioned loci were also quantified.

To determine the Cajal body co-localizing efficacy in fixed U2OS 2-6-3 cells, targeted LacO loci were labeled by FISH, nuclei were stained by Hoechst 33342, and Cajal bodies were labeled by PYL1-GFP-Coilin. After scanning Z-stacks of confocal planes, we identified the position of each LacO locus in 3D space. Without double counting, the loci were categorized two categories: loci that co-localize with PYL1-GFP-Coilin, and loci that do not co-localize with PYL1-GFP-Coilin. The number of loci in each category was recorded for each individual cell. Cells containing at least one Cajal body-co-localized loci were also quantified.

### Example 26. Fluorescence Assays Using Flow Cytometry Analysis

For quantification of CFP-SKL expression, U2OS 2-6-3 cells containing ABI-dCas9-P2A-mCherry and PYL1-sfGFP-Emerin or PYL1-sfGFP-Coilin were transduced with sgRNA targeting lacO loci or non-targeting sgRNAs, treated with ABA at 3 mM for 2 days and then induced with doxycycline at 50 ng/ml for 40 hours (nuclear periphery tethering) or 24 hours (Cajal body tethering). After the treatment, U2OS 2-6-3 cells were dissociated using 0.25% Trypsin EDTA (Life Technologies) and analyzed by flow cytometry on CytoFlex S (Beckman Coulter Life Sciences) using 405-nm, 488-nm and 561-nm lasers. At least 8,000 cells were analyzed for each sample. Cells were gated for positive dCas9 (mCherry) and Emerin (GFP) expression. CFP-SKL fluorescence was detected using the 405-nm laser and 450/45 filter. To quantify relative fluorescence, the average total fluorescence of untreated (without Dox and ABA) cells is set to 0, while the average total fluorescence of doxycycline induced cells (with Dox only) is set to 1. Technical replicates in 3 independent experiments are reported.

### Example 27. Real-Time RT-PCR for Endogenous Gene Expression

Real-time RT-PCR were performed to determine the expression change in PPP1R2 and ACAP2 gene adjacent to targeted Chr3 loci after genomic re-organization. For each sample, total RNAs were isolated using RNeasy Plus Mini Kit (Qiagen Cat 74134) and cDNAs were synthesized using the iScript cDNA Synthesis Kit (BioRad, Cat 1708890), according to manufacturer's protocols. Quantitative PCR was performed using the PrimePCR assay with the SYBR Green Master Mix (BioRad), and run on Biorad CFX384 real-time system (C1000 Touch Thermal Cycler), according to manufacturer's instructions. Cq values was used to quantify gene expression. The relative expression of the PPP1R2 and ACAP2 genes was normalized to GAPDH control. To calculate the relative mRNA expression level, the relative expression of each treatment was normalized by setting the average value in non-ABA treated samples as 1. Replicates in 3 experiments are reported.

### Example 28. Cell Viability Assay

Cell viability assay was performed using Alamar blue cell viability reagents (ThermoFisher Scientific), which measures the metabolic activity of the cells. For each condition, 100 μl cells treated with and without ABA were seeded at equal concentration (500-1000 cells/well) in the same 96-well plate. At the time of detection, 10 μl of Alamar blue reagents were added to each well and the plates were incubated at 37° C. for 1 hour. After that, the fluorescent intensity was measured in the Synergy H1 microplate reader (Biotek Inc.) using the excitation wavelength at 540 nm and the emission wavelength at 585 nm. Average fluorescent intensity of wells containing only 100 μl culture medium (with and without ABA) was used as blanks. For each well, the relative fluorescent intensity is calculated by subtracting background (average intensity of blank wells) from its raw fluorescent intensity. To calculate the relative cell viability, the relative florescent intensity in each well was normalized by setting the average value in non-ABA treated wells as 1. Replicates in 3 experiments are reported.

### Example 29. Cell Cycle Cytometry Analysis

To quantify how telomere nuclear periphery tethering affect cell cycle progression, U2OS cells containing nuclear periphery tethering system were treated with lentivirus mixtures coding sgTelomere and TRF1-mcherry, or lentivirus coding a non-targeting sgRNA. Telomere tethering was confirmed by microscopy after 2 days of ABA treatment. After 3 day of ABA treatment, control and treated cells were dissociated using 0.25% Trypsin EDTA, with stained Hoechst 33342 at 1:1000 dilution for 1 h, and analyzed by flow cytometry on CytoFlex S (Beckman Coulter Life Sciences) using 405-nm lasers. At least 20,000 cells were analyzed for each sample. Cell cycle analysis was performed using FlowJO.

### Example 30. Identification of Human Repetitive Sequence Clusters

The software Tandem Repeats Finder (Benson, 1999) was used to identify all tandem repeats of 14-nucleotides or longer sequences from the human genome (hg38). Regions that contain ten or more identical tandem repeats were defined a “repetitive sequence cluster.” These repetitive sequence clusters were to each human chromosome. Distances between the repetitive sequence clusters and genes were calculated using the BEDTools suite.

### Example 31. Short-Term Dynamic Tracking of Endogenous Loci

Genomic loci tracking was performed using the TrackMate plugin (Tinevez et al., 2017) in Fiji. For tracking genomic loci, the estimated blob diameter was set between 0.5-1 μm. Linking max distance was set to 2 μm, and gap closing distance was set to 3 μm and gap closing max frame was set to 2. Position of each locus (x^{t}, y^{t}) at different time point (t) were measured, analyzed in Excel and plotted in GraphPad Prism 7. The movement step (dx, dy) was calculated by subtracting the position of a previous time point from the new position: dx^{t}=x^{t}−x^{t-1} & dy^{t}=y^{t}−y^{t-1}, where (x^{t}, y^{t}) is position of a locus at time t, while (x^{t-1}, y^{t-1}) is the position of the locus at the previous time point (t−1). Step distance=\sqrt{(x^{t}−x^{t-1})²+(y^{t}−y^{t-1})²} is calculated as how far a locus move away from its position at the previous time point.

To compare step distances, 1696 step distances of 19 interior-localized Chr3 loci and 1669 step distances of 14 periphery-localized Chr3 loci were analyzed. The two-side t-test with unequal variance was performed. Histogram were analyzed using Histogram function in Excel and plotted in in GraphPad Prism 7.

### Example 32. Statistics Analysis

For quantification of re-localization efficacy (FIGS. 8, 9, 16, 18-21, 30, 33, 36, 38, and 39), p value was calculated using Fisher's exact test in GraphPad, and error bars show standard error of the mean (SEMs) calculated according to Bernoulli distributions. The numbers of counted loci/cells are listed at the bottom of each figure. For FIGS. 26, 42, 43, 46-51, 53, 56, and 57, p value was calculated using two-sided t-test with unequal variance in Excel and error bars show standard deviations. Duplicates in 3 experiments were analyzed. For FIG. 27, fit Gamma distributions were fit by maximum likelihood using the R package fitdistrplus and tested the goodness of fit using the Kolmogorov-Smirnov test (p=0.06 for periphery loci & p=0.77 for interior loci).

### V. References

Ballister, E. R., Aonbangkhen, C., Mayo, A. M., Lampson, M. A., and Chenoweth, D. M. (2014). Localized light-induced protein dimerization in living cells using a photocaged dimerizer. Nat Commun 5, 5475.
Barrangou, R., Fremaux, C., Deveau, H., Richards, M., Boyaval, P., Moineau, S., Romero, D. A., and Horvath, P. (2007). CRISPR provides acquired resistance against viruses in prokaryotes. Science 315, 1709-1712.
Benson, G. (1999). Tandem repeats finder: a program to analyze DNA sequences. Nucleic Acids Res 27, 573-580.
Berk, J. M., Tifft, K. E., and Wilson, K. L. (2013). The nuclear envelope LEM-domain protein emerin. Nucleus 4, 298-314.
Bernardi, R., and Pandolfi, P. P. (2003). Role of PML and the PML-nuclear body in the control of programmed cell death. Oncogene 22, 9048-9057.
Bickmore, W. A. (2013). The spatial organization of the human genome. Annu Rev Genomics Hum Genet 14, 67-84.
Bonev, B., and Cavalli, G. (2016). Organization and function of the 3D genome. Nat Rev Genet 17, 772.
Chen, B., Gilbert, L. A., Cimini, B. A., Schnitzbauer, J., Zhang, W., Li, G. W., Park, J., Blackburn, E. H., Weissman, J. S., Qi, L. S., et al. (2013). Dynamic imaging of genomic loci in living human cells by an optimized CRISPR/Cas system. Cell 155, 1479-1491.
Cheutin, T., McNairn, A. J., Jenuwein, T., Gilbert, D. M., Singh, P. B., and Misteli, T. (2003). Maintenance of stable heterochromatin domains by dynamic HP1 binding. Science 299, 721-725.
Clowney, E. J., LeGros, M. A., Mosley, C. P., Clowney, F. G., Markenskoff-Papadimitriou, E. C., Myllys, M., Barnea, G., Larabell, C. A., and Lomvardas, S. (2012). Nuclear aggregation of olfactory receptor genes governs their monogenic expression. Cell 151, 724-737.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
Crabbe, L., Cesare, A. J., Kasuboski, J. M., Fitzpatrick, J. A., and Karlseder, J. (2012). Human telomeres are tethered to the nuclear envelope during postmitotic nuclear assembly. Cell Rep 2, 1521-1529.
Cristofari, G., Adolf, E., Reichenbach, P., Sikora, K., Terns, R. M., Terns, M. P., and Lingner, J. (2007). Human telomerase RNA accumulation in Cajal bodies facilitates telomerase recruitment to telomeres and telomere elongation. Mol Cell 27, 882-889.
de Koning, A. P., Gu, W., Castoe, T. A., Batzer, M. A., and Pollock, D. D. (2011). Repetitive elements may comprise over two-thirds of the human genome. PLoS Genet 7, e1002384.
Dekker, J., Marti-Renom, M. A., and Mirny, L. A. (2013). Exploring the three-dimensional organization of genomes: interpreting chromatin interaction data. Nat Rev Genet 14, 390-403.
Dekker, J., Rippe, K., Dekker, M., and Kleckner, N. (2002). Capturing chromosome conformation. Science 295, 1306-1311.
Denker, A., and de Laat, W. (2016). The second decade of 3C technologies: detailed insights into nuclear organization. Genes Dev 30, 1357-1382.
Finlan, L. E., Sproul, D., Thomson, I., Boyle, S., Kerr, E., Perry, P., Ylstra, B., Chubb, J. R., and Bickmore, W. A. (2008). Recruitment to the nuclear periphery can alter expression of genes in human cells. PLoS Genet 4, e1000039.
Gall, J. G. (2000). Cajal bodies: the first 100 years. Annu Rev Cell Dev Biol 16, 273-300.
Gao, Y., Xiong, X., Wong, S., Charles, E. J., Lim, W. A., and Qi, L. S. (2016). Complex transcriptional modulation with orthogonal and inducible dCas9 regulators. Nat Methods 13, 1043-1049.
Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661.
Grimm, J. B., English, B. P., Chen, J., Slaughter, J. P., Zhang, Z., Revyakin, A., Patel, R., Macklin, J. J., Normanno, D., Singer, R. H., et al. (2015). A general method to improve fluorophores for live-cell and single-molecule microscopy. Nat Methods 12, 244-250, 243 p following 250.
Gross, S. P., Vershinin, M., and Shubeita, G. T. (2007). Cargo transport: two motors are sometimes better than one. Curr Biol 17, R478-486.
Guan, J., Liu, H., Shi, X., Feng, S., and Huang, B. (2017). Tracking Multiple Genomic Elements Using Correlative CRISPR Imaging and Sequential DNA FISH. Biophys J 112, 1077-1084.
Hilton, I. B., D'Ippolito, A. M., Vockley, C. M., Thakore, P. I., Crawford, G. E., Reddy, T. E., and Gersbach, C. A. (2015). Epigenome editing by a CRISPR-Cas9-based acetyltransferase activates genes from promoters and enhancers. Nat Biotechnol 33, 510-517.
Hoogenraad, C. C., Wulf, P., Schiefermeier, N., Stepanova, T., Galjart, N., Small, J. V., Grosveld, F., de Zeeuw, C. I., and Akhmanova, A. (2003). Bicaudal D induces selective dynein-mediated microtubule minus end-directed transport. The EMBO Journal 22, 6004-6015.
Jady, B. E., Richard, P., Bertrand, E., and Kiss, T. (2006). Cell cycle-dependent recruitment of telomerase RNA and Cajal bodies to human telomeres. Mol Biol Cell 17, 944-954.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Jinek, M., East, A., Cheng, A., Lin, S., Ma, E., and Doudna, J. (2013). RNA-programmed genome editing in human cells. Elife 2, e00471.
Kaiser, T. E., Intine, R. V., and Dundr, M. (2008). De novo formation of a subnuclear body. Science 322, 1713-1717.
Kapitein, L. C., Schlager, M. A., van der Zwan, W. A., Wulf, P. S., Keijzer, N., and Hoogenraad, C. C. (2010). Probing intracellular motor protein activity using an inducible cargo trafficking assay. Biophysical Journal 99, 2143-2152.
Kearns, N. A., Pham, H., Tabak, B., Genga, R. M., Silverstein, N. J., Garber, M., and Maehr, R. (2015). Functional annotation of native enhancers with a Cas9-histone demethylase fusion. Nat Methods 12, 401-403.
Knight, S. C., Xie, L., Deng, W., Guglielmi, B., Witkowsky, L. B., Bosanac, L., Zhang, E. T., El Beheiry, M., Masson, J. B., Dahan, M., et al. (2015). Dynamics of CRISPR-Cas9 genome interrogation in living cells. Science 350, 823-826.
Komor, A. C., Kim, Y. B., Packer, M. S., Zuris, J. A., and Liu, D. R. (2016). Programmable editing of a target base in genomic DNA without double-stranded DNA cleavage. Nature 533, 420-424.
Kosak, S. T., Skok, J. A., Medina, K. L., Riblet, R., Le Beau, M. M., Fisher, A. G., and Singh, H. (2002). Subnuclear compartmentalization of immunoglobulin loci during lymphocyte development. Science 296, 158-162.
Kumaran, R. I., and Spector, D. L. (2008). A genetic locus targeted to the nuclear periphery in living cells maintains its transcriptional competence. J Cell Biol 180, 51-65.
Langer-Safer, P. R., Levine, M., and Ward, D. C. (1982). Immunological method for mapping genes on Drosophila polytene chromosomes. Proc Natl Acad Sci USA 79, 4381-4385.
Levine, M., Cattoglio, C., and Tjian, R. (2014). Looping back to leap forward: transcription enters a new era. Cell 157, 13-25.
Liang, F. S., Ho, W. Q., and Crabtree, G. R. (2011). Engineering the ABA plant stress pathway for regulation of induced proximity. Sci Signal 4, rs2.
Ma, H., Tu, L. C., Naseri, A., Huisman, M., Zhang, S., Grunwald, D., and Pederson, T. (2016). Multiplexed labeling of genomic loci with dCas9 and engineered sgRNAs using CRISPRainbow. Nat Biotechnol 34, 528-530.
Machyna, M., Neugebauer, K. M., and Stanek, D. (2015). Coilin: The first 25 years. RNA Biol 12, 590-596.
Mali, P., Yang, L., Esvelt, K. M., Aach, J., Guell, M., DiCarlo, J. E., Norville, J. E., and Church, G. M. (2013). RNA-guided human genome engineering via Cas9. Science 339, 823-826.
Mao, Y. S., Zhang, B., and Spector, D. L. (2011). Biogenesis and function of nuclear bodies. Trends Genet 27, 295-306.
McCaffrey, M. W., and Lindsay, A. J. (2012). Roles for myosin Va in RNA transport and turnover. Biochem Soc Trans 40, 1416-1420.
Morgan, S. L., Mariano, N. C., Bermudez, A., Arruda, N. L., Wu, F., Luo, Y., Shankar, G., Jia, L., Chen, H., Hu, J. F., et al. (2017). Manipulation of nuclear architecture through CRISPR-mediated chromosomal looping. Nat Commun 8, 15993.
Neugebauer, K. M. (2017). Special focus on the Cajal Body. RNA Biol 14, 669-670.
Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
Reddy, K. L., Zullo, J. M., Bertolino, E., and Singh, H. (2008). Transcriptional repression mediated by repositioning of genes to the nuclear lamina. Nature 452, 243-247.
Schmitt, A. D., Hu, M., and Ren, B. (2016). Genome-wide mapping and analysis of chromosome architecture. Nat Rev Mol Cell Biol 17, 743-755.
Schreer, A., Tinson, C., Sherry, J. P., and Schirmer, K. (2005). Application of Alamar blue/5-carboxyfluorescein diacetate acetoxymethyl ester as a noninvasive cell viability assay in primary hepatocytes from rainbow trout. Anal Biochem 344, 76-85.
Shachar, S., Voss, T. C., Pegoraro, G., Sciascia, N., and Misteli, T. (2015). Identification of Gene Positioning Factors Using High-Throughput Imaging Mapping. Cell 162, 911-923.
Shevtsov, S. P., and Dundr, M. (2011). Nucleation of nuclear bodies by RNA. Nat Cell Biol 13, 167-173.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Smogorzewska, A., and de Lange, T. (2002). Different telomere damage signaling pathways in human and mouse cells. EMBO J 21, 4338-4348.
Smoyer, C. J., and Jaspersen, S. L. (2014). Breaking down the wall: the nuclear envelope during mitosis. Curr Opin Cell Biol 26, 1-9.
Takei, Y., Shah, S., Harvey, S., Qi, L. S., and Cai, L. (2017). Multiplexed Dynamic Imaging of Genomic Loci by Combined CRISPR Imaging and DNA Sequential FISH. Biophys J 112, 1773-1776.
Tanenbaum, M. E., Gilbert, L. A., Qi, L. S., Weissman, J. S., and Vale, R. D. (2014). A protein-tagging system for signal amplification in gene expression and fluorescence imaging. Cell 159, 635-646.
Tinevez, J. Y., Perry, N., Schindelin, J., Hoopes, G. M., Reynolds, G. D., Laplantine, E., Bednarek, S. Y., Shorte, S. L., and Eliceiri, K. W. (2017). TrackMate: An open and extensible platform for single-particle tracking. Methods 115, 80-90.
Tsuchiya, Y., Hase, A., Ogawa, M., Yorifuji, H., and Arahata, K. (1999). Distinct regions specify the nuclear membrane targeting of emerin, the responsible protein for Emery-Dreifuss muscular dystrophy. Eur J Biochem 259, 859-865.
van Steensel, B., and Belmont, A. S. (2017). Lamina-Associated Domains: Links with Chromosome Architecture, Heterochromatin, and Gene Repression. Cell 169, 780-791.
Vernier, M., Bourdeau, V., Gaumont-Leclerc, M. F., Moiseeva, O., Begin, V., Saad, F., Mes-Masson, A. M., and Ferbeyre, G. (2011). Regulation of E2Fs and senescence by PML nuclear bodies. Genes Dev 25, 41-50.
Wang, Q., Sawyer, I. A., Sung, M. H., Sturgill, D., Shevtsov, S. P., Pegoraro, G., Hakim, O., Baek, S., Hager, G. L., and Dundr, M. (2016). Cajal bodies are linked to genome conformation. Nat Commun 7, 10966.
Williams, R. R., Azuara, V., Perry, P., Sauer, S., Dvorkina, M., Jorgensen, H., Roix, J., McQueen, P., Misteli, T., Merkenschlager, M., et al. (2006). Neural induction promotes large-scale chromatin reorganisation of the Mashl locus. J Cell Sci 119, 132-140.
Yu, M., and Ren, B. (2017). The Three-Dimensional Organization of Mammalian Genomes. Annu Rev Cell Dev Biol 33, 265-289.
Zhu, L., and Brangwynne, C. P. (2015). Nuclear bodies: the emerging biophysics of nucleoplasmic phases. Curr Opin Cell Biol 34, 23-30.
Zuleger, N., Kelly, D. A., Richardson, A. C., Kerr, A. R., Goldberg, M. W., Goryachev, A. B., and Schirmer, E. C. (2011). System analysis shows distinct mechanisms and common principles of nuclear envelope protein dynamics. J Cell Biol 193, 109-123.
Zullo, J. M., Demarco, I. A., Pique-Regi, R., Gaffney, D. J., Epstein, C. B., Spooner, C. J., Luperchio, T. R., Bernstein, B. E., Pritchard, J. K., Reddy, K. L., et al. (2012). DNA sequence-dependent compartmentalization and silencing of chromatin at the nuclear lamina. Cell 149, 1474-1487.

### VI. Exemplary Embodiments

Exemplary embodiments provided in accordance with the presently disclosed subject matter include, but are not limited to, the claims and the following embodiments:

1. A system for controlling the spatial and temporal positioning of a target polynucleotide in a compartment of a cell, the system comprising:

(a) a compartment-specific protein linked to a first dimerization domain; and

(b) an actuator moiety that targets the target polynucleotide, wherein the actuator moiety is linked to a second dimerization domain that is capable of assembling into a dimer with the first dimerization domain.

2. The system of embodiment 1, wherein the target polynucleotide comprises genomic DNA.

3. The system of embodiment 1, wherein the target polynucleotide comprises RNA.

4. The system of any one of embodiments 1-3, wherein the actuator moiety comprises a Cas protein, and wherein the system further comprises:

(c) a guide RNA that complexes with the actuator moiety and hybridizes to the target polynucleotide.

5. The system of any one of embodiments 1-3, wherein the actuator moiety comprises an RNA-binding protein, wherein the system further comprises:

(c) a guide RNA that complexes with the actuator moiety and hybridizes to the target polynucleotide, and wherein the system optionally further comprises:

(d) a Cas protein that complexes with the guide RNA.

6. The system of embodiment 4 or 5, wherein the Cas protein substantially lacks DNA cleavage activity.

7. The system of any one of embodiments 4-6, wherein the Cas protein is a Cas9 protein, a Cas12 protein, a Cas13 protein, a CasX protein, or a CasY protein.

8. The system of embodiment 7, wherein the Cas12 protein is selected from the group consisting of Cas12a, Cas12b, Cas12c, Cas12d, and Cas12e.

9. The system of embodiment 7, wherein the Cas13 protein is selected from the group consisting of Cas13a, Cas13b, Cas13c, and Cas13d.

10. The system of embodiment 5, wherein the RNA-binding protein is ADAR1 or ADAR2 and the guide RNA comprises an ADAR-recruiting RNA (arRNA).

11. The system of any one of embodiments 1-3, wherein the actuator moiety comprises a binding protein that hybridizes to the target polynucleotide, wherein the binding protein is a zinc finger nuclease or a TALE nuclease.

12. The system of any one of embodiments 1-3, wherein the actuator moiety comprises an Argonaute protein complexed with a guide polynucleotide, wherein the guide polynucleotide is a guide RNA or a guide DNA, and wherein the guide polynucleotide hybridizes to the target polynucleotide.

13. The system of any one of embodiments 1-12, wherein the compartment-specific protein is selected from the group consisting of a protein endogenous to the compartment, a regulator protein, a motor protein, a DNA repair protein, and a combination thereof.

14. The system of any one of embodiments 1-13, wherein the compartment is a nuclear compartment.

15. The system of embodiment 14, wherein the nuclear compartment comprises an inner nuclear membrane.

16. The system of embodiment 15, wherein the compartment-specific protein comprises Emerin, Lap2beta, Lamin B, or a combination thereof.

17. The system of embodiment 14, wherein the nuclear compartment comprises a Cajal body.

18. The system of embodiment 17, wherein the compartment-specific protein comprises coilin, SMN, Gemin 3, SmD1, SmE, or a combination thereof.

19. The system of embodiment 14, wherein the nuclear compartment comprises a nuclear speckle.

20. The system of embodiment 19, wherein the compartment-specific protein comprises SC35.

21. The system of embodiment 14, wherein the nuclear compartment comprises a PML body.

22. The system of embodiment 21, wherein the compartment-specific protein comprises PML, SP100, or a combination thereof.

23. The system of embodiment 14, wherein the nuclear compartment comprises a nuclear core complex.

24. The system of embodiment 23, wherein the compartment-specific protein comprises Nup50, Nup98, Nup53, Nup153, Nup62, or a combination thereof.

25. The system of embodiment 14, wherein the nuclear compartment comprises a nucleolus.

26. The system of embodiment 25, wherein the compartment-specific protein comprises nucleolar protein B23.

27. The system of embodiment 14, wherein the nuclear compartment comprises heterochromatin.

28. The system of embodiment 27, wherein the compartment-specific protein comprises HP1, KRAB-ZFP, a truncated form thereof, or a combination thereof.

29. The system of embodiment 14, wherein the nuclear compartment comprises a nuclear body.

30. The system of embodiment 29, wherein the compartment-specific protein comprises 53BP1, Rad51, or a combination thereof.

31. The system of any one of embodiments 1-13, wherein the compartment is a cytoplasmic compartment.

32. The system of embodiment 31, wherein the cytoplasmic compartment comprises a cytoskeletal component.

33. The system of embodiment 32, wherein the compartment-specific protein comprises a kinesin, dynein, myosin, or a combination thereof.

34. The system of any one of embodiments 1-33, wherein the compartment-specific protein is further linked to a fluorescent protein.

35. The system of any one of embodiments 1-34, wherein the actuator moiety is further linked to a fluorescent protein.

36. The system of any one of embodiments 1-35, wherein the first dimerization domain and the second dimerization domain assemble to form a dimer only in the presence of a ligand, light, or an enzyme.

37. The system of embodiment 36, wherein the first dimerization domain and the second dimerization domain each bind to the ligand in the presence of the ligand.

38. The system of embodiment 36 or 37, wherein the ligand is a chemical inducer or an optogenetic inducer.

39. A method of controlling the spatial and temporal positioning of a target polynucleotide in a compartment of a cell, the method comprising:

(a) providing a compartment-specific protein linked to a first dimerization domain;

(b) providing an actuator moiety linked to a second dimerization domain;

(c) forming a complex comprising the actuator moiety and the target polynucleotide; and

(d) assembling a dimer comprising the first dimerization domain and the second dimerization domain, thereby positioning the target polynucleotide in the compartment.

40. The method of embodiment 39, wherein the target polynucleotide is not endogenous to the compartment.

41. The method of embodiment 39 or 40, wherein the positioning of the target polynucleotide comprises regulating the expression of the target polynucleotide.

42. The method of embodiment 41, wherein the regulating comprises decreasing the expression of the target polynucleotide.

43. The method of embodiment 41, wherein the regulating comprises increasing the expression of the target polynucleotide.

44. The method of any one of embodiments 39-43, wherein the positioning of the target polynucleotide further comprises regulating the expression of one or more additional polynucleotides endogenous to the compartment.

45. The method of any one of embodiments 39-44, wherein the positioning of the target polynucleotide comprises altering cellular function, cell fate, cell growth, apoptosis, and/or cell differentiation.

46. The method of embodiment 45, wherein the target polynucleotide comprises a telomere.

47. The method of any one of embodiments 39-46, wherein the positioning of the target polynucleotide further comprises creating one or more additional compartments within the cell.

48. The method of any one of embodiments 39-47, wherein the positioning of the target polynucleotide further comprises repairing a DNA break.

49. The method of embodiment 48, wherein the repairing comprises introducing exogenous DNA.

50. The method of embodiment 49, wherein the introducing comprises recombination, non-homologous end-joining, or homology-directed repair.

51. The method of any one of embodiments 39-50, wherein the positioning of the target polynucleotide induces a phase separation to form the compartment.

52. The method of embodiment 51, wherein the compartment is an artificial aggregate comprising protein, RNA, DNA, or a combination thereof.

53. The method of embodiment 51 or 52, wherein the compartment is a nuclear body or a cellular body.

54. The method of any one of embodiments 39-53, wherein the positioning of the target polynucleotide induces the formation of a nuclear body that facilitates DNA repair and improves gene editing efficiency.

55. The method of any one of embodiments 39-54, wherein the target polynucleotide comprises genomic DNA.

56. The method of any one of embodiments 39-54, wherein the target polynucleotide comprises RNA.

57. The method of any one of embodiments 39-56, wherein the actuator moiety comprises a Cas protein, and wherein the method further comprises:

(c) providing a guide RNA that complexes with the actuator moiety and hybridizes to the target polynucleotide.

58. The method of any one of embodiments 39-56, wherein the actuator moiety comprises an RNA-binding protein, wherein the method further comprises:

(c) providing a guide RNA that complexes with the actuator moiety and hybridizes to the target polynucleotide, and wherein the method optionally further comprises:

(d) providing a Cas protein that complexes with the guide RNA.

59. The method of embodiment 57 or 58, wherein the Cas protein substantially lacks DNA cleavage activity.

60. The method of any one of embodiments 57-59, wherein the Cas protein is a Cas9 protein, a Cas12 protein, a Cas13 protein, a CasX protein, or a CasY protein.

61. The method of embodiment 60, wherein the Cas12 protein is selected from the group consisting of Cas12a, Cas12b, Cas12c, Cas12d, and Cas12e.

62. The method of embodiment 60, wherein the Cas13 protein is selected from the group consisting of Cas13a, Cas13b, Cas13c, and Cas13d.

63. The method of embodiment 58, wherein the RNA-binding protein is ADAR1 or ADAR2 and the guide RNA comprises an ADAR-recruiting RNA (arRNA).

64. The method of any one of embodiments 39-56, wherein the actuator moiety comprises a binding protein that hybridizes to the target polynucleotide, wherein the binding protein is a zinc finger nuclease or a TALE nuclease.

65. The method of any one of embodiments 39-56, wherein the actuator moiety comprises an Argonaute protein complexed with a guide polynucleotide, wherein the guide polynucleotide is a guide RNA or a guide DNA, and wherein the guide polynucleotide hybridizes to the target polynucleotide.

66. The method of any one of embodiments 39-65, wherein the compartment-specific protein is selected from the group consisting of a protein endogenous to the compartment, a regulator protein, a motor protein, a DNA repair protein, and a combination thereof.

67. The method of any one of embodiments 39-66, wherein the compartment is a nuclear compartment.

68. The method of embodiment 67, wherein the nuclear compartment comprises an inner nuclear membrane.

69. The method of embodiment 68, wherein the compartment-specific protein comprises Emerin, Lap2beta, Lamin B, or a combination thereof.

70. The method of embodiment 67, wherein the nuclear compartment comprises a Cajal body.

71. The method of embodiment 70, wherein the compartment-specific protein comprises coilin, SMN, Gemin 3, SmD1, SmE, or a combination thereof.

72. The method of embodiment 67, wherein the nuclear compartment comprises a nuclear speckle.

73. The method of embodiment 72, wherein the compartment-specific protein comprises SC35.

74. The method of embodiment 67, wherein the nuclear compartment comprises a PML body.

75. The method of embodiment 74, wherein the compartment-specific protein comprises PML, SP100, or a combination thereof.

76. The method of embodiment 67, wherein the nuclear compartment comprises a nuclear core complex.

77. The method of embodiment 76, wherein the compartment-specific protein comprises Nup50, Nup98, Nup53, Nup153, Nup62, or a combination thereof.

78. The method of embodiment 67, wherein the nuclear compartment comprises a nucleolus.

79. The method of embodiment 78, wherein the compartment-specific protein comprises nucleolar protein B23.

80. The method of embodiment 67, wherein the nuclear compartment comprises heterochromatin.

81. The method of embodiment 80, wherein the compartment-specific protein comprises HP1, KRAB-ZFP, a truncated form thereof, or a combination thereof.

82. The method of embodiment 67, wherein the nuclear compartment comprises a nuclear body.

83. The method of embodiment 82, wherein the compartment-specific protein comprises 53BP1, Rad51, or a combination thereof.

84. The method of any one of embodiments 39-66, wherein the compartment is a cytoplasmic compartment.

85. The method of embodiment 84, wherein the cytoplasmic compartment comprises a cytoskeletal component.

86. The method of embodiment 85, wherein the compartment-specific protein comprises a kinesin, dynein, myosin, or a combination thereof.

87. The method of any one of embodiments 39-86, wherein the compartment-specific protein is further linked to a fluorescent protein.

88. The method of any one of embodiments 39-87, wherein the actuator moiety is further linked to a fluorescent protein.

89. The method of any one of embodiments 39-88, wherein the assembling occurs only in the presence of a ligand, light, or an enzyme.

90. The method of embodiment 89, wherein the first dimerization domain and the second dimerization domain each bind to the ligand in the presence of the ligand.

91. The method of embodiment 89 or 90, wherein the ligand is a chemical inducer or an optogenetic inducer.

Although the foregoing invention has been described in some detail by way of illustration and example for purpose of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1-118. (canceled)

119. A system for controlling positioning of a target polynucleotide in a compartment of a cell, the system comprising:
a compartmentalization moiety linked to a first coupling domain; and
an actuator moiety for forming a complex comprising the actuator moiety and the target polynucleotide, wherein the actuator moiety is linked to a second coupling domain that is capable of forming an assembly with the first coupling domain,
wherein formation of the complex and the assembly effects the positioning of the target polynucleotide towards the compartment of the cell.

120. The system of claim 119, wherein the target polynucleotide is not endogenous to the compartment.

121. The system of claim 119, wherein the compartment is an artificial aggregate.

122. The system of claim 119, wherein the compartment is a nuclear compartment or a cytoplasmic compartment.

123. The system of claim 122, wherein the compartment is the nuclear compartment selected from the group consisting of an inner nuclear membrane, a Cajal body, a nuclear speckle, a Promyelocytic leukemia protein body, a nuclear core complex, a nucleolus, a heterochromatin, and a nuclear body.

124. The system of claim 122, wherein the compartment is the cytoplasmic compartment comprising a cytoskeletal component.

125. The system of claim 119, wherein formation of the assembly is induced by a ligand, light, or an enzyme.

126. The system of claim 125, wherein the first coupling domain and the second coupling domain both bind to the ligand.

127. The system of claim 119, wherein the target polynucleotide comprises a plurality of repetitive sequences.

128. The system of claim 119, wherein the target polynucleotide is endogenous to the cell.

129. The system of claim 119, wherein the target polynucleotide comprises a DNA sequence or a RNA sequence.

130. The system of claim 119, wherein the target polynucleotide comprises a telomere.

131. The system of claim 119, wherein the actuator moiety comprises an exogenous nuclease.

132. The system of claim 119, wherein the actuator moiety is a Cas protein that is directed to the target polynucleotide by a guide nucleic acid molecule, to form the complex.

133. The system of claim 119, wherein the positioning of the target polynucleotide to the compartment effects a change in expression level of the target polynucleotide.

134. The system of claim 119, wherein the positioning of the target polynucleotide to the compartment induces epigenetic modification of the target polynucleotide.

135. The system of claim 119, wherein the positioning of the target polynucleotide to the compartment in the cell is for regulating function of the cell, fate of the cell, growth of the cell, apoptosis of the cell, or differentiation of the cell.

136. The system of claim 119, wherein the positioning of the target polynucleotide to the compartment in the cell effects DNA repair or enhanced gene editing efficiency.

137. A method for controlling positioning of a target polynucleotide in a compartment of a cell, the method comprising:
(a) contacting the cell with (i) a compartmentalization moiety linked to a first coupling domain, and (ii) an actuator moiety for forming a complex comprising the actuator moiety and the target polynucleotide, wherein the actuator moiety is linked to a second coupling domain that is capable of forming an assembly with the first coupling domain;
(b) forming the complex and the assembly, to effect the positioning of the target polynucleotide towards the compartment of the cell.

138. The method of claim 136, wherein the forming the assembly comprises subjecting the cell to a ligand, light, or an enzyme.


SEQUENCE LISTING (41 sequences)

SEQ ID NO: 1 - protein, 25 aa, Artificial Sequence
  Description of Artificial Sequence Synthetic peptide
  MISC_FEATURE (1)..(25): This sequence may encompass 2-5 “Gly Gly Gly Gly Ser” repeating units
  See specification as filed for detailed description of substitutions and preferred embodiments
GGGGSGGGGSGGGGSGGGGSGGGGS

SEQ ID NO: 2 - protein, 5 aa, Artificial Sequence
  Description of Artificial Sequence Synthetic peptide
GGGGS

SEQ ID NO: 3 - DNA, 11 bp, Artificial Sequence
  Description of Artificial Sequence Synthetic oligonucleotide
tgatatcacag

SEQ ID NO: 4 - DNA, 11 bp, Artificial Sequence
  Description of Artificial Sequence Synthetic oligonucleotide
accattccttc

SEQ ID NO: 5 - DNA, 21 bp, Artificial Sequence
  Description of Artificial Sequence Synthetic oligonucleotide
ggcaaggcaaggcaaggcaca

SEQ ID NO: 6 - DNA, 19 bp, Artificial Sequence
  Description of Artificial Sequence Synthetic oligonucleotide
gctcttatggtgagagtgt

SEQ ID NO: 7 - DNA, 18 bp, Artificial Sequence
  Description of Artificial Sequence Synthetic oligonucleotide
gttagggttagggttagg

SEQ ID NO: 8 - DNA, 17 bp, Artificial Sequence
  Description of Artificial Sequence Synthetic oligonucleotide
gctcacaattccacatg

SEQ ID NO: 9 - DNA, 20 bp, Artificial Sequence
  Description of Artificial Sequence Synthetic oligonucleotide
gccacatgtggaattgtgag

SEQ ID NO: 10 - DNA, 32 bp, Artificial Sequence
  Description of Artificial Sequence Synthetic oligonucleotide
gtacgttctctatcactgatatgatatcacag

SEQ ID NO: 11 - DNA, 21 bp, Artificial Sequence
  Description of Artificial Sequence Synthetic oligonucleotide
gatcagctctctcacggtgac

SEQ ID NO: 12 - DNA, 21 bp, Artificial Sequence
  Description of Artificial Sequence Synthetic oligonucleotide
gcacttggctgagtccacagt

SEQ ID NO: 13 - DNA, 21 bp, Artificial Sequence
  Description of Artificial Sequence Synthetic oligonucleotide
gacatcggagaatgcacgctc

SEQ ID NO: 14 - DNA, 20 bp, Artificial Sequence
  Description of Artificial Sequence Synthetic oligonucleotide
gaataggtcgatgtagagca

SEQ ID NO: 15 - DNA, 21 bp, Artificial Sequence
  Description of Artificial Sequence Synthetic oligonucleotide
gccgcgttctgtaagaatcgg

SEQ ID NO: 16 - DNA, 20 bp, Artificial Sequence
  Description of Artificial Sequence Synthetic oligonucleotide
gtaagtcctatgacagaagc

SEQ ID NO: 17 - DNA, 21 bp, Artificial Sequence
  Description of Artificial Sequence Synthetic oligonucleotide
gagatatactgttagcgcctt

SEQ ID NO: 18 - DNA, 20 bp, Artificial Sequence
  Description of Artificial Sequence Synthetic oligonucleotide
ggctgtagacatcaatgctt

SEQ ID NO: 19 - DNA, 21 bp, Artificial Sequence
  Description of Artificial Sequence Synthetic oligonucleotide
gatcattgcaggtaagaagtg

SEQ ID NO: 20 - DNA, 21 bp, Artificial Sequence
  Description of Artificial Sequence Synthetic oligonucleotide
gctccaacactctaccttgta

SEQ ID NO: 21 - DNA, 20 bp, Artificial Sequence
  Description of Artificial Sequence Synthetic oligonucleotide
ggaagtgcttacgagtcaat

SEQ ID NO: 22 - DNA, 21 bp, Artificial Sequence
  Description of Artificial Sequence Synthetic oligonucleotide
gctctgtcagtaactgataag

SEQ ID NO: 23 - DNA, 20 bp, Artificial Sequence
  Description of Artificial Sequence Synthetic oligonucleotide
gcaagcttacctgatagatt

SEQ ID NO: 24 - DNA, 21 bp, Artificial Sequence
  Description of Artificial Sequence Synthetic oligonucleotide
gttccatcttctaagtgtcct

SEQ ID NO: 25 - DNA, 20 bp, Artificial Sequence
  Description of Artificial Sequence Synthetic oligonucleotide
gaactgagacttgtgacaca

SEQ ID NO: 26 - DNA, 20 bp, Artificial Sequence
  Description of Artificial Sequence Synthetic oligonucleotide
gagttagaagtcttaagacc

SEQ ID NO: 27 - DNA, 21 bp, Artificial Sequence
  Description of Artificial Sequence Synthetic oligonucleotide
gttccatccaactcaggcctt

SEQ ID NO: 28 - DNA, 21 bp, Artificial Sequence
  Description of Artificial Sequence Synthetic oligonucleotide
gcctgaggcttctatctatct

SEQ ID NO: 29 - DNA, 20 bp, Artificial Sequence
  Description of Artificial Sequence Synthetic oligonucleotide
ggaaggctcatatggataga

SEQ ID NO: 30 - DNA, 20 bp, Artificial Sequence
  Description of Artificial Sequence Synthetic oligonucleotide
gatcatctcacatggcagcc

SEQ ID NO: 31 - DNA, 20 bp, Artificial Sequence
  Description of Artificial Sequence Synthetic oligonucleotide
ggctatcagagcaagcattg

SEQ ID NO: 32 - DNA, 20 bp, Artificial Sequence
  Description of Artificial Sequence Synthetic oligonucleotide
gtgtgtgtcatgtgtggcag

SEQ ID NO: 33 - DNA, 20 bp, Artificial Sequence
  Description of Artificial Sequence Synthetic oligonucleotide
gcgcatgcgccgctggggcg

SEQ ID NO: 34 - DNA, 23 bp, Artificial Sequence
  Description of Artificial Sequence Synthetic oligonucleotide
gcagacgcgaggaaggagggcgc

SEQ ID NO: 35 - DNA, 19 bp, Artificial Sequence
  Description of Artificial Sequence Synthetic oligonucleotide
ggtagcaaagtgacgccga

SEQ ID NO: 36 - DNA, 20 bp, Artificial Sequence
  Description of Artificial Sequence Synthetic oligonucleotide
gctccagtagccaccgcatc

SEQ ID NO: 37 - DNA, 20 bp, Artificial Sequence
  Description of Artificial Sequence Synthetic oligonucleotide
gcctatatagtgcgggtggg

SEQ ID NO: 38 - DNA, 20 bp, Artificial Sequence
  Description of Artificial Sequence Synthetic oligonucleotide
gtgagtcgaggagaaacgac

SEQ ID NO: 39 - DNA, 36 bp, Artificial Sequence
  Description of Artificial Sequence Synthetic oligonucleotide
ttgttatccgctcacaattccacatgtggccacaaa

SEQ ID NO: 40 - DNA, 22 bp, Artificial Sequence
  Description of Artificial Sequence Synthetic oligonucleotide
cccacactctcaccataagagc

SEQ ID NO: 41 - DNA, 21 bp, Artificial Sequence
  Description of Artificial Sequence Synthetic oligonucleotide
ttgccttgtgccttgccttgc